SEQUENCE LISTING (15 sequences)

SEQ ID NO: 1 - DNA, 1428 bp, Artificial Sequence
  Description of Artificial Sequence Synthetic polynucleotide
ggtacctagctgcagccccgttgtcaggtgtttacgccagaaaaggtcctccagctgctttaacattaccacgtgttcaatgtcgtgcttcacgttatccaattgctgttgattgttcatggacattacctccagctccaaattcaacatcaccagtttcatttattgctacatatcgtcttggtatggctgctcgtggtcattcatggccatgtttacaacaaacaccaacatcaacatcatgtacaattacagatgttcaattattttcaatggctccatatgttttaaatgttacagctgttcatccctggggttcatcatcatcatttgttccatttattacagaacatattattaaaccagatccaccagaaggtgttcgtttatcaccattagctgaacgtcaacttcaagttcaatgggaaccaccaggttcatggccatttccagaaattttttcattaaaatattggattcgttataaaagacaaggtgctgctcgttttcatcgtgttggtccaattgaagctacatcatttattcttcgtgctgttcgtccacgtgctcgttattatgttcaagttgctgcacaagatttaactgattatggtgaattatcagattggtcacttccagctacagctacaatgtcattaggtaaatcacgtggttcaggttcaggtggatcaggtggttcaggatctggtaaattatttccacgtccaccaggtcgtccacaattatcattacaagaattacgtcgtgaatttacagtttcattacatttagctcgtaaacttttatctgaagttcgtggtcaagctcatcgttttgctgaatcacatcttccaggtgttaatctttatttattaccacttggtgaacaacttccagatgtttcattaacatttcaagcttggcgtcgtctttcagatccagaacgtctttgttttatttcaacaacattacaaccatttcatgctttattaggtggtcttggtacacaaggtcgttggacaaatatggaacgtatgcaattatgggctatgcgtttagatttacgtgatcttcaacgtcatttacgttttcaagttttagctgctggttttaatttacctgaagaagaagaagaagaagaagaagaagaagaagaagaacgtaaaggattacttccaggtgctttaggttcagctttacaaggtccagcacaagtttcatggccacaattactttcaacatatcgtttattacattcattagaattagttttatcaagagctgttcgtgaattattattattatcaaaagctggtcattcagtttggccattaggttttccaacattatcaccacaaccataaactagtcgcataaccccttggggcctctaaacgggtcttgaggggttttctgcagtctgagctc

SEQ ID NO: 2 - protein, 451 aa, Artificial Sequence
  Description of Artificial Sequence Synthetic polypeptide
AAAPLSGVYARKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKSRGSGSGGSGGSGSGKLFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 3 - DNA, 1407 bp, Artificial Sequence
  Description of Artificial Sequence Synthetic polynucleotide
ggcgcgcctagctgcagccccgttgtcaggtgtttacgccggttatacagaaacagctttagttgctttatcacaaccacgtgttcaatgtcatgcttcacgttatccagttgctgttgattgttcatggacaccattacaagctccaaattcaacacgttcaacatcatttattgctacatatcgtttaggtgttgctacacaacaacaatcacaaccatgtttacaacgttcaccacaagcttcacgttgtacaattccagatgttcatttattttcaacagttccatatatgttaaatgttacagctgttcatccaggtggtgcttcatcatcattattagcttttgttgctgaacgtattattaaaccagatccaccagaaggtgttcgtttacgtacagctggtcaacgtttacaagttttatggcatcctccagcttcatggccatttccagatattttttcattaaaatatcgtttacgttatcgtcgtcgtggtgcttcacattttcgtcaagttggtccaattgaagctacaacattcacattacgtaattcaaaaccacatgctaaatattgcattcaagtttcagctcaggatttaactgattatggtaaaccatcagattggtcattaccaggtcaagttgaatcagctccacataaaccatcacgtggttcaggttcaggtggatcaggtggttcaggatctggtaaattaccaactgatccattatcattacaagaattacgtcgtgaatttacagtttcattgtatttagctcgtaaattattatctgaagttcaaggatatgttcattcatttgctgaatcacgtttaccaggtgttaatttagatttacttccattaggatatcatttaccaaatgtttcattaacatttcaagcttggcatcatttatcagattcagaacgtttatgttttttagctacaactttacgtccatttccagctatgcttggtggtttaggtacacaaggtacatggacatcatcagaacgtgaacaattatgggctatgcgtttagatttacgtgatttacatcgtcatttacgttttcaagttttagctgctggttttaaatgttcaaaagaagaagaagataaagaggaggaagaagaagaggaagaggaagaaaaaaaattaccattaggtgctttaggtggtccaaatcaagtttcaagtcaagtttcatggccacaattattatatacatatcagcttttgcattctttagaacttgttttaagtcgtgctgttcgtgatttattattattatcattaccacgtcgtccaggttcagcttgggattcataaactagtcgcataaccccttggggcctctaaacgggtcttgaggggttttctgcagtcttaattaa

SEQ ID NO: 4 - protein, 443 aa, Artificial Sequence
  Description of Artificial Sequence Synthetic polypeptide
AAAPLSGVYAGYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPSRGSGSGGSGGSGSGKLPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 5 - protein, 17 aa, Artificial Sequence
  Description of Artificial Sequence Synthetic peptide
SRGSGSGGSGGSGSGKL

SEQ ID NO: 6 - DNA, 1371 bp, Mus sp.
ggcgcgcctagctgcagccccgttgtcaggtgtttacgccggatatacagaaacagctttagttgctttatcacaaccacgtgttcaatgtcatgcttcacgttatccagttgctgttgattgttcatggacaccattacaagctccaaattcaacacgttcaacatcatttattgctacatatcgtttaggtgttgctacacaacaacaatcacaaccatgtttacaacgttcaccacaagcttcacgttgtacaattccagatgttcatttattttcaacagttccatatatgttaaatgttacagctgttcatccaggtggtgcttcatcatcattattagcttttgttgctgaacgtattattaaaccagatccaccagaaggtgttcgtttacgtacagctggtcaacgtttacaagttttatggcatcctccagcttcatggccatttccagatattttttcattaaaatatcgtttacgttatcgtcgtcgtggtgcttcacattttcgtcaagttggtccaatcgaagctacaacatttacattacgtaattcaaaaccacatgctaaatattgtattcaagtttcagcacaggatttaactgattatggtaaaccatcagattggtcattaccaggtcaagttgaatcagctccacataaaccatcacgtggttcaggttcaggtggatcaggtggttcaggatctggtaaattacgtgttattccagtttcaggtccagctcgttgtttatcacaatcacgaaatttattaaaaacaactgatgatatggttaaaacagctcgtgaaaaattaaaacattattcatgtacagctgaagatattgatcatgaagatattacacgtgatcaaacatctacattaaaaacatgtttaccacttgaattacataaaaacgaacgttgtcttgctacacgtgaaacatcatcaacacgtcgtggttcatgtttaccaccacaaaaaacaagtttaatgatgacattatgtttaggttcaatttatgaagatcttaaaatgtatcaaacagaatttcaggcaattaatgctgctttacaaaatcataatcatcagcagattattcttgataaaggtatgcttgttgctattgatgaattaatgcaatcattaaatcataatggtgaaacattaagacaaaaaccaccagttggtgaagctgatccatatagagttaaaatgaaattatgcattcttttacatgctttttctacacgtgttgttacaattaatcgtgttatgggttatttatcatcagcttaaactagtcgcataaccccttggggcctctaaacgggtcttgaggggttttctgcagtcttaattaa

SEQ ID NO: 7 - protein, 431 aa, Mus sp.
AAAPLSGVYAGYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPSRGSGSGGSGGSGSGKLRVIPVSGPARCLSQSRNLLKTTDDMVKTAREKLKHYSCTAEDIDHEDITRDQTSTLKTCLPLELHKNERCLATRETSSTRRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 8 - DNA, 16 bp, Artificial Sequence
  Description of Artificial Sequence Synthetic oligonucleotide
cctagctgcagccccg

SEQ ID NO: 9 - DNA, 19 bp, Artificial Sequence
  Description of Artificial Sequence Synthetic oligonucleotide
agactgcagaaaacccctc

SEQ ID NO: 10 - protein, 27 aa, Lactococcus lactis
MKKKIISAILMSTVILSAAAPLSGVYA

SEQ ID NO: 11 - protein, 30 aa, Artificial Sequence
  Description of Artificial Sequence Synthetic polypeptide
MKKNIISAILMSTVILSAAAPLSGVYADTN

SEQ ID NO: 12 - DNA, 32 bp, Artificial Sequence
  Description of Artificial Sequence Synthetic oligonucleotide
tagctgcagccccgttgtcaggtgtttacgcc

SEQ ID NO: 13 - protein, 211 aa, Mus sp.
GYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 14 - protein, 205 aa, Mus sp.
PTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 15 - DNA, 61 bp, Artificial Sequence
  Description of Artificial Sequence Synthetic oligonucleotide
taaactagtcgcataaccccttggggcctctaaacgggtcttgaggggttttctgcagtct

(19) United States
(12) Patent Application Publication
(10) Pub. No.: US 2013/0164380 A1
(43) Pub. Date: Jun. 27, 2013

(54) COMPOSITIONS AND METHODS FOR TREATING INFLAMMATORY CONDITIONS

(71) Applicants: Scott K. Durum, Frederick, MD (US); Lothar Steidler, Lokeren (BE)

(72) Inventors: Scott K. Durum, Frederick, MD (US); Lothar Steidler, Lokeren (BE)

(73) Assignees: ActoGeniX NV, Zwijnaarde (BE); The United States of America, as represented by the Secretary, National Institute of Health, Rockville, MD (US)

(21) Appl. No.: 13/704,956

(22) PCT Filed: Jun. 17, 2011
(86) PCT No.: PCT/US2011/040952

§ 371 Date: Mar. 4, 2013

Related U.S. Application Data
(60) Provisional application No. 61/355,965, filed on Jun. 17, 2010.

(51) Int. Cl. A61K 9/00

(52) U.S. Cl. CPC A61K 9/0053
USPC 424/490; 424/85.2; 424/93.2; 435/252.3

(57) ABSTRACT

The present invention provides methods and compositions for treating inflammatory bowel disease, including ulcerative colitis and Crohn's Disease, and other related conditions, by locally administering to the intestinal mucosa of a subject having inflammatory bowel disease a therapeutically effective amount of IL-27 or a therapeutic variant or fragment thereof. The invention further provides a method to treat inflammatory bowel disease comprising administering to the subject a recombinant microorganism capable of producing a therapeutically effective amount of IL-27 or a variant or fragment thereof in situ in the intestinal mucosa.

### CROSS-REFERENCE TO RELATED APPLICATION

This application claims the benefit of U.S. Provisional Application No. 61/355,965, filed Jun. 17, 2010, the contents of which are hereby incorporated by reference in its entirety.

### GOVERNMENT SUPPORT

This work was supported by the Intramural Research Program of the National Institutes of Health (HHSN2632009000071). The Government has certain rights to this invention.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND OF THE INVENTION

1. Field of the Invention

The present invention relates to the treatment of inflammatory bowel disorder, including ulcerative colitis and Crohn's Disease, by the administration of anti-inflammatory cytokines to the intestinal mucosa by a suitable delivery means, e.g., via delivery by a recombinant microorganism engineered to produce the desired cytokines or a microparticle that locally delivers the cytokines into the gastrointestinal tract of a subject.

2. Background

Inflammatory bowel disease refers to a group of gastrointestinal disorders characterized by a chronic nonspecific inflammation of portions of the gastrointestinal tract that involve aberrant activation of innate and adaptive immune responses. Approximately 1.4 million individuals are affected by inflammatory bowel disease in the United States alone. Ulcerative colitis and Crohn's Disease are the most prominent examples of inflammatory bowel disease in humans. They are associated with many symptoms and complications, including growth retardation in children, rectal prolapse, blood in stools (e.g., melena and/or hematochezia), wasting, iron deficiency, and anemia, for example, iron deficiency anemia and anemia of chronic disease or of chronic inflammation. In addition, inflammatory bowel disease greatly predisposes to colon cancer in that twenty percent of ulcerative colitis patients will eventually develop colon cancer unless the colon is surgically removed.

In general, inflammatory bowel disease is thought to reflect a breakdown in intestinal homeostasis with the development of aberrant inflammatory responses to intestinal bacteria. One line of evidence supporting this hypothesis of perturbed host-microbial interactions is that treatment with antibiotics has a modest effect on improving disease activity in patients. Additional evidence comes from the demonstration that inflammatory bowel disease patients harbor T cells specifically reactive to enteric bacteria, whereas normal patients lack such T cells. Mouse models of inflammatory bowel disease have been extensively investigated and support the concept that the immune system, overreacting to enteric microflora, can cause collateral damage to the bowel.

Ulcerative colitis more specifically refers to a chronic, nonspecific, inflammatory, and ulcerative disease having manifestations primarily in the colonic mucosa. It is frequently characterized by bloody diarrhea, abdominal cramps, blood and mucus in the stool, malaise, fever, anemia, anorexia, weight loss, leukocytosis, hypoalbuminemia, and an elevated erythrocyte sedimentation rate (“ESR”). Complications can include hemorrhage, toxic colitis, toxic megacolon, occasional rectovaginal fistulas, and an increased risk for the development of colon cancer. This condition is also associated with noncolon complications, such as arthritis, ankylosing spondylitis, sacroileitis, posterior uveitis, erythema nodosum, pyoderma gangrenosum, and episcleritis. Treatment varies considerably with the severity and duration of the disease. For instance, fluid therapy to prevent dehydration and electrolyte imbalance is frequently indicated in a severe attack. Additionally, special dietary measures are sometimes useful. Medications include various corticosteroids, sulphasalazine and some of its derivatives, and possibly immunosuppressive drugs.

Crohn's Disease shares many features in common with ulcerative colitis. Crohn's Disease is distinguishable in that lesions tend to be sharply demarcated from adjacent normal bowel, in contrast to the lesions of ulcerative colitis, which are fairly diffuse. Crohn's Disease predominately afflicts the ileum (ileitis) and the ileum and colon (ileocolitis). In some cases, the colon alone is diseased (granulomatous colitis) and sometimes the entire small bowel is involved (jejunoileitis). In rare cases, the stomach, duodenum, or esophagus are involved. Lesions include a sarcoid-type epithelioid granuloma in roughly half of the clinical cases. Lesions of Crohn's Disease can be transmural including deep ulceration, edema, and fibrosis, which can lead to obstruction and fistula formation as well as abscess formation. This contrasts with ulcerative colitis, which usually yields much shallower lesions, although occasionally the complications of fibrosis, obstruction, fistula formation, and abscesses are seen in ulcerative colitis as well.

Treatment is similar for both diseases and includes steroids, sulphasalazine and its derivatives, and immunosuppressive drugs such as cyclosporin A, mercaptopurine and azathioprine. In more recently developed treatments, systemic blockade of the inflammatory cytokine tumor necrosis factor-α (TNF-α) has been shown to be highly effective in some Crohn's cases, however about two-thirds of patients fail to respond. Moreover, there is concern that sustained neutralization of TNF-α can result in enhanced susceptibility to infection at other sites, for example, reactivation of tuberculosis. Thus, there is a great need for more specific therapeutic approaches for treating inflammatory bowel disease, including ulcerative colitis and Crohn's Disease and other inflammatory conditions of the gastrointestinal tract and related regions.

A more recent and alternative strategy to down-regulate inflammation associated with the gastrointestinal tract, including IBD, is through anti-inflammatory cytokine therapy, i.e., the administration of cytokines having properties that tend to reduce inflammation. For example, interleukin-10 (IL-10) is a powerful, exclusively anti-inflammatory cytokine which displays profound downregulation of all aspects of immune activity. IL-10 has recently been clinically evaluated. However, systemic administration of recombinant IL-10 has been abandoned altogether because of undesirable side effects.

In view of the above-mentioned problems in the art pertaining to currently known therapeutic strategies for treating IBD and other related inflammatory conditions of the gastrointestinal tract, new treatment strategies that minimize or avoid such problems are highly desirable.

### SUMMARY OF THE INVENTION

The present invention overcomes the problems in the art by providing a previously unknown treatment strategy for treating inflammatory bowel disease (“IBD”) and other related inflammatory conditions, including ulcerative colitis and Crohn's Disease, that involves the administration of the cytokine interleukin-27 (“IL-27”) to the intestinal mucosa, e.g., via local delivery to the GI tract using microorganism-based delivery systems, in a manner that is safe and effective, or as a microparticle suitable for controlled delivery of the cytokine in the gastrointestinal tract of a subject.

Interleukin 27 (IL-27) is a pleiotropic cytokine, having both pro-inflammatory (see Cox et al., J. Exp. Med. 208:115-123 (2010)) and anti-inflammatory activity (see Schmidt et al., Inflamm. Bowel Dis. 11:16-23 (2005)). The role of IL-27 in IBD pathology remains unresolved, and at present, it is not clear from the art how IL-27 can be delivered to enhance its anti-inflammatory and/or avoid its pro-inflammatory properties. Furthermore, when looking for alternatives to IL-10 for treating IBD, IL-27 is an unlikely choice because the anti-inflammatory properties of IL-27 are associated with the induction of IL-10.

The inventors, however, have surprisingly discovered that IL-27 is a strikingly more effective anti-inflammatory molecule than IL-10 when delivered in situ. Accordingly, the present invention provides a previously unknown, as well as unexpected, effective treatment strategy for treating inflammatory bowel disease, which involves local administration of therapeutic IL-27 directly to the intestinal mucosa, e.g., using a microbial delivery system or a microparticle that provides controlled delivery of the active ingredient into the gastrointestinal tract. The invention also avoids the negative effects associated with systemic delivery of immunosuppressive cytokines (see Colombel, Expert Rev. Gastroenterol. Hepatol. 2:163-176 (2008); Sandborn, Dig Dis. 28:536-542 (2010); and Van Assche et al., Curr. Opin. Gastroenter. 25:323-328 (2009)), and surprisingly avoiding pro-inflammatory activity of IL-27.

In one aspect, the invention provides methods for treating inflammatory bowel disease, mucosal inflammatory pathology or intestinal inflammatory pathology in a subject in need thereof. In embodiments, the methods involve locally administering to the intestinal mucosa of the subject a therapeutically effective amount of IL-27 or a therapeutic variant or fragment thereof.

In embodiments, the IL-27 is administered using a gastrointestinal delivery system.

In embodiments, the inflammatory bowel disease is Crohn's Disease or ulcerative colitis.

In one aspect, the invention provides methods for treating a condition sensitive to IL-27 in a subject in need thereof. In embodiments, the methods involve administering to the subject a recombinant microorganism capable of producing a therapeutically effective amount of IL-27 or a therapeutic variant or fragment thereof. In embodiments, the recombinant microorganism produces the IL-27 in situ in the intestinal mucosa of the subject.

In embodiments, the condition is an inflammatory condition in a tissue of the gastrointestinal tract, including inflammation of the intestine, stomach, liver, pancreas or peritoneum. In embodiments, the condition is an inflammatory or noninflammatory condition outside of the gastrointestinal system, including type I diabetes, severe food allergies, or celiac disease. In embodiments, the condition is colon cancer or another cancer of a tissue of the gastrointestinal tract.

In one aspect, the invention provides microparticles containing IL-27. In embodiments, the microparticles are suitable for release of the active ingredient in the gastrointestinal tract. In related embodiments, the microparticles have a coating that enables controlled release of the IL-27 or therapeutic variant or fragment thereof into the gastrointestinal tract.

In one aspect, the invention provides pharmaceutical compositions having any microparticle described herein. In embodiments, the pharmaceutical compositions have the above-described microparticles.

In one aspect, the invention provides pharmaceutical compositions having a recombinant microorganism capable of producing a therapeutically effective amount of IL-27 in situ in a tissue of the gastrointestinal tract.

In a related aspect, the invention provides pharmaceutical compositions having Lactococcus lactis that is capable of expressing a therapeutically effective amount of IL-27 in situ in a tissue of the gastrointestinal tract.

In one aspect, the invention provides kits that contain a recombinant microorganism capable of producing a therapeutically effective amount of IL-27 in situ in the intestinal mucosa. In embodiments, the kit contains instructions for use in treating inflammatory bowel disease. In embodiments, the inflammatory bowel disease is Crohn's Disease or ulcerative colitis.

In one aspect, the invention provides kits that contain microparticles suitable for release of the active ingredient in the gastrointestinal tract. In embodiments, wherein the microparticle contains IL-27. In embodiments, the microparticle has a formulation or coating that enables controlled release of the IL-27 or therapeutic variant or fragment thereof into the gastrointestinal tract. In related embodiments, the coating further enables continuous or sustained release of the IL-27 or therapeutic variant or fragment thereof. In embodiments, the kit contains instructions for use in treating inflammatory bowel disease. In embodiments, the inflammatory bowel disease is Crohn's Disease or ulcerative colitis.

In any of the above aspects and embodiments, the therapeutically effective amount of the IL-27 is sufficient to reduce intestinal mucosal inflammation by at least 10-25%, 25-50%, 10-50%, 50-90%, 50-75%, 50-70%, 50-80%, 50-90%, 60-70%, 60-80%, 60-90%, 70-80%, 80-90%, 90-95%, 90-99%, or 95-99%. In embodiments, the therapeutically effective amount of the IL-27 is sufficient to reduce intestinal mucosal inflammation by at least 10-90%. In related embodiments, the therapeutically effective amount of the IL-27 is sufficient to reduce intestinal mucosal inflammation by at least 70-80%.

In any of the above aspects and embodiments, the methods involve co-administering a second therapeutic agent or the kits contain a second therapeutic agent. In embodiments, the second therapeutic agent is a corticosteroid, sulphasalazine, derivative of sulphasalazine, immunosuppressive drug, cyclosporin A, mercaptopurine, azathioprine, cytokine, or cytokine antagonist. In related embodiments, the cytokine or cytokine antagonist is tumor necrosis factor-α antagonist, IL-10, IL-27, or IL-35.

In embodiments, the co-administered second therapeutic agent is by intravenous, parenteral, oral or transdermal administration. In related embodiments, the co-administered second therapeutic agent is administered using a gastrointestinal delivery system.

In any of the above aspects and embodiments, the gastrointestinal delivery system is a recombinant microorganism effective to produce the IL-27 in situ in the intestinal mucosa in the subject.

In any of the above aspects and embodiments, the recombinant microorganism any microflora species described herein, including, but not limited to, microflora species belonging to i) the bacterial genera of Bacteriodes, Clostridium, Fusobacterium, Eubacterium, Ruminococcus, Peptococcus, Eschericia, Lactobacillus, Enterococcus, or Lactococcus; ii) the yeast genera of Hansenula, Kluiveromyces, Pichia, Saccharomyces, or Schizosaccharomyces, and iii) the fungi genera of Candida, Saccharomyces, Aspergillus or Penicillium. In embodiments, the gastrointestinal delivery system is a bacterial species that belows to the Lactoccus or Enterococcus genera. In related embodiments, the bacterial species is L. lactis or E. faecium. In some embodiments, the bacterial species is L. lactis faecium or any subspecies and strains thereof, such as, without limitation Lactococcus lactis ssp. cremoris, Lactococcus lactis ssp. hordniae, Lactococcus lactis ssp. lactis, Lactococcus lactis ssp. bv. diacetylactis. In some embodiments, the bacterial species is E. faecium or any subspecies and strains thereof, such as, without limitation E. faecium strain LMG15709.

In embodiments, the gastrointestinal delivery system is a non-commensal and non-colonizing bacterial species. In related embodiments, the bacteria is a gram positive bacteria. In some embodiments, bacterial species belongs to the Lactococcus or Enterococcus genera.

In embodiments, the gastrointestinal delivery system is a microparticle comprising IL-27 or therapeutic variant or fragment thereof. In related embodiments, the microparticle further comprises a formulation that enables controlled release of the IL-27 or therapeutic variant or fragment thereof into the gastrointestinal tract. In related embodiments, the microparticle further comprises a coating that enables controlled release of the IL-27 or therapeutic variant or fragment thereof into the gastrointestinal tract. The coating may be any coating described herein or well-known in the art that can provide controlled release of an agent. In embodiments, the coating further enables continuous or sustained release of the IL-27 or therapeutic variant or fragment thereof.

In any of the above aspects and embodiments, the subect can be a mammal. In embodiments, the subject is a human.

In any of the above aspects and embodiments, the IL-27 is encoded by the nucleotide sequence of SEQ ID NO: 1 (human) or SEQ ID NO: 3 (mouse).

In any of the above aspects and embodiments, the IL-27 has the amino acid sequence of SEQ ID NO: 2 (human) or SEQ ID NO: 4 (mouse).

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 provides a schematic illustrating IL-27 production and its activities.

FIGS. 2A-2C show that genetically engineered L. lactis can express IL-27 (LL-IL-27) and IL-35 (LL-IL-35). FIG. 2A includes a gel. Supernatants were collected from cultures of engineered L. lactis expressing either mouse IL-27 or mouse IL-35 and the proteins contained therein were separated by SDS-PAGE. Detectable anti-Ebi3 antibodies (“Ebi3” is the beta chain component of both IL-27 and IL-35) were used to detect IL-27 and IL-35 by Western blot. FIG. 2B includes a gel. Biological activity of IL-27 was measured by its ability to stimulate lymphocytes and induce phosphorylation of Stat 1 and Stat3 detected by Western blot. The results demonstrate that IL-27 produced by engineered L. lactis is active, resulting in the phosphorylation of Stat 1 and Stat3. FIG. 2C includes a graph. Biological activity of IL-27 was also measured by increased IL-10 secretion determined by ELISA and Tbet mRNA production as determined by quantitative PCR. rIL-27=commercial recombinant IL27; LL-IL-27=recombinant L. lactis expressing IL-27.

FIGS. 3A and 3B show that LL-IL-27 is delivered locally in vivo. LL-IL-27 was administered to normal C57Bl/6 male mice by oral gavage. FIG. 3A includes a graph showing the recovery of L. lactis from the bowels of treated mice. Twelve hours after LL-IL-27 administration, different regions of the bowel were analyzed for erythromycin resistant bacterial colonies. Significant numbers of colony-forming units (CFU) were detected throughout the gut (representative results from two mice are shown). Living L. lactis was recovered from stomach; duodenum; jejunum; ileum; cecum; and in the proximal, terminal and distal colon. FIG. 3B includes a graph showing IL-10 levels in treated mice. Six hours after gavage, IL-10 was detected in the luminal contents of various regions of LL-IL-27-treated mice (designated T) compared to LL-vector control-treated mice (designated C). The results demonstrate that L. lactis-IL-27 given by oral gavage is capable of acting locally in the target organ.

FIG. 4 includes a graph showing the therapeutic effect of LL-IL-27. The T cell transfer model of inflammatory bowel disease (IBD) was used to evaluate any potential therapeutic benefit of LL-IL-27. Treatment was begun as symptoms developed, e.g., six weeks after transfer of CD45RB(hi) T cells in Rag1^{-/-} hosts. At day 69, IBD mice treated with LL-IL-27 (n=5) were all healthy, while no mice treated with LL-control vector (n=5) survived to the end point.

FIGS. 5A-5J include histological stains showing that LL-IL-27 protects the distal colon from destruction of villi and inflammatory infiltration. No pathology was observed in the cells of the IL-27 group except for a slight cellular infiltrate in one IBD mouse, compared to severe pathology in the LL-vector control group or another group that received LL-IL-35. Sections of distal colon (2 cm) were fixed in formalin, embedded in paraffin, and H&E staining was carried out according to routine procedure. FIGS. 5A-5D: Untreated IBD mice. A) 4×, Colon mucosa greatly thickened with inflammatory infiltrate and hyperplastic crypts. B) 10×, Hyperplastic crypts depleted of goblet cells, crypt abscesses and inflammatory infiltrate in the mucosa. C) 10×, Gut intraepithelial neoplasia, crypt abscess and inflammatory infiltrate in the mucosa. D) 40×, Inflammatory infiltrate of mononuclear cells, neutrophils and eosinophils. FIGS. 5E-5G: LL-IL-27 treated mice. E) 4×, Colon mucosa has normal histology. F) 10×, Normal colon crypts with goblet cells. G) 40×, Normal colon crypts with goblet cells, no inflammatory infiltrate. FIGS. 5H-5J: LL-IL-35 treated IBD mice. H) 10×, Colon mucosa thickened with inflammatory infiltrate and hyperplastic crypts. I) 10×, Hyperplastic crypts depleted of goblet cells and inflammatory infiltrate in the mucosa. J) 40×, Inflammatory infiltrate of mononuclear cells, neutrophils and eosinophils.

FIG. 6 includes a graph showing the protection afforded by LL-IL-27 versus untreated mice (“UT”), LL-vector mice, and LL-IL-35 mice. Protection was measured by several parameters of inflammatory bowel disease and reflected in the Disease Activity Index (DAI) (see Ostanin et al., Am. J. Physiol. Gastrointest. Liver Physiol. 296:G135-G146 (2009), which is incorporated herein by reference for more details regarding the T cell transfer model of chronic colitis). LL-IL-27 protected completely from appearance of occult blood in stool. In addition, IBD mice treated with LL-IL-27 were associated with nearly normal stool consistency and partially relieved weight loss.

FIGS. 7A and 7B show that administration of LL-IL-27 reduces the transcript levels of inflammatory cytokines in treated IBD mice. FIG. 7A includes a graph showing the results of RT-PCR analysis on the transcript levels of inflammatory cytokines in distal colons from LL-IL-27 IBD mice, LL-vector IBD mice, and normal healthy mice. Significant reductions in transcripts were observed for TNFα, IL-6, IFNγ, IL-23, and IL-4 in the LL-IL-27 group relative to the vector group. Transcripts for IL-17A, IL-17F and RORγt, markers of Th17 cells, were also reduced. FIG. 7B includes a representative gel of the RT-PCR products.

FIG. 8 provides the nucleotide sequence of a recombinant human IL-27, wherein the alpha chain sequence and the EBI3 chain sequence have been fused. The sequence is identified as SEQ ID NO: 1.

FIG. 9 provides the amino acid sequence corresponding to SEQ ID NO: 1, which contains the alpha chain fused to the EBI3 chain by a linker having the sequence “SRGSGSGGSGGSGSGKL” (SEQ ID NO: 5). The amino acid sequence is identified as SEQ ID NO: 2.

FIG. 10 provides the nucleotide sequence of a recombinant mouse IL-27, wherein the alpha chain sequence and the EBI3 chain sequence have been fused. The sequence is identified as SEQ ID NO: 3.

FIG. 11A shows the amino acid sequence corresponding to SEQ ID NO: 3, which contains the alpha chain fused to the EBI3 chain by a linker having the sequence “SRGSGSGGSGGSGSGKL”. The sequence is identified as SEQ ID NO: 4. FIG. 11B provides a detailed schematic of the mouse IL-27 construct.

FIGS. 12A and 12B illustrate the construction of mouse and human IL-27. FIG. 12A provides a schematic of the construction of the DNA expression vector, pAGX0766, which encodes mouse IL-27 and can be used to transform L. lactis. FIG. 12B provides a schematic of the construction of the DNA expression vector, pLLhIL-27, which encodes human IL-27 and can be used to transform L. lactis.

FIGS. 13A and 13B show the sequence of mouse IL-35. FIG. 13A provides the nucleotide sequence (SEQ ID NO: 6) and FIG. 13B shows the corresponding amino acid sequence (SEQ ID NO: 7).

FIG. 14 includes a graph showing that induction of IL-10 requires the presence of T cells in Rag1^{-/-} mice. Rag^{-/-} lack T cells and the graph shows that there is no induction of IL-10 by LL-IL-27. IBD is induced in Rag^{-/-} mice following transfer of T cells, and the graph shows that LL-IL-27 induces IL-10 in these mice.

FIGS. 15A and 15B characterize the IL-10 producing T cells in IBD mice treated with LL-IL-27. Intraepithelial cells (IEL) were isolated from healthy C57Bl/6 mice, IBD Rag1^{-/-} untreated (UT) mice, IBD mice treated with LL-control vector, and IBD mice treated with LL-IL-27. FIG. 15A includes flow cytometry results showing the presence of a prominent CD4⁺CD8⁺ population in LL-IL-27 IBD mice. In contrast, healthy C57Bl/6 mice showed a predominance of CD8 cells; and IBD Rag1^{-/-} untreated mice and IBD mice treated with LL-vector showed CD4 infiltration. FIG. 15B includes flow cytometry results. IBD was induced using induced using IL-10 reporter T cells. The results show that the most prominent reporter expression observed in LL-IL-27 IBD mice was CD4⁺ CD8⁺ cells.

FIG. 16 demonstrates that IL-27 confers enhanced protection in vivo as compared to IL-10. The effects of recombinant L. lactis expressing IL-27 (LL-IL-27) or IL-10 (LL-IL-10) were evaluated in the mouse model of IBD. FIG. 16 includes a graph showing survival of LL-IL-27 and LL-IL-10 mice. The results indicate that although IL-10 delayed death, none of the LL-IL-10 mice survived. In contrast, LL-IL-27 conferred substantially more protection. More LL-IL27 mice survived, and those mice that died experienced a longer period of survival.

FIG. 17 shows expression of human interleukin-27 (hIL27) by Enterococcus faecium. FIG. 17 includes a graph depicts the quantification of human hIL27 secretion by E. faecium strains sAGX0270 (negative control) and sAGX0317. The amount of secreted hIL27 was expressed as ng/10⁹ CFU cells in 3 hours and indicated on the Y-axis. The results demonstrate that E. faecium strain sAGX0317 is able to efficiently secrete heterologous hIL27.

### DETAILED DESCRIPTION OF THE INVENTION

The present invention is based, at least in part, on the unexpected discovery that IL-27, a cytokine known to have both immunosuppressive and immunostimulative (or anti- and pro-inflammatory) characteristics (i.e., referred to as the pleiotropic nature of IL-27) provides a therapeutic benefit for the treatment of inflammatory bowel disease and other conditions sensitive to IL-27. The pleiotropic characteristics of IL-27 and the failure of IL-10 as a therapeutic molecule to treat IBD make IL-27 an unlikely candidate to treat IBD. Nevertheless, the present inventors have surprisingly discovered the profound effectiveness of IL-27 in treating IBD when using the methods and techniques of the present invention, e.g., the local delivery via microorganism-based delivery systems or microparticles having a coating that provides for controlled delivery in the gastrointestinal tract of a subject.

### Inflammatory Bowel Disease and Other Conditions Treatable by the Present Invention IBD

As further background, inflammatory bowel disease (may be referred to herein as “IBD”) is a collective term for ulcerative colitis (may be referred to herein as “UC”) and Crohn's Disease (may be referred to herein as “CD”), which are regarded as different disorders, but have many common features and probably share at least some pathologic mechanisms. Rarely, a definitive diagnosis of neither Crohn's Disease nor ulcerative colitis can be made because of idiosyncrasies in their presentations. Both may present with a variety of overlapping symptoms, including abdominal pain, vomiting, diarrhea, rectal bleeding, weight loss and various associated disorders, including, arthritis, liver and eye problems, skin manifestations, pyoderma gangrenosum and primary sclerosing cholangitis. Diagnosis is generally by colonoscopy with biopsy of pathological lesions.

Typically, CD can affect any part of the gastrointestinal tract, from mouth to rectum. By contrast, UC is mainly restricted to the colon and rectum. Further, UC is typically restricted to the mucosa epithelial lining of the gut, whereas CD can affect the entire bowel wall. Thus, UC mostly appears in the colon, proximal to the rectum, and the characteristic lesion is a superficial ulcer of the mucosa; CD can appear anywhere in the bowel, with occasional involvement of stomach, esophagus and duodenum, and the lesions are usually described as extensive linear fissures.

The exact etiology of these diseases is unknown and the initial lesion has not been clearly defined; however, patchy necrosis of the surface epithelium, focal accumulations of leukocytes adjacent to glandular crypts, and an increased number of intraepithelial lymphocytes and certain macrophage subsets have been described as putative early changes, especially in Crohn's disease. One theory is that IBD reflects a breakdown in intestinal homeostasis with development of aberrant inflammatory responses to intestinal bacteria. One line of evidence supporting this hypothesis of perturbed host-microbial interactions is that treatment with antibiotics has a modest effect on improving disease activity in patients. Additional evidence comes from the demonstration that IBD patients harbor T cells specifically reactive to enteric bacteria, whereas normal patients lack such T cells. Also, mouse models of IBD have been extensively investigated and support the concept that the immune system, overreacting to enteric flora, can cause collateral damage to the bowel. Nevertheless, a combination of factors, including abnormalities in the immune system, genetic predisposition, environmental and psychological factors, may be of importance in determining the outcome of the disease.

Depending on the level of severity, IBD may require immunosuppression to control the symptom, such as prednisone, TNF inhibition, azathioprine (Imuran), methotrexate, or 6-mercaptopurine. More commonly, treatment of IBD requires a form of mesalamine. Often, steroids are used to control disease flares and were once acceptable as a maintenance drug. In use for several years in Crohn's disease patients and recently in patients with ulcerative colitis, biologicals have been used such as TNF inhibitors. Severe cases may require surgery, such as bowel resection, strictureplasty or a temporary or permanent colostomy or ileostomy. Alternative medicine treatments for bowel disease exist in various forms, however such methods concentrate on controlling underlying pathology in order to avoid prolonged steroidal exposure or surgical excisement.

Usually the treatment is started by administering drugs with high anti-inflammatory effects, such as prednisone. Once the inflammation is successfully controlled, the patient is usually switched to a lighter drug to keep the disease in remission, such as Asacol, a mesalamine. If unsuccessful, a combination of the aforementioned immunosuppression drugs with a mesalamine (which may also have an anti-inflammatory effect) may or may not be administered, depending on the patient.

Histoplasma produces toxins that cause intestinal disease called histoplasmosis that is a “serious consideration” in an immunocompromised patient with signs and symptoms of IBD. Antifungal drugs such as nystatin (a broad spectrum gut antifungal) and either itraconazole (Sporanox) or fluconazole (Diflucan) have been suggested as a treatment for IBD disorders such as Crohn's disease and ulcerative colitis that all share the same symptoms such as diarrhea, weight loss, fever, and abdominal pain. In particular, blockade of the inflammatory cytokine tumor necrosis factor-α (TNF-α) has been shown to be highly effective in some Crohn's cases, however about two thirds of patients fail to respond. Moreover, there is concern that sustained neutralization of TNF-α can result in enhanced susceptibility to infection at other sites, for example reactivation of tuberculosis. Thus there is a great need for more specific therapeutic approaches and the study of cytokines in IBD is hoped to reveal additional therapeutic targets. In view of the apparent shortcomings of the present treatments, there is a great medical need for new treatment options for inflammatory bowel disease, based upon a better understanding of the underlying immunological reasons for the disease, including the role of various cytokines in the disease pathogenesis. The present invention advantageously advances the art by providing new methods, compositions and kits for administering IL-27, or therapeutic variants or fragment thereof to subjects having inflammatory bowel disease, including Crohn's Disease and ulcerative colitis, for treating or reducing one or more symptoms of IBD. The pleiotropic characteristics of IL-27 (i.e., having both pro- and anti-inflammatory properties) make it an unlikely candidate to treat IBD; nevertheless the present inventors have discovered its useful in such treatment using the methods and techniques of the present invention, e.g., the local delivery methods via microorganism-based delivery systems. In one embodiment, the administration of the IL-27 is carried out using a gastrointestinal delivery system, which can include, for example, a recombinant bacterial strain, e.g., L. lactis or E. faecium, engineered to express and release IL-27 locally within the gastrointestinal tract following ingestion or oral delivery of the strain to the subject, thereby avoiding unwanted side-effects of alternative systemic delivery routes.

### Other Treatable Conditions

The present invention also provides a method for treating a condition which is sensitive to the administration of IL-27 by locally administering to the affected bodily site of the subject a recombinant microorganism capable of producing a therapeutically effective amount of IL-27 or a therapeutic variant or fragment thereof.

In certain embodiments, the condition can be an inflammatory condition of the intestine (e.g., including celiac disease, diverticulitis and appendicitis), stomach, liver, pancreas or peritoneum or other tissue of the gastrointestinal tract or digestive system. Such other conditions can include inflammatory conditions of the oral cavity, esophagus, pancreas, pancreatic duct, liver, gallbladder, duodenum, bile duct, small intestine (ileum), large intestine (colon), cecum, appendix, or rectum. Specific conditions affecting the gastrointestinal system that may be treatable by the methods, compositions and kits of the present invention can include, for example, diverticulitis (a common digestive disease particularly found in the large intestine which develops from diverticulosis and involves the formation of inflammed pouches (diverticula) on the outside of the colon), celiac disease (an autoimmune disorder of the small intestine that occurs in genetically predisposed people of all ages from middle infancy onward caused by an autoimmune reaction that develops against gluten protein), appendicitis (condition characterized by inflammation of the appendix), gastroenteritis (inflammation of the gastrointestinal tract, involving both the stomach and the small intestine and resulting in acute diarrhea and which is caused most often by an infection from certain viruses or less often by bacteria, their toxins, parasites, or an adverse reaction to something in the diet or medication), pancreatitis (chronic or acute inflammation of the pancreas due to various causes), or peptic ulcer disease.

In other embodiments, the condition can be outside the gastrointestinal system and can include, for example, type I diabetes, severe food allergies, and celiac disease.

In still other embodiments, the condition can be a cancer of the gastrointestinal tract or digestive system, which can be, for example, colon cancer, or a cancer of any tissue of the gastrointestinal tract or digestive system, e.g., a cancer occurring in the oral cavity, esophagus, pancreas, pancreatic duct, liver, gallbladder, duodenum, bile duct, small intestine (ileum), large intestine (colon), cecum, appendix, or rectum. As pointed out already, IBD is associated with at least a 20% increase in risk of developing colon cancer; thus, in a particular embodiment, the method of the invention can be used to treat colon cancer in which other anti-inflammatory drugs have been shown to slow cancer progression.

### Use of Terms

The present invention may be understood more readily by reference to the following detailed description of preferred embodiments of the invention and the Examples included therein. Before the present methods and techniques are disclosed and described, it is to be understood that this invention is not limited to specific analytical or synthetic methods as such may, of course, vary. It is also to be understood that the terminology used herein is for the purpose of describing particular embodiments only and is not intended to be limiting. Unless defined otherwise, all technical and scientific terms used herein have the meaning commonly understood by one of ordinary skill in the art to which this invention belongs.

As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein, the terms “biological sample” or “patient sample” or “test sample” or “sample” as used herein, refer to a sample obtained from an organism or from components (e.g., cells) of a subject or patient for the purpose of diagnosis, prognosis, or evaluation of a subject of interest. The sample can be, for example, gastrointestinal tissue (e.g., intestinal mucosa) containing an IBD-related lesion. In embodiments, such a sample may be obtained for the purpose of determining the outcome of an ongoing condition or the effect of a treatment regimen on a condition. The sample may be any biological tissue or fluid, including those pertaining to the diagnosis or analysis of IBD. The sample may be a clinical sample which is a sample derived from a patient having IBD. Such samples include, but are not limited to, any tissue or fluid of the gastrointestinal tract, including such tissue or fluids from the esophagus, stomach, duodenum (connection point between stomach and small intestine), small intestine, large intestine (colon), ileum (connection point between the small and large intestine), sigmoid colon, rectum, anus, feces or any tissue or fluid obtained from any area of the body affected by the underlying IBD, including the eye, liver, kidney, skin, connective tissue (for associated arthritis attributed to IBD), or any other organ or tissue or fluid affected by the underlying IBD.

The term “subject” or “patient” refers to an animal which is the object of treatment, observation, or experiment. By way of example only, a subject includes, but is not limited to, a mammal, including, but not limited to, a human or a non-human mammal, such as domestic animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and the like.

As used herein, the term “specifically binds to” or is “specific for” a particular receptor or binding partner, e.g., in the context of a microsphere delivery system for delivering the IL-27 of the invention to a tissue of the gastrointestinal tract, is one that binds to that particular receptor or binding partner without substantially binding to any other receptor or molecule.

As used herein, the term “treatment” or “treating” includes any process, action, application, therapy, or the like, wherein a subject (or patient), including a human being, is provided with or administered an agent or composition (or recombinant organism expressing the agent of the invention) with the aim of improving the subject's condition, directly or indirectly, or slowing the progression of a condition or disorder in the subject (e.g., IBD, including Crohn's Disease or ulcerative colitis), or ameliorating at least one symptom of the disease or disorder under treatment (e.g., IBD, including Crohn's Disease or ulcerative colitis).

The term “combination therapy” or “co-therapy” means the administration of two or more therapeutic agents to treat a disease, condition, and/or disorder, e.g., IBD, including Crohn's Disease or ulcerative colitis. Such administration encompasses “co-administration” of two or more therapeutic agents in a substantially simultaneous manner. One therapy can be based on the embodiments of the invention pertaining to the administration of a recombinant microorganism engineered to express or produce IL-27 or a therapeutic fragment or variant thereof. A second therapy can be based on a known therapy for a disorder of the invention, e.g., IBD, including Crohn's Disease and ulcerative colitis, such as a therapeutic cytokine, e.g., tumor necrosis factor-α (TNF-α). The order of administration of two or more sequentially co-administered therapeutic agents is not limited. The administration of the two or more therapeutic agents may also be administered by different routes, e.g., by a local route (gastrointestinal delivery of agent using recombinant microorganism of the invention) and a systemic route (e.g., parenteral, injection, transdermal).

The phrase “therapeutically effective amount” means the amount of each agent of the invention (e.g., IL-27) administered by any route that will achieve the goal of improvement in a disease, condition, and/or disorder severity, and/or symptom thereof, while avoiding or minimizing adverse side effects associated with the given therapeutic treatment. In certain embodiments, the therapeutically effective amount is in relation to the delivery of a recombinant microorganism delivered to the gastrointestinal tract which said organism is engineered to express and release the therapeutically effective amount of the agent, e.g., the IL-27 of the invention. The determination of the therapeutically effective amount is within the skill set of those having ordinary skill in the art and may be determined with routine testing that would be done by such persons. It will be appreciated that such determination will be in part dependent upon by which administration route is utilized.

As used herein, the term “pharmaceutically acceptable” means that the subject item is appropriate for use in a pharmaceutical product.

As used herein, the term “therapeutic fragment or variant thereof” refers to the following. The term “therapeutic fragment” or, alternatively, “bioactive,” “biologically active,” or “biologically-active portion thereof,” refers to a fragment of IL-27 of the invention (or other polypeptide agents, e.g., the second therapeutic agents contemplated by the invention) that retains a substantial level of the biological activity of the full-size or native IL-27 of the invention, but preferably at least 99%, 95%, 90%, 85%, 80% 75%, 70%, 65% or 60% of its activity. The therapeutic fragment can be generated by any suitable means, as further discussed herein, by deletion of any terminal (N-terminal or C-terminal) or interior portion of the polypeptide such that it retains the above indicated level of activity. The term “therapeutic variant” refers to any IL-27 (or any other polypeptide agent of the invention) that may be made or obtained having any known chemical or biochemical protein or posttranslational modification produced biologically (i.e., in a cell) or chemically, e.g., glycosylation, acetylation, phosphorylation or the addition of lipids or carbohydrates, which result in a modified polypeptide that has at least (but may have increased activity) the above indicated level of activity. The variant can also refer to suitable agonists of IL-27, which may bind to and activate the IL-27 receptors with substantially the same effect, i.e., where the effect by the agonist is at least 99%, 95%, 90%, 85%, 80% 75%, 70%, 65% or 60% of the effect by native IL-27.

As used herein, the terms “amino acid” and “amino acids” refer to all naturally occurring L-α-amino acids. The amino acids are identified by either the single-letter or three-letter designations: Asp D aspartic acid; Ile I isoleucine; Thr T threonine; Leu L leucine; Ser S serine; Tyr Y tyrosine; Glu E glutamic acid; Phe F phenylalanine; Pro P proline; H is H histidine; Gly G glycine; Lys K lysine; Ala A alanine; Arg R arginine; Cys C cysteine; Trp W tryptophan; Val V valine; Gln Q glutamine; Met M methionine; and Asn N asparagine.

As will be understood, these amino acids may be classified according to the chemical composition and properties of their side chains. They are broadly classified into two groups, charged and uncharged. Each of these groups is divided into subgroups to classify the amino acids more accurately:

### I. Charged Amino Acids

Acidic Residues: aspartic acid, glutamic acid
Basic Residues: lysine, arginine, histidine

### II. Uncharged Amino Acids

Hydrophilic Residues: serine, threonine, asparagine, glutamine
Aliphatic Residues: glycine, alanine, valine, leucine, isoleucine
Non-polar Residues: cysteine, methionine, proline
Aromatic Residues: phenylalanine, tyrosine, tryptophan

As used herein, the term “homology” is defined as the percentage of residues in the candidate amino acid sequence that are identical with the residues in the amino acid sequence of their native counterparts (e.g., native IL-27) after aligning the sequences and introducing gaps, if necessary, to achieve the maximum percent homology by known methods (e.g., BLAST alignment tools). Methods and computer programs for the alignment are well-known in the art.

As used herein, “substitutional mutants” are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule. “Insertional mutants” are those with one or more amino acids inserted immediately adjacent to an amino acid at a particular position in a native sequence. Immediately adjacent to an amino acid means connected to either the carboxy or amino functional group of the amino acid. “Deletional mutants” are those with one or more amino acids in the native amino acid sequence removed. Ordinarily, deletional mutants will have one or two amino acids deleted in a particular region of the molecule.

As used herein, the term “symptoms” refers to any subjective evidence of disease or of a patient's condition. This includes evidence as perceived by the patient. Examples of symptoms of IBD include diarrhea, abdominal pain, fever, melena, hematochezia, and weight loss, and others as indicated herein elsewhere.

As used herein, the term “signs” refers generally to any objective evidence of a disease or condition, usually as perceived by an examining physician or features which would reveal themselves on a laboratory evaluation or other tests such as an ultrasonic study or a radiographic test. Some examples of signs of IBD include abdominal mass, glossitis, aphtous ulcer, anal fissure, perianal fistula, anemia, malabsorption, and iron deficiency. Occasionally, signs and symptoms overlap. For example, the patient complains of blood stools (a symptom), and a laboratory test of a stool sample is positive for blood (a sign).

As used herein, the term “cytokine” is meant a polypeptide factor produced transiently by a range of cell types, acting usually locally, and activating the expression of specific genes by binding to cell surface receptors.

As used herein, the term “IL-27” or “interleukin-27” refers to a two-polypeptide chain (comprising 1 alpha chain and 1 beta (or “Ebi3”) chain associated by non-covalent interactions in the native IL-27) cytokine that is utilized by the methods, composition, kits and other aspects of the present invention. The IL-27 can include native IL-27 expressed or encoded by human, mouse or any other mammal or animal. The IL-27 can also include any mutant version of IL-27 naturally produced by such animals (including humans) or those mutant versions constructed experimentally. The mutations can arise by any means, including deletions, insertions, substitutions, inversions, and the like. The mutant versions of IL-27 are meant to have at least 99%, 95%, 90%, 85%, 80% 75%, 70%, 65% or 60% of the activity of the native IL-27 from human, but may also, due to the particular mutation, may have increased activity as compared to the human sequence. In a particular embodiment, the IL-27 is recombinant in that it is a fusion of both the alpha chain and beta chain through a short polypeptide “linker”. Such a form may be referred to as an “IL-27 hyperkine,” as is referred to in the Examples. In embodiments, the IL-27 corresponds to a fusion of the human alpha and beta chains via the polypeptide linker having the sequence N—SRGSGSGGSGGSGSGKL-C (SEQ ID NO: 5), and having the amino acid sequence given by SEQ ID NO: 2 (and encoded by the nucleotide sequence given by SEQ ID NO: 1). In another embodiment, the IL-27 corresponds to a fusion of the mouse alpha and beta chains via the polypeptide linker having the sequence N—SRGSGSGGSGGSGSGKL-C (SEQ ID NO: 5), and having the amino acid sequence given by SEQ ID NO: 4 (and encoded by the nucleotide sequence given by SEQ ID NO: 2). However, the invention contemplates any suitable linker (described further herein elsewhere) to join the alpha and beta chains of IL-27, and in either order, according to the general formula of Formula I:

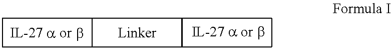

As used herein, the term “inflammatory bowel disease” or “IBD” refers to a group of inflammatory conditions of the colon, small intestine and other areas of the gastrointestinal tract, and which is described in more detail herein elsewhere. The major types of IBD are Crohn's Disease (“CD”) and ulcerative colitis (“UC”). IBD is inclusive as to the inflammatory conditions of the tissues of the gastrointestinal tract, as well as to the manifestations of the disease that occur in organ systems outside of the GI tract, including the skin, liver and other areas.

As used herein, the term “gram-positive bacterium” or “gram-positive bacteria” has its common meaning known in the art. By means of further guidance, a gram-positive bacterium/bacteria can be identified by Gram staining as retaining crystal violet stain.

As used herein, the term “isolated” or “purified” polypeptide or protein or biologically-active portion thereof is substantially free of cellular material or other contaminating proteins from the cell or tissue source from which the polypeptide, e.g., IL-27, is obtained.

The terms “nucleic acid molecule encoding”, “DNA sequence encoding”, and “DNA encoding” refer to the order or sequence of deoxyribonucleotides along a strand of deoxyribonucleic acid. The order of these deoxyribonucleotides determines the order of amino acids along the corresponding polypeptide chain encoded by that nucleic acid molecule. The DNA sequence thus codes for the amino acid sequence.

Nucleic acid is “operably linked” when it is placed into a functional relationship with another nucleic acid sequence. For example, DNA for a presequence or secretory leader (e.g., secretroy leader sequence for release or secretion of expressed protein from L. lactis or another recombinant microorganism of the invention) is operably linked to a DNA encoding a polypeptide (e.g., IL-27) if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, then synthetic oligonucleotide adaptors or linkers are used in accord with conventional practice.

The terms “replicable expression vector” and “expression vector” refer to a piece of DNA, usually double-stranded, which may have inserted into it a piece of foreign DNA. Foreign DNA is defined as heterologous DNA (e.g., DNA encoding IL-27), which is DNA not naturally found in the host cell (e.g., L. lactis or E. faecium). The vector is used to transport the foreign or heterologous DNA into a suitable host cell (e.g., L. lactis or E. faecium). Once in the host cell, the vector can replicate independently of the host chromosomal DNA, and several copies of the vector and its inserted (foreign) DNA may be generated. In addition, the vector contains the necessary elements that permit translating the foreign DNA into a polypeptide. Many molecules of the polypeptide encoded by the foreign DNA can thus be rapidly synthesized. The present invention also contemplates known methods for genetic manipulation of the genome of a host cell, e.g., L. lactis, with foreign DNA encoding a polypeptide of the invention such that the DNA encoding the desired polypeptide (e.g., IL-27) is integrated onto the host cell's own chromosome, which may provide greater stability of the foreign gene (IL-27).

As used herein, the term “transformation” means introducing DNA into an organism so that the DNA is replicable, either as an extrachromosomal element or by chromosomal integration.

As used herein, the term “transfection” refers to the taking up of an expression vector by a host cell whether or not any coding sequences are in fact expressed.

As used herein, the terms “transformed host cell” and “transformed” refer to the introduction of DNA into a cell. The cell is termed a “host cell”, and it may be a prokaryotic or a eukaryotic cell. Typical prokaryotic host cells include various strains of E. coli. In the present invention, the host cell can include in certain embodiments any normal microflora of the gastrointestinal tract, including Bacteriodes, Clostridium, Fusobacterium, Eubacterium, Ruminococcus, Peptococcus, Eschericia, Lactobacillus, or fungi, such as, Candida, Saccharomyces, Aspergillus or Penicillium. The host cells, in certain other embodiments, can also include non-commensal bacteria, which do not tend to colonize the gastrointestinal tract, such as, Lactococcus lactis or related organisms. Typical eukaryotic host cells are mammalian, such as Chinese hamster ovary cells or human embryonic kidney 293 cells. The introduced DNA is usually in the form of a vector containing an inserted piece of DNA. The introduced DNA sequence may be from the same species as the host cell or a different species from the host cell, or it may be a hybrid DNA sequence, containing some foreign and some homologous DNA.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. Compositions comprising such carriers are formulated by well-known conventional methods (see, e.g., Remington's Pharmaceutical Sciences, 14th Ed., Mack Publishing Col, Easton Pa. 18042, USA). Pharmaceutically acceptable excipients are well-known in the art have been amply described in a variety of publications, including, for example, A. Gennaro (2000) Remington: The Science and Practice of Pharmacy, 20th edition, Lippincott, Williams, & Wilkins; Remington's Pharmaceutical Sciences, 14th Ed. or latest edition, Mack Publishing Col, Easton Pa. 18042, USA; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

### IL-27 and Biologically Active Fragments or Variants Thereof.

The present invention is based, at least in part, on the unexpected discovery that IL-27, a cytokine known to have both immunosuppressive (anti-inflammatory) and immunostimulative (pro-inflammatory) characteristics, provides a therapeutic benefit for the treatment of inflammatory bowel disease.

In humans and mouse, IL-27 is a heterodimeric cytokine belonging to the IL-12 family of cytokines that is composed of two subunits, Epstein-Barr virus-induced gene 3 (Ebi3) (also known as IL-27B or IL-27 beta or the “beta” subunit) and IL27-p28 (also known as IL-30 or the “alpha” subunit). IL-27 are normally produced by antigen-presenting cells and plays a function in regulating the activity of B and T lymphocytes. See, for example, Larousserie et al., Am. J. Pathol. 166: 1217-1228 (2005) and U.S. Pat. No. 7,148,330, each of which is incorporated herein by reference in their entireties.

It is believed that IL-27's anti-inflammatory properties are based on its ability to induce expression of IL-10, which is a known and potent anti-inflammatory cytokine. As IL-10 is unquestionably an anti-inflammatory molecule, and because direct administration of IL-10 would yield more predictable results, IL-10 is the preferred choice for developing a therapeutic molecule for treating IBD. However, as described in Steidler et al., Ann N Y Acad Sci 1182:135-145 (2009), systemic administration of IL-10 resulted in severe adverse effects during clinical trials. As such, efforts in developing a systemically administered IL-10 therapeutic for treating IBD have been abandoned. The results described herein demonstrate that local delivery of IL-27 is highly effective at treating IBD. This finding is surprising because the profound effectiveness of IL-27 was neither predictable from previous findings in the art, nor was it specifically taught or suggested elsewhere in the art.

Indeed, the application of IL-27 as a therapeutic in IBD is not predictable based on the published literature, which contains reports that could be viewed as supportive of either beneficial or deleterious effects. For example, inhibition of IL-27 has been proposed as a treatment strategy for intestinal inflammation conditions, e.g., see WO 2008/071751 A1. The etiology of inflammatory disorders such as IBD is extremely complex, making it difficult to predict the effects of this candidate drug in vivo.

In addition, a number of observations might have predicted a deleterious outcome of L. lactis-IL-27 in IBD. This includes the initial descriptions of IL-27, characterizing it as promoting immune responses, not inhibiting immune responses as would be expected for an effective therapeutic in IBD (Pflanz et al, Immunity 16:779-90 (2002)). Other observations that might have predicted a negative outcome are that, in IBD, one chain of IL-27, EBI3, is constitutively expressed in intestine and the other chain, P28, is increased in inflammation of the intestine (Schmidt et al., Inflamm. Bowel Dis. 11:16-23 (2005); Masser et al., Immunology 112:437-445 (2004)). These observations suggest that overproduction of IL-27 might actually cause IBD, and would therefore have predicted an undesirable outcome for IL-27 as a therapeutic. In one mouse model of colitis, deletion of one of the IL-27 chains had no effect, neither exacerbating nor ameliorating IBD (Nieuwenhuis et al., PNAS, 99:16951-56 (2002)). A later paper in which part of the IL-27 receptor was deleted described worsened IBD (Honda et al., Inflamm. Bowel Dis. 11:1044-1052 (2005)), again predicting the opposite of a beneficial effect of IL-27 as a therapeutic in IBD.

Based on the published literature, IL-35 would have been a better candidate to have therapeutic benefit in IBD, because all reports consistently observed immunosuppressive effects of IL-35 (Bettinni et al., Current Opinion in Immunology 21:612-618 (2009)). However, as shown in the Examples of this application, L. lactis expressing IL-35 showed no therapeutic benefit in IBD in mice.

In one embodiment, the IL-27 of the invention is a chimeric polypeptide comprising both of the alpha and beta subunits covalently connected to one another by a linker, and has the following formula:

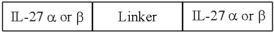

This format of the IL-27 has the advantage that, where the linker is an intervening polypeptide sequence, the IL-27 can be synthesized as a single chimeric polypeptide in bacterial hosts, such as, L. lactis or E. faecium. In addition, the IL-27 sequences of the invention can also be modified genetically such that the codon usage of the sequences is optimized for the recombinant microorganism in which expression of the gene will occur, e.g., optimized for L. lactis or E. faecium codon usage.

Thus, in one embodiment, the compositions, methods and kits of the invention utilize an IL-27 given by the amino acid sequence of SEQ ID NO: 2, which is encoded by the nucleotide sequence of SEQ ID NO: 1 and comprises the human alpha and beta chains of native human IL-27 joined by a polypeptide linker having the sequence N-SRGSGSGGSGGSGSGKL-C (SEQ ID NO: 5) and which has been optimized for codon usage for the host cell L. lactis and E. faecium.

In another embodiment, the compositions, methods and kits of the invention utilize an IL-27 given by the amino acid sequence of SEQ ID NO: 4, which is encoded by the nucleotide sequence of SEQ ID NO: 3 and comprises the human alpha and beta chains of native mouse IL-27 joined by a polypeptide linker having the sequence N-SRGSGSGGSGGSGSGKL-C (SEQ ID NO: 5) and which has been optimized for codon usage for the host cell L. lactis and E. faecium.

The linker sequence can be any suitable amino acid sequence, so long as it functions to allow the translation of the IL-27 as a single polypeptide and does not detract from the overall function of the IL-27. Preferably, the chimeric IL-27 should have about 100% the biological activity of the native IL-27, or the chimeric IL-27 can have at least about 99%, or at least 90% or at least about 80%, or at least about 70%, or at least about 60%, or at least about 50% of the biological activity of the native IL-27 cytokine.

In other embodiments, the IL-27 of the invention can include a leader sequence suitable to allow the polypeptide to be secreted or released from the bacterial delivery system (e.g., the L. lactis system). In certain embodiments, to enable secretion of IL-27, a fragment encoding a secretion leader suitable for use in L. lactis or E. faecium can be added to the 5′ end or 3′ end of the IL-27 sequence of the individual alpha and/or beta chain or of the sequence in which alpha and beta chains are linked. This will result in an IL-27 alpha-beta fusion protein or IL-27 alpha and IL-27 beta fusion proteins with N-terminal or C-terminal secretion leader extensions. This fragment can encode the well-known L. lactis secretion leader from the usp45 gene (van Asseldonk et al., Gene 95:155-160 (1990), which is incorporated herein by reference), having the sequence N-MKKKIISAILMSTVILSAAAPLSGVYA-C, but also any other seretion leader sequence which is functional in the microorganism vehicle used in the invention, e.g., L. lactis or E. faecium.

In embodiments that contemplate administration by means other than a recombinant microorganism, the present invention contemplates any form of IL-27, including the native form (e.g., isolated from human or mouse or other source), or forms whereby the alpha and beta subunits are covalently attached by a linker, chemically fused, or otherwise are joined in some suitable manner. Here, the linker could be a polypeptide linker or another type of linker which would suitably not diminish the biological activity of the IL-27. Preferable, the IL-27 used should have about 100% the biological activity of the native IL-27, or the IL-27 can have at least about 99%, or at least 90% or at least about 80%, or at least about 70%, or at least about 60%, or at least about 50% of the biological activity of the native IL-27 cytokine.

As mentioned previously, the present invention contemplates the use and administration of biologically active fragments and variants of IL-27. The construction of various biologically active fragments and variants of IL-27 is the subject of U.S. Pat. No. 7,148,330, which is hereby incorporated by reference in its entirety.

Amino acid sequence, glycosylation variants and covalent derivatives (e.g., chimeric variants, which can include fused alpha and beta chains of IL-27 from the same or different sources) of any native or recombinant IL-27 species and other biologically active fragments and/or variants of IL-27 can be prepared by methods known in the art. In one approach, particular regions or sites of the DNA encoding IL-27 can be targeted for mutagenesis, i.e., site-directed mutagenesis of IL-27. The mutations can be made using DNA modifying enzymes such as restriction endonucleases (which cleave DNA at particular locations) nucleases (which degrade DNA) and/or polymerases (which synthesize DNA). Restriction endonuclease digestion of DNA followed by ligation may be used to generate deletions, e.g., as described in section 15.3 of Sambrook et al., Molecular Cloning: A Laboratory Manual, second edition, Cold Spring Harbor Laboratory Press. New York, 1989.

Oligonucleotide-directed mutagenesis can also be used as a method for preparing substitution variants of IL-27. It may also be used to conveniently prepare the deletion and insertion variants that can be used in accordance with this invention. This technique is well-known in the art as described by Adelman et al., DNA 2:183 (1983)), among other known sources. The oligonucleotides can be readily synthesized using techniques well-known in the art, such as that described by Crea et al., Proc. Natl. Acad. Sci. USA 75:5765 (1978)). The production of single-stranded templates for use in this technique is described in sections 4.21-4.41 of Sambrook et al., supra.

PCR mutagenesis is also suitable for making the IL-27 variants that can be used in the methods of the present invention. The PCR technique is, for example, disclosed in U.S. Pat. No. 4,683,195; in section 14 of Sambrook et al., or in Chapter 15 of Current Protocols in Molecular Biology, Ausubel et al. eds., Greene Publishing Associates and Wiley-Interscience 1991.

The DNA encoding the IL-27 variants hereof can be inserted into a replicable expression vector for further cloning and expression. Many vectors are available, and selection of the appropriate vector will depend on 1) whether it is to be used for DNA amplification (cloning) or for expression, 2) the size of the DNA to be inserted into the vector, and 3) the host cell to be transformed with the vector. Each vector contains various components depending on its function and the host cell with which it is compatible. The vector components generally include, but are not limited to, one or more of the following: a signal sequence, an origin of replication, one or more marker genes, an enhancer element, a promoter and a transcription terminator sequence.

Suitable vectors can be prepared using standard recombinant DNA procedures. Isolated plasmids and DNA fragments are cleaved, tailored, and ligated together in a specific order to generate the desired vectors.

After ligation, the vector with the foreign gene inserted is transformed into a suitable host cell. The transformed cells are selected by growth on an antibiotic, commonly tetracycline (tet) or ampicillin (amp), to which they are rendered resistant due to the presence of tet and/or amp resistance genes on the vector. The transformed cells are grown in culture and the plasmid DNA (plasmid refers to the vector ligated to the foreign gene of interest) is then isolated. This plasmid DNA is then analyzed by restriction mapping and/or DNA sequencing. Methods for DNA sequencing are well-known in the art. See, e.g., Messing et al., Nucleic Acids Res., 9:309 (1981) or by the method of Maxam et al., Methods of Enzymology, 65:499 (1980).

Those of ordinary skill in the art will understand that any genetic manipulations necessary to carry out the present invention, e.g., preparing DNA expression vectors or engineering bacteria, can be carried out using well-known methods and principles in the art. The genetic and/or molecular biology tools required to conduct such manipulations can be any tool well-known in the art, including those acquired from commercial sources, such as from Invitrogen, Inc., Clontech, Inc., BD Biosciences, Promega, Inc., New England Biolabs, Inc., and the like.

Contemplated are glycosylation variants of IL-27, which can be prepared also by techniques well-known in the art. Glycosylation of polypeptides include, but are not limited to, N-linked or O-linked. O-linked glycoslation sites may, for example, be modified by the addition of, or substitution by, one or more serine or threonine residue to the amino acid sequence of a polypeptide. For ease, changes are usually made at the DNA level, essentially using the techniques known for the preparation of amino acid sequence variants.

Also contemplated are IL-27 variants that have chemical or enzymatic couplings to glycosydes, which may also be used to modify or increase the number or profile of carbohydrate substituents. These procedures are advantageous in that they do not require production of the polypeptide that is capable of O-linked (or N-linked) glycosylation. Depending on the coupling mode used, the sugar(s) may be attached to (a) arginine and histidine, (b) free carboxyl groups, (c) free hydroxyl groups such as those of cysteine, (d) free sulfhydryl groups such as those of serine, threonine, or hydroxyproline, (e) aromatic residues such as those of phenylalanine, tyrosine, or tryptophan or (f) the amide group of glutamine. These methods are well-known in the art. See, e.g., WO 87/05330 and Aplin et al., CRC Crit. Rev. Biochem., pp. 259-306 (1981).

The IL-27 variants can also include those having carbohydrate modifications. Chemical deglycosylation requires exposure to trifluoromethanesulfonic acid or an equivalent compound. This treatment results in the cleavage of most or all sugars, except the linking sugar, while leaving the polypeptide intact. Chemical deglycosylation is described by Hakimuddin et al., Arch. Biochem. Biophys. 259, 52 (1987) and by Edge et al., Anal. Biochem. 118, 131 (1981). Carbohydrate moieties can also be removed by a variety of endo- and exoglycosidases as described by Thotakura et al., Meth. Enzymol. 138, 350 (1987). Glycosylation is suppressed by tunicamycin as described by Duskin et al., J. Biol. Chem. 257, 3105 (1982). Tunicamycin blocks the formation of protein-N-glycosydase linkages.

Glycosylated IL-27 variants are also contemplated and can be produced by selecting appropriate host cells. Yeast, for example, introduce glycosylation which varies significantly from that of mammalian systems. Similarly, mammalian cells having a different species (e.g. hamster, murine, insect, porcine, bovine or ovine) or tissue (e.g. lung, liver, lymphoid, mesenchymal or epidermal) origin than the source of the selected variant, can be routinely screened for the ability to introduce variant glycosylation.

The use of covalent derivatives of IL-27 and glycosylation variants are also within the scope hereof. Such modifications can be introduced by reacting targeted amino acid residues of the IL-27 variant with an organic derivatizing agent that is capable of reacting with selected side chains or terminal residues, or by harnessing mechanisms of post-translational modification that function in selected recombinant host cells. Covalent derivatization may be instrumental in turning biologically active IL-27 variants to derivatives which retain the qualitative ability of the corresponding native IL-27 to bind its receptor but are devoid of biological activity, or improve other properties, i.e. half-life, stability, etc. of the molecule. Such modifications are within the ordinary skill in the art and are performed without undue experimentation. Certain post-translational derivatization are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues fall within the scope of the invention. Other post-translational modifications include hydroxylation of proline and lysine, phosphorylation of hydroxyl groups of seryl and threonyl residues, methylation of the alpha-amino groups of lysine, arginine, and histidine side chains [T. E. Creighton, Proteins: Structure and Molecular Properties, W.H. Freeman Co., San Francisco pp. 79-86 (1983)], acetylation of the N-terminal amines and, in some instances, amidation of the C-terminal carboxyl of IL-27.

Other covalent derivatives comprise IL-27 covalently bonded to a nonproteinaceous polymer, such as polyethylene glycol, polypropylene glycol or polyoxyalkylenes, in the manner set forth in U.S. Pat. No. 4,640,835; 4,496,689; 4,301,144; 4,670,417; 4,791,192, 4,179,337, or 5,116,964.

### IL-27 Bioactivity Assays

The present invention also contemplates any suitable analytic technique or tool for use in characterizing the IL-27 polypeptides of the invention, or the biologically active fragments and variants of IL-27.

One assay that can be used to test the bioactivity of IL-27 and its fragments and variants contemplated by the invention is the detection of the phosphorylation level of Stat 1 or Stat3. In another assay, the bioactivity of IL-27 and its fragments and variants contemplated by the invention can be measured by the increased level of IL-10 production. In yet another assay, the bioactivity of IL-27 and its fragments and variants can be measured by an increase in the level of Tbet. Methods for detecting phosphorylation and increased substrate levels are well-known in the art. Such methods include, but are not limited to, immunoassays such as Biacore, FACS analysis, immunofluorescence, immunohistochemical staining, Western blots (immunobots), ELISA, immunoradiometric assays, fluorescent immunoassays, etc.

The bioactivity of the IL-27 of the invention can also be assessed by its affect on the disease state of IBD in animal models. Such animal models are known in the art and have been used for some time in the study of IBD. These are accepted models for understanding the affect of therapeutic agents for treating IBD, including Crohn's Disease and ulcerative colitis. An animal in this model may be administered the IL-27 by any suitable means, as described herein further below, including, for example, by administration of a therapeutically effective amount of a nucleic acid molecule encoding IL-27, the IL-27 polypeptide itself, a microorganism that is genetically engineered to produce the IL-27 in the gastrointestinal tract, or by some other gastrointestinal delivery system (as described further below).

Any suitable animal model of IBD is contemplated by the present invention. For example, the first group of animal models of IBD includes animals spontaneously developing diseases reminiscent of some forms of IBD. Spontaneous animal models include C3H/HeJ mouse, Japanese waltzing mice, swine dysentery and equine colitis, caused by C. difficile, and the cotton top tamarin. The diseases that these animals suffer have recently been subdivided into five types, two of which resemble UC. Of these models, a large proportion of the tamarin animals have some form of gut disorder, and many of them also develop bowel cancer, as do patients with UC, and thus, can be a useful model in the present invention.

In another approach, various irritants, such as ethanol, acetic acid, formalin, immune complexes, trinitrobenzene sulphonic acid (TNBS), dextran sulphate sodium (DSS), bacterial products or carrageenan can be used to generate acute or chronic inflammation. Such a model is described in Morris et al., Gastroenterology 96, 795 (1989).

In yet another approach, transgenic animals can be used to model IBD. Most human patients who have ankylosing spondylitis also carry the gene for HLA-B27. It has been observed that such patients are at greater risk of developing IBD. HLA-B27 transgenic rats, which were generated to model spondyloarthropathies, in addition to the joint disease, also showed symptoms of chronic inflammation of the bowel which, though not identical, had many similarities with CD. Accordingly,the HL/B27 transgenic rats can be used to model IBD.

Another suitable transgenic animal models based on IL-10 “knockout” mice can be used. IL-10 is produced by TH2 cells, stimulates B cells to produce antibody, down-regulates macrophages reducing the production of IL-1, IL-6, IL-8 and TNF-alpha, and shifts the balance of antigen presentation from macrophages to B cells. IL-10 also reduces the production of IFN-gamma, hence reducing the activity of TH1 cells and natural killer cells. Mice treated from birth with anti-IL-10 antibody (given i.p. 3-times weekly) show no changes in body weight or histology of major tissues. The number and proportions of B and T cell lymphocytes are also normal. There is, however, a dramatic reduction in IgA production, whereas the concentrations of IgG2a and IgG-2b are increased. In addition, an almost total depletion of peritoneal B cells, which are a special B cell population carrying the marker Ly-1, was observed. These B cells are continuously derived from bone marrow, have a limited immunoglobulin repertoire which is not subject to somatic mutation, and are responsible for much of the IgM found in plasma. The depletion of these specific B cells may be due to the increased level of IFN-gamma that are produced in the anti-IL-10 antibody-treated mice. This is supported by the observation that if IFN-gamma is given at the same time as the anti-IL-10 antibody, the Ly-1 B cells survive.

Another IBD mouse model system contemplated by the present invention is that developed by Dr. Fiona Powrie (Oxford, UK). In this model, T cells are purified, enriched for high expression of CD45RB, then transferred into Rag 1^{-/-} recipients. Approximately six weeks after T cell transfer, signs of IBD begin to appear. By two months, mice begin to die or must be euthanized and by ten weeks nearly all mice have succumbed. In an embodiment of the invention, as described in the Examples, L. lactis expressing IL-27 (SEQ ID NO: 4), or harboring a control vector, were given daily by oral gavage beginning at six weeks, and resulted in a striking therapeutic benefit. IL-35, another immunosuppressive cytokine, was also cloned into L. lactis and tested in the same experiments. However, IL-35 showed no therapeutic effect.

In addition, the invention contemplates the use of in vitro models of IBD to test the bioactivity of IL-27. An in vitro model of IBD has been developed and in described by Braegger et al. in Chapter 8 of Immunology of Gastrointestinal Disease, MacDonald, T. T. ed, Immunology and Medicine Series, Volume 19, Kluwer Academic Publishers (1992); see also MacDonald et al. Exp. Med. 167:1341-1349 (1988). In this model, small explants (1-2 mm across) of human fetal gut tissue (small or large bowel) containing T lymphocytes at the stage of 15-20 weeks gestation are cultured. The human fetal gut can be maintained in organ culture for several weeks with retention of morphology, epithelial cell renewal and enterocyte function. All of the T cells in the explant can be activated by culturing in the presence of pokeweed mitogen or monoclonal anti-CD3 antibodies. The gross appearance of the explants shows major changes as a result of T cell activation. The changes in the small bowel explants as a result of T cell activation are reminiscent of the mucosal change seen in early stages of Crohn's disease, and the goblet cell depletion seen in colon explants is also a feature of ulcerative colitis. This model can be used to study the interaction of T cells with the gut epithelium, and specifically to observe responses to T cell activation by the presence of the IL-27 of the invention.

Any combination of the foregoing in vitro and in vivo assays can be used to test the bioactivity of IL-27 of the invention.

### IL-27 Compositions

The present invention relates to compositions comprising IL-27 or biologically active fragments or variants thereof (or their encoding nucleic acid molecules) that may be administered to a subject in need thereof (e.g., a person having or may likely have inflammatory bowel disease) for treating IBD, including Crohn's Disease or ulcerative colitis. The present invention contemplates any suitable form, such as compositions comprising nucleic acid molecules encoding IL-27 (or biologically active fragments or variants thereof), compositions comprising IL-27 polypeptides (or biologically active fragments or variants thereof), or compositions comprising recombinant bacteria or other microorganims (eukarotic or prokaryotic) that are engineered to be delivered to the gastrointestinal tract (e.g., by oral ingestion) and to express and release (e.g., by secretion mechanisms) their recominant proteins (e.g., IL-27) to one or more intended tissues of the gastrointestinal tract (e.g., the intestinal mucosa). It will be understood that the particular pharmaceutical composition used may depend upon the administration approach used to deliver the IL-27 or other agents to subjects. Such methods are described in further detail below.

For example, a gene therapy approach is contemplated for administration of IL-27-encoding nucleic acid molecules, which can be used to deliver a suitable nucleic acid molecule to a subject in need which is capable of expressing the encoded recombinant protein of interest (e.g., IL-27) at the site of interest (e.g., in the intestinal tissue), wherein such delivery could be achieved by any suitable means, such as by direct injection at the site of an IBD lesion or affected portion of the colon or small intestine or other area. Any suitable known methods for achieving successful local delivery of such nucleic acid molecules of the invention into the gastrointestinal tract of a subject are contemplated. For example, microsphere delivery systems could be employed to enhance the delivery of the nucleic acid molecules encoding the polypeptide agents of the invention. Microsphere delivery systems include microparticles having a coating that provides localized release of the nucleic acids of the invention into the gastrointestinal tract of the subject (e.g., controlled release formulations such as enteric-coated formulations and colonic formulations). Further details of such methods are indicated below.

Also contemplated are methods and compositions suitable for administering IL-27 polypeptides (or biologically active fragments or variants thereof) or other polypeptide agents of the invention to a subject in need thereof (e.g., a person having or may likely have inflammatory bowel disease) for treating IBD, including Crohn's Disease or ulcerative colitis. Any suitable known methods for achieving successful local delivery of such polypeptides of the invention into the gastrointestinal tract of a subject. For example, microsphere delivery systems such as microparticles having a coating that provides localized release of the polypeptides into the gastrointestinal tract of the subject (e.g., controlled release formulations such as enteric-coated formulations and colonic formulations) can be used. Further details of such methods are indicated below.

### Recombinant Microorganisms

In aspects, the present invention relates to recombinant microorganisms that can express a polypeptide of interest, e.g., IL-27 or its biologically active fragments and variants thereof, to one or more tissues of the gastrointestinal tract (e.g., local secretion at the intestinal mucosa). The microorganism can be any microorganism capable of deliverying recombinant molecules into the gastrointestinal tract of a subject.

In embodiments, the recombinant microorganism is a gram-positive bacterium. In related embodiments, the gram-positive bacterium is non-pathogenic in the sense that it does not cause harm or does not lead to deleterious effects when administered to an intended subject.

In embodiments, the gram-positive bacterium is a lactic acid bacterium (LAB), including, but not limited to the genera Lactococcus, Lactobacillus, Leuconostoc, Pediococcus, Streptococcus, Aerococcus, Carnobacterium, Enterococcus, Oenococcus, Sporolactobacillus, Tetragenococcus, Vagococcus, and Weisella.

In related embodiments, the LAB is a Lactococcus species, such as, but not limited to Lactococcus lactis, Lactococcus garvieae, Lactococcus piscium, Lactococcus plantarum and Lactococcus raffinolactis, and any subspecies and strains thereof. In further embodiments, the LAB is Lactococcus lactis, including any subspecies and strain thereof, such as without limitation Lactococcus lactis ssp. cremoris, Lactococcus lactis ssp. hordniae, Lactococcus lactis ssp. lactis, Lactococcus lactis ssp. bv. diacetylactis. In yet further embodiments, the LAB is Lactococcus lactis ssp. lactis strain CV56 (Genbank accession number CP002365.1), Lactococcus lactis ssp. cremoris strain NZ9000 (Genbank accession number CP002094.1), Lactococcus lactis ssp. lactis strain KF147 (Genbank accession number CP001834.1), Lactococcus lactis ssp. lactis strain IL1403 (Genbank accession number AE005176.1), and Lactococcus lactis ssp. cremoris strain SK11 (Genbank accession number CP000425.1). The sequences of each of these L. lactis species is incorporated herein by reference in their entirety.

In related embodiments, the LAB is an Enterococcus species. In yet further embodiments, the LAB is Enterococcus faecalis, Enterococcus faecium or any subspecies and strains thereof, such as, without limitation Enterococcus faecium strain LMG15709.

The skilled person will appreciate that the IL-27 open reading frames or coding sequences can be coupled to additional sequences that effect a particular purpose. For instance, in order to increase secretion of the exogenous gene, the gene may be coupled to a nucleic acid sequence encoding a secretion signal peptide. In embodiments, the exogenous gene, open reading frame or coding sequence according to the invention is coupled at its 5′ end to the polynucleic acid sequence encoding the Usp45 secretion signal. In related embodiments, the secretion signal originates from a Lactococcus species, e.g., Lactococcus lactis and subspecies and strains thereof. In related embodiments, the secretion signal originates from an Enterococcus species, e.g., Enterococcus faecium and subspecies and strains thereof.

Typically, a secretion signal sequence represents an about 16 to about 35 amino acid segment, usually containing hydrophobic amino acids that become embedded in the lipid bilayer membrane, and thereby allow for the secretion of an accompanying protein or peptide sequence from the host cell. In embodiments, the signal sequence is cleaved from the protein or peptide.

Secretion signal sequences active in suitable host cells are well-known in the art. Exemplary Lactococcus signal sequences include those of usp45 (see, U.S. Pat. No. 5,559,007) and others, see, e.g., Perez-Martinez et al. Mol. Gen. Genet. 234:401-11 (1992); Sibakov et al., Appl. Environ. Microbiol. 57:341-8 (1991). In embodiments, the signal sequence is located between the promoter sequence and the ORF, e.g., the signal sequence is located 3′ from the promoter sequence and precedes the ORF of the polypeptide of interest. In related embodiments, the signal sequence encodes the amino acid sequence MKKKIISAILMSTVILSAAAPLSGVYA (usp45). Alternatively, a mutated usp45 signal sequence (usp45N) may be used, which results in further controllable production and secretion of the polypeptide of interest. The mutant can comprises an asparagine (N) at position 4 instead of a lysine (K), or a K4N mutation. In embodiments, the signal sequence encodes the amino acid sequence MKKNIISAILMSTVILSAAA PLSGVYADTN.

In a further aspect, the invention relates to a replicon comprising the polynucleic acids as described herein. In embodiments, the replicon is a vector, as described herein. In an embodiment, the vector is suitable for prokaryotic expression. In another embodiment, the vector is suitable for homologous recombination in a gram-positive bacterium.

The invention also relates to the use of the gram-positive bacteria according to the invention as described herein for therapy.

Accordingly, in an aspect, the invention relates to the gram-positive bacterium or a pharmaceutical composition comprising the gram-positive bacterium according to the invention as described herein for use as a medicament. In another aspect, the invention relates to the gram-positive bacterium or a pharmaceutical composition comprising the gram-positive bacterium according to the invention as described herein for use in therapy or treatment. In a further aspect, the invention relates to the use of the gram-positive bacterium or a pharmaceutical composition comprising the gram-positive bacterium according to the invention as described herein for the manufacture of a medicament. In yet another aspect, the invention relates to a method of treatment, comprising administering the gram-positive bacterium or a pharmaceutical composition comprising the gram-positive bacterium according to the invention as described herein. In embodiment, the gram-positive bacterium comprises one or more exogenous genes that encodes a product, such as a protein, polypeptide or peptide (e.g., IL-27), which product has a therapeutic or preventive effect in a subject.

The gram-positive bacteria of the present invention may be administered alone or in combination with one or more active compounds. The latter can be administered before, after or simultaneously with the administration of the gram-positive bacteria.

The gram-positive bacteria of the invention can be suspended in a pharmaceutical formulation for administration to the human or animal having the disease to be treated. Such pharmaceutical formulations include but are not limited to live gram-positive bacteria and a medium suitable for administration. The gram-positive bacteria may be lyophilized in the presence of common excipients such as lactose, other sugars, alkaline and/or alkali earth stearate, carbonate and/or sulphate (e.g., magnesium stearate, sodium carbonate and sodium sulphate), kaolin, silica, flavorants and aromas. Gram-positive bacteria so-lyophilized may be prepared in the form of capsules, tablets, granulates and powders (e.g., a mouth rinse powder), each of which may be administered by the oral route. Alternatively, some gram-positive bacteria may be prepared as aqueous suspensions in suitable media, or lyophilized bacteria may be suspended in a suitable medium just prior to use, such medium including the excipients referred to herein and other excipients such as glucose, glycine and sodium saccharinate.

For oral administration, gastroresistant oral dosage forms may be formulated, which dosage forms may also include compounds providing controlled release of the gram-positive bacteria and thereby provide controlled release of the desired protein encoded therein (e.g., IL-27). For example, the oral dosage form (including capsules, tablets, pellets, granulates, powders) may be coated with a thin layer of excipient (e.g., polymers, cellulosic derivatives and/or lipophilic materials) that resists dissolution or disruption in the stomach, but not in the intestine, thereby allowing transit through the stomach in favour of disintegration, dissolution and absorption in the intestine.

The oral dosage form may be designed to allow slow release of the gram-positive bacteria and of the produced exogenous proteins, for instance as controlled release, sustained release, prolonged release, sustained action tablets or capsules. These dosage forms usually contain conventional and well-known excipients, such as lipophilic, polymeric, cellulosic, insoluble, swellable excipients. Such formulations are well-known in the art and are described, for example, in the following references: Hansel et al., Pharmaceutical dosage forms and drug delivery systems, 5th edition, William and Wilkins, 1990; Chien 1992, Novel drug delivery system, 2nd edition, M. Dekker; Prescott et al., Novel drug delivery, J. Wiley & Sons, 1989; and Cazzaniga et al., Int. J. Pharm. i08:7 (1994).

### Administration of IL-27 Compositions

The present invention contemplates any suitable techniques or approaches acceptable in the art for administering IL-27 and the other agents of the invention, including pharmaceutical compositions comprising such agents to a subject in need, e.g., a person having inflammatory bowel disease or a symptom thereof. These administration techniques can include local delivery methods, such as by injection of a IL-27 or nucleic acid molecule encoding IL-27 directly at an IBD lesion or affected portion of the colon, administration of IL-27 in a form that will deliver IL-27 into the gastrointestinal tract in a controlled manner (e.g., microparticle with controlled release coating), or by the administration of a recombinant microorganism engineered to express and secrete the polypeptide agents of the invention to one or more tissues of the gastrointestinal tract (e.g., local secretion at the intestinal mucosa).

In embodiments, the IL-27 or its biologically active fragments and variants thereof or other polypeptide agents of the invention may administered to a subject having or likely to have inflammatory bowel disease via recombinant microorganisms. Although not previously described or suggested for the administration of IL-27, the use of such recombinant microorganisms for the delivery of other recombinant proteins, including cytokines, can be found described, for example, in WO 96/11277, WO 97/14806, WO 00/23471, U.S. Pat. No. 6,746,671, Steidler et al., Infection and Immunity, 66:3183-3189 (1998); and Steidler et al., Science, 289:1352-1355 (2000), each of which are herein incorporated by reference in their entireties.

### Administration of IL-27 by Recombinant Microorganisms

In one aspect, the present invention relates to the preparation and administration of pharmaceutical compositions comprising recombinantly engineered microorganisms, e.g., recombinant L. lactis or E. faecium, for therapeutic treatment of inflammatory bowel disease in animals. The invention further includes methods of administration of such pharmaceutical compositions to a subject having inflammatory bowel disease requiring treatment with the pharmaceutical compositions of the invention.

The invention includes pharmaceutical compositions comprising recombinantly engineered microorganisms, e.g., L. lactis or E. faecium, which are useful for therapeutic treatment of inflammatory bowel disease. This aspect of the invention exploits the ability of certain microorganisms to survive in the mucosal surfaces of animals, which mucosae represent the interface between the exterior and interior regions of the body, and/or undergo sporulation or lyse, thereby releasing the recombinant proteins expressed at the mucosal surfaces, e.g., the intestinal mucosal surface. Once administered to the animal, the recombinant microorganisms of the invention which encode the desired therapeutic protein, e.g., IL-27 or a biologically active fragment or variant thereof, to express and produce the same. The protein so produced then has the desired therapeutic effect either at the site of production, or is selectively transported to the desired anatomical site at which it then exerts the desired therapeutic effect.

In the present invention, microorganisms are manipulated to express desired recombinant proteins using known techniques available in the art for genetically manipulating various types of cells, e.g., bacteria, fungi, and/or yeast cells. Certain properties of bacteria and other microorganisms are exploited in order to render them useful as vehicles for administration of the therapeutic recombinant proteins of the invention, e.g., the IL-27 or its biologically active fragments or variants.

These properties include, but are not limited to, the ability of the microorganisms to adhere to epithelial cells (see Karlsson et al., Ann. Rev. Biochem. 58:309 (1989)); the ability of the microorganisms to sporulate, wherein the spores are resistant to adverse conditions and are capable of producing large quantities of proteins (see Kaiser et al., Cell 73:237 (1993)); and the ability of various microorganisms to have tropism for various tissues and sites of the gastrointestinal tract.

Certain microorganisms are known to possess selective tropism for the mucosa of the intestinal tract, the mucosa of the mouth and esophagus, the mucosa of the nose, pharynx, trachea, the vaginal mucosa, the skin, the eye, and the ear, among other sites. Such a microorganism, e.g., a bacterium, is manipulated so that it comprises a desired gene, which encodes a desired protein useful for treatment of a particular disease state in an animal. The microorganism produces the protein in situ, i.e., inside the animal following delivery, thereby administering the desired protein to the animal.

In addition to comprising the desired gene, the microorganism may also be manipulated to encode other sequence elements that facilitate production of the desired protein by the bacterium or its delivery to a targeted site or tissue or cell. Such sequence elements include, but are not limited to, promoter/regulatory sequences which facilitate constitutive or inducible expression of the protein or which facilitate overexpression of the protein in the bacterium. Additional sequence elements may also include those which facilitate secretion of the protein from the bacterium, accumulation of the protein within the bacterium, and/or programmed lysis of the bacterium in order to release the protein from the same. In addition, targeting sequences may be utilized that enable the desired protein to bind to a particular cell receptor or channel to facilitate the delivery of the protein to a specified cell. Many of the sequence elements referred to above are known to those skilled in the art (see, e.g., Hodgson, Bio/Technology 11:887 (1993), which is incorporated herein by reference).

For example, heat induction, galactose induction, viral promoter induction and heat shock protein induction systems are well described in the art and are readily understood by those skilled in the art. Additional inducible expression systems include gene expression systems which respond to stress, metal ions, other metabolites and catabolites. Other elements which may be useful in the invention will depend upon the type of bacterium to be used, the type of protein to be expressed and the type of target site in the animal. Such elements will be readily apparent to the skilled artisan once armed with the present disclosure.

For example, the present invention further contemplates that the microorganism can be engineered to add, change or enhance its tropism or binding potential with certain cells, tissues or regions of the GI tract. Such modifications can be prepared using known recombinant methods.

This invention includes microorganisms which are capable of producing a pharmacologically active protein, e.g., IL-27 or its biologically active fragments or variants. The pharmacologically active protein may be produced within the microorganism and be released upon lysis of the same. The invention also contemplates that the protein may be excreted or secreted by the microorganism (e.g., through the use of a secretory signal), or may be released by the microorganism upon sporulation, or upon germination of the spore to form a vegetative cell, or upon lysis.

In one embodiment, the IL-27 of the invention can include a leader sequence suitable to allow the polypeptide to be secreted or released from the bacterial delivery system (e.g., the L. lactis system). In certain embodiments, to enable secretion of IL-27, a fragment encoding a secretion leader suitable for use in L. lactis can be added to the 5′ end or 3′ end of the IL-27 sequence of the individual alpha and/or beta chain or of the sequence in which alpha and beta chains are linked. This will result in an IL-27 alpha-beta fusion protein or IL-27 alpha and IL-27 beta fusion proteins with N-terminal or C-terminal secretion leader extensions. This fragment can encode the well-known L. lactis secretion leader from the usp45 gene (van Asseldonk et al., Gene 95:155-160 (1990), which is incorporated herein by reference), having the sequence N-MKKKIISAILMSTVILSAAAPLSGVYA-C, but also any other seretion leader sequence which is functional in the microorganism vehicle used in the invention, e.g., L. lactis or E. faecium.

The types of microorganisms which are useful in the invention include, but are not limited to, yeast, fungi, and bacteria. Fungi suitable for use in the invention include fungal species belonging to any of the fungal genera of Candida, Saccharomyces, Aspergillus or Penicillium. Yeast microorganisms suitable in the invention include, but are not limited to, Hansenula polymorpha, Kluiveromyces lactis, Pichia pastoris, Saccharomyces cerevisiae and Schizosaccharomyces pobe.

Bacterial microorganisms suitable for use in the invention include, but are not limited to, Bacillus subtilis and other suitable sporulating bacteria; members of the genus Bifidobacterium including but not limited to, Bifidobacterium adolescentis, Bifidobacterium angulatum, Bifidobacterium bifidum, Bifidobacterium breve, Bifidobacterium catenulatum, Bifidobacterium infantis, Bifidobacterium longum, and Bifidobacterium pseudocatenulatum; members of the genus Brevibacterium including but not limited to, Brevibacterium epidermis and Brevibacterium lactofermentum; members of the genus Enterobacter including but not limited to, Enterobacter aerogenes, Enterobacter cloacae; members of the genus Enterococcus including but not limited to Enterococcus faecalis; members of the genus Escherichia, including but not limited to, Escherichia coli; members of the genus Lactobacillus including but not limited to, Lactobacillus acidophilus, Lactobacillus amylovorus, Lactobacillus bulgaricus, Lactobacillus brevis, Lactobacillus casei, Lactobacillus crispatus, Lactobacillus curvatus, Lactobacillus delbrueckii, Lactobacillus delbrueckii subspecies bulgaricus, Lactobacillus delbrueckii subspecies lactis, Lactobacillus fermentum, Lactobacillus gasseri, Lactobacillus helveticus Lactobacillus hilgardii, Lactobacillus jensenii, Lactobacillus paracasei, Lactobacillus pentosus, Lactobacillus plantarum, Lactobacillus reuterii, Lactobacillus sake and Lactobacillus vaginalis; members of the genus Lactococcus including but not limited to, Lactococcus lactis, Lactococcus lactis subspecies cremoris and Lactococcus lactis subspecies lactis; members of the genus Propionibacterium including but not limited to Propionibacterium jesenii; members of the genus Staphylococcus including but not limited to, Staphylococcus epidermidis; members of the genus Streptococcus, including but not limited to, Streptococcus lactis, Streptococcus foecalis, Streptococcus gordonii, Streptococcus pyogenes, Streptococcus mutans, Streptococcus thermophilus and Streptococcus salivarius subspecies thermophilus; and members of the genus Enterococcus including but not limited to, Enterococcus faecalis, and Enterococcus faecium.

In some embodiments, the recombinant microorganism is a recombinant microflora species, including a species belonging to the bacterial genera of Bacteriodes, including but not limited to Bacteroides ovatus, Clostridium, Fusobacterium, Eubacterium, Ruminococcus, Peptococcus, Eschericia, or Lactobacillus. In other embodiments, the recombinant microflora species is a fungal species belonging to any of the fungal genera of Candida, Saccharomyces, Aspergillus or Penicillium.

In embodiments, the microorganism of the invention is the bacterium Lactococcus lactis or the bacterium Enterococcus faecium.

Examples of other microorganisms which are useful in the invention include Streptococcus pyogenes, Streptococcus mutans or Streptococcus gordonii, each being capable of colonizing the oral mucosa and expressing and releasing an anti-inflammatory protein capable of ameliorating inflammatory diseases of the gums and teeth.

Similarly, it is possible to exploit the ability of, for example, Escherichia coli, to colonize the intestinal mucosa in order to introduce therapeutic proteins into this region of the body for treatment of intestinal disease including among others for example, ulcerative colitis and Crohn's Disease. Such bacteria may be administered to the animal either orally or rectally. In view of the high absorption capacity of the intestinal mucosa, according to the methods of the invention, expression of recombinant proteins by recombinant bacteria in the intestinal mucosa can result in transport of the produced recombinant protein across the mucosal surface into the bloodstream. Thus, systemic delivery of recombinant proteins is also contemplated by the invention using recombinant microorganisms capable of expressing the same.

It is also possible to use spore-forming bacteria (i.e., Clostridium and Bacillus) which, when in spore form, are naturally resistant to extreme environments and are therefore particularly suitable for oral administration as they are resistant to the effects of gastric acids. Such bacteria, when administered orally to an animal, should reach the intestinal mucosa in an intact, unchanged state. Upon germination, these bacteria then produce the desired active protein in the intestinal mucosa, which protein otherwise may not have survived the effects of the gastric acids.

Spore-forming bacteria may be additionally exploited for their ability to produce spores and thereby deliver proteins to target mucosal sites in the body. In this instance, vegetative state spore-forming bacteria encoding the desired protein are prepared in a formulation suitable for oral or rectal administration. Upon reaching the intestinal mucosa, such organisms are induced to sporulate wherein the vegetative cells lyse thereby releasing the expressed protein into the mucosa. In this manner, a well defined dose of the desired protein is released into the mucosa. Induction of sporulation by bacteria in the intestine or induction of germination of spores is accomplished by further manipulating the genes of these organisms which control such events. Importantly, spore-forming bacteria may be engineered such that they are induced to initiate the process of sporulation but are incapable of forming spores. In this case, the cells containing the desired expressed protein lyse thereby releasing the protein; however, since spores are not in fact formed, no live bacteria remain in the host.

The therapeutic protein, the gene of which is inserted into a suitable expression vector, preferably is non-toxic and non-pathogenic, non-vaccinogenic, i.e., it should not induce a significant immune response which is protective for the host against the protein itself. Further, the therapeutic protein preferably is expressed in an active form or, at least, may be converted into the active form once released by the microorganism. In embodiments, the desired therapeutic protein of the invention is IL-27 or a biologically active fragment of variant thereof. In related embodiments, the IL-27 may be derived from humans and has the amino acid sequence given in SEQ ID NO: 2, which is a chimeric polypeptide comprising the alpha and beta chains of human IL-27 joined by a polypeptide linker of SEQ ID NO: 5. In related embodiments, the IL-27 is derived from mouse and has the amino acid sequence given in SEQ ID NO: 4, which is a chimeric polypeptide comprising the alpha and beta chains of mouse IL-27 joined by a polypeptide linker of SEQ ID NO: 5. In both embodiments, the amino acid sequences are optimized for expression in the desired host recombinant microorganism vehicle, L. lactis or E. faecium.

In other embodiments pertaining to the co-administration of other active agents, a recombinant microorganism strategy is also contemplated as one route to co-administer such additional agents. Such agents can include other proteins, such as other therapeutic cytokines. Examples of additional protein active agents can include, but are not limited to, the following genes: members of the interleukin family of genes, including, but not limited to, IL-2, IL-3, IL-4, IL-5, IL-6, IL-7, IL-8, IL-9, IL-10, IL-11, IL-12, IL-13, IL-14 and IL-15 and genes encoding receptor antagonists thereof; genes which encode hemopoietic growth factors, including, but not limited to, erythropoietin, granulocyte colony stimulating factor, granulocyte macrophage colony stimulating factor, macrophage colony stimulating factor, stem sell factor, leukemia inhibitory factor and thrombopoietin; genes encoding neurotropic factors, including but not limited to, nerve growth factor, brain derived neurotropic factor and ciliary neurotropic factor; and genes which encode interferons, including, but not limited to, IFN-alpha, IFN-beta and IFN-gamma.

Such additional protein active agents can also include: genes encoding chemokines, such as the C—C family and the C—X—C family of cytokines; genes encoding hormones, such as proinsulin and growth hormone; and genes encoding thrombolytic enzymes, including tissue plasminogen activator, streptokinase, urokinase or other enzymes, such as trypsin inhibitor. The invention further includes genes which encode tissue repair factors, growth and regulatory factors such as, but not limited to, oncostatine M, platelet-derived growth factors, fibroblast growth factors, epidermal growth factor, hepatocyte growth factor, bone morphogenic proteins, insulin-like growth factors, calcitonin and transforming growth factor alpha and beta.

It is well-known that proteins which are active only in glycosylated form must be expressed in microorganisms such as yeast. Thus, the invention contemplates that in such cases, such proteins may be administered using yeast-based recombinant microorganism vehicles. In other embodiments, including the use of IL-27, the encoded proteins can be active in a non-glycosylated form so that they can be expressed in bacterial delivery vehicles, such as L. lactis, B. subtilis, or E. coli.

The recombinant microorganisms of the invention, including, for example, the recombinant L. lactis engineered to express IL-27, can be suspended in any suitable pharmaceutically acceptable formulation for administration to the human or animal having the disease to be treated, e.g., inflammatory bowel disease, including Crohn's or ulcerative colitis.

Such pharmaceutical formulations can include live microorganisms and a pharmaceutically acceptable carrier suitable for administration. The recombinant microorganisms may be lyophilized in the presence of common excipients such as lactose, other sugars, alkaline and/or alkali earth stearate, carbonate and/or sulfate (for example, magnesium stearate, sodium carbonate and sodium sulfate), kaolin, silica, flavorantε and aromas. Microorganisms so lyophilized may be prepared in the form of capsules, tablets, granulates and powders, each of which may be administered by the oral route. Alternatively, some recombinant bacteria, or even spores thereof, may be prepared as aqueous suspensions in a suitable medium, or lyophilized bacteria or spores may be suspended in a suitable medium just prior to use, such medium including the excipients referred to herein and other excipients such as glucose, glycine and sodium saccharinate, or any other suitable medium known to those of ordinary skill in the art.

For oral administration, gastroresistant oral dosage forms may be formulated, which dosage forms may also include compounds providing controlled release of the microorganisms and thereby provide controlled release of the desired protein encoded therein. For example, the oral dosage form (including tablets, pellets, granulates, powders) may be coated with a thin layer of excipient (usually polymers, cellulosic derivatives and/or lipophilic materials) that resists dissolution or disruption in the stomach, but not in the intestine, thereby allowing transit through the stomach in favour of disintegration, dissolution and absorption in the intestine. The oral dosage form may be designed to allow slow release of the microorganism and of the recombinant protein thereof, for instance, as controlled release, sustained release, prolonged release, sustained action tablets or capsules.

These dosage forms can contain conventional and well-known excipients, such as lipophilic, polymeric, cellulosic, insoluble, swellable excipients. When the compositions of the invention are to be administered rectally, pharmaceutical formulations may include suppositories and creams. In this instance, the microorganisms can be suspended in a mixture of common excipients including lipids.

Each of the aforementioned formulations are well-known in the art and are described, for example, in Hansel et al.; Chien; and Cazzaniga et al. Thus, according the invention, recombinant microorganisms encoding a desired gene may be administered to the animal or human via any suitable route, e.g., oral.

Dosages of microorganisms for administration will vary depending upon any number of factors including the type of bacteria and the gene encoded thereby, the type and severity of the disease to be treated and the route of administration to be used. Thus, precise dosages cannot be defined for each and every embodiment of the invention, but will be readily apparent to those skilled in the art once armed with the present invention. For example, the dosage could be determined in a case by case way by measuring the serum level concentrations of the recombinant protein after administration of predetermined numbers of cells, using well-known methods, such as those known as ELISA or using a Biacore system (GE Healthcare, the contents of any product manuals or literature of which are incorporated by reference). The analysis of the kinetic profile and half life of the delivered recombinant protein provides sufficient information to allow the determination of an effective dosage range for the transformed microorganisms. As an example, L. lactis encoding IL-27 may be administered to an animal at a dose of approximately 10⁹ colony forming units (cfu)/kg body weight/day, or even up to 10¹⁰, 10¹¹, or 10¹² colony forming units (cfu)/kg body weight/day.

The pharmaceutical compositions comprising the recombinant microorganisms of the invention can also be, in certain other embodiments, delivered locally to a specific site (e.g., a Crohn's Disease lesion or IBD-related sited of inflammation) in the gastrointestinal tract by any suitable non-invasive technology, including, for example, a catheter systems, a colonoscope, an endoscope, or other similar means for delivering a pharmaceutical composition of the invention. Such technologies are well-known in the art and can be found further described, for example, in: U.S. Pat. No. 7,591,783; 7,582,055; 7,578,786; 7,544,163; 7,530,948; 7,448,995; 7,413,543; 7,258,663; 7,235,045; 7,229,407; 7,074,181; 7,042,488; 6,974,411; 6,902,527; 6,869,397; 6,537,211; 6,425,535; 5,746,692; 5,704,899; 5,170,774; 5,110,645; 4,946,442; 4,857,057; or 3,941,121, each of which is incorporated herein by reference. In embodiments, the invention provides for the local delivery of L. lactis or E. faecium engineered to express and secrete IL-27 or a biologically active fragment or variant thereof to a tissue or site of the gastrointestinal tract, including, for example, lesions of Crohn's Disease or inflammation site, by utilizing a catheter delivery system that allows the pharmaceutical composition to be deposited within the interior of the colon or other site directly at a site for treatment.

### Administration of IL-27 Polypeptides or Nucleic Acid Molecules

The present invention contemplates the administration of IL-27 or any bioactive fragments or variants thereof and/or any nucleic acid molecule encoding same by any suitable route for local delivery to the gastrointestinal tract, e.g., oral controlled delivery, direct delivery to lesion, or direct delivery to site of inflammation. Methods and techniques for preparing suitable delivery systems for IL-27 polypeptides and IL-27 encoding nucleic acid molecules are well-known in the art, and are described in part below.

Lipid based microsphere delivery systems can be used to delivery the polypeptides and/or the nucleic acid molecules of the invention. Optionally, such systems can be modified such that they specifically target the cells and/or tissues of the gastrointestinal tract. Methods for preparing such systems will be well-known to those having ordinary skill in the art. For example, the microspheres comprising the IL-27 or IL-27-encoding nucleic acid molecules can be modified to comprise one or more ligands or targeting moieties which allow the microsphere to bind and/or interact specifically with a receptor or other target on a target cell or tissue of the gastrointestinal tract.

Accordingly, in one aspect, the present invention provides IL-27 or IL-27-encoding nucleic acid formulations comprised of a lipid-based carrier system, such as a stabilized nucleic acid-lipid particle, cationic lipid or liposome nucleic acid complexes (i.e., lipoplexes), a liposome, a micelle, a virosome, or a mixture thereof, which optionally may be modified to contain a moiety that enables it to be targeted to one or more cells or tissues of the gastrointestinal tract. In other embodiments, the carrier system is a polymer-based carrier system such as a cationic polymer-nucleic acid complex (i.e., polyplex), which optionally may be modified to contain a moiety that enables it to be targeted to one or more cells or tissues of the gastrointestinal tract. In additional embodiments, the carrier system is a cyclodextrin-based carrier system, such as a cyclodextrin polymer-nucleic acid complex, which optionally may be modified to contain a moiety that enables it to be targeted to one or more cells or tissues of the gastrointestinal tract. In further embodiments, the carrier system is a protein-based carrier system such as a cationic peptide-nucleic acid complex. Nucleic acid-lipid and/or protein-lipid particles and their method of preparation are disclosed in, e.g., U.S. Pat. Nos. 5,753,613; 5,785,992; 5,705,385; 5,976,567; 5,981,501; 6,110,745; and 6,320,017; and PCT Publication No. WO 96/40964, which are all herein incorporated by reference.

The lipoplexes of the invention can include non-cationic lipids used in the formulations of the present invention, which include any of a variety of neutral uncharged, zwitterionic, or anionic lipids capable of producing a stable complex. Such non-cationic lipids can be neutral or negatively charged. Examples of non-cationic lipids include, without limitation, phospholipid-related materials such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipahnitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), and stearoyloleoyl-phosphatidylethanolamine (SOPE). Non-cationic lipids or sterols such as cholesterol may also be present. Additional nonphosphorous containing lipids include, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, diacylphosphatidylcholine, diacylphosphatidylethanolamine, and the like. Other lipids such as lysophosphatidylcholine and lysophosphatidylethanolamine may be present. Non-cationic lipids also include polyethylene glycol (PEG)-based polymers such as PEG 2000, PEG 5000, and polyethylene glycol conjugated to phospholipids or to ceramides (referred to as PEG-Cer), as described in U.S. patent application Ser. No. 08/316, 429.

In certain embodiments, the non-cationic lipids are diacylphosphatidylcholine (e.g., distearoylphosphatidylcholine, dioleoylphosphatidylcholine, dipalmitoylphosphatidylcholine, and dilinoleoylphosphatidylcholine), diacylphosphatidylethanolamine (e.g., dioleoylphosphatidylethanolamine and palmitoyloleoyl-phosphatidylethanolamine), ceramide, or sphingomyelin.

A cationic lipid of a formulation of the instant invention may be, e.g., N,N-dioleyl-N, N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxypropylamine (DODMA), 1,2-DiLinoleyloxy-N,N-dimethylaminopropane (DLinDMA), 1,2-Dilinolenyloxy-N,N-dimethylaminopropane (DLendMA), DSDMA, DOSPA, DOGS, DC-Chol, DMRIE or mixtures thereof.

A number of these lipids and related analogs have been described in U.S. Patent Publication No. 20060083780; U.S. Pat. Nos. 5,208,036; 5,264,618; 5,279,833; 5,283,185; 5,753,613; and 5,785,992; and PCT Publication No. WO 96/10390. Additionally, a number of commercial preparations of cationic lipids for DNA/RNA delivery are available and can be used in the present invention. These include, for example, LIPOFECTIN® (commercially available cationic liposomes comprising DOTMA and DOPE, from GEBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA).

The formulations of the instant invention may further comprise cholesterol. If present, the cholesterol typically comprises from about 0 mol % to about 10 mol %, from about 2 mol % to about 10 mol %, from about 10 mol % to about 60 mol %, from about 12 mol % to about 58 mol %, from about 20 mol % to about 55 mol %, from about 30 mol % to about 50 mol %, or about 48 mol % of the total lipid present in the formulation.

Conjugated lipids may also be included in the formulations of the invention, including a polyethyleneglycol (PEG)-lipid conjugate, a polyamide (ATTA)-lipid conjugate, a cationic-polymer-lipid conjugate (CPL), or mixtures thereof. In certain embodiments, a nucleic acid-lipid formulation of the invention comprises either a PEG-lipid conjugate or an ATTA-lipid conjugate. Optionally, a PEG-lipid conjugate or ATTA-lipid conjugate is used together with a CPL. A conjugated lipid of a formulation of the invention may comprise a PEG-lipid including, e.g., a PEG-diacylglycerol (DAG), a PEG dialkyloxypropyl (DAA), a PEG-phospholipid, a PEG-ceramide (Cer), or mixtures thereof. A PEG-DAA conjugate may be a PEG-dilauryloxypropyl (C12), a PEG-dimyristyloxypropyl (C14), a PEG-dipalmityloxypropyl (C16), or a PEG-distearyloxypropyl (C18). Optionally, a conjugated lipid is a CPL that has the formula: A-W-Y, wherein A is a lipid moiety, W is a hydrophilic polymer, and Y is a polycationic moiety. W may be a polymer selected from the group consisting of PEG, polyamide, polylactic acid, polyglycolic acid, polylactic acid/polyglycolic acid copolymers, or combinations thereof, the polymer having a molecular weight of from about 250 to about 7000 daltons. In some embodiments, Y has at least 4 positive charges at a selected pH. In some embodiments, Y may be lysine, arginine, asparagine, glutamine, derivatives thereof, or combinations thereof. In certain embodiments, a conjugated lipid is present in a formulation of the instant invention from 0 mol % to about 20 mol % or about 2 mol % of the total lipid present in the formulation.

In addition to cationic and non-cationic lipids, a formulation of the present invention can comprise a stabilizing component (SC) such as an ATTA-lipid or a PEG-lipid such as PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides, or a mixture thereof (see, e.g., U.S. Pat. No. 5,885,613). In certain embodiments, the SC is a conjugated lipid that prevents the aggregation of formulation particles. Suitable conjugated lipids include, but are not limited to, PEG-lipid conjugates, ATTA-lipid conjugates, cationic-polymer-lipid conjugates (CPLs), and mixtures thereof. In additional embodiments, formulation particles comprise either a PEG-lipid conjugate or an ATTA-lipid conjugate together with a CPL.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). In addition, monomethoxypolyethyleneglycol-acetic acid (MePEG-CH₂COOH) is particularly useful for preparing PEG-lipid conjugates including, e.g., PEG-DAA conjugates.

In certain embodiments, a PEG used in a formulation of the invention has an average molecular weight of from about 550 daltons to about 10,000 daltons, optionally from about 750 daltons to about 5,000 daltons, optionally from about 1,000 daltons to about 5,000 daltons, optionally from about 1,500 daltons to about 3,000 daltons, and optionally about 2, 000 daltons or about 750 daltons. The PEG can be optionally substituted by an alkyl, alkoxy, acyl, or aryl group. The PEG can be conjugated directly to the lipid or may be linked to the lipid via a linker moiety. A linker moiety suitable for coupling the PEG to a lipid can be used including, e.g., non-ester containing linker moieties and ester-containing linker moieties.

Phosphatidylethanolamines having a variety of acyl chain groups of varying chain lengths and degrees of saturation can be conjugated to PEG to form a stabilizing component. Such phosphatidylethanolamines are commercially available, or can be isolated or synthesized using conventional techniques known to those of skilled in the art. Exemplary phosphatidylethanolamines contain saturated or unsaturated fatty acids with carbon chain lengths in the range of C₁₀ to C₂₀. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

In addition to the foregoing components, formulation particles or lipoplexes (comprising the IL-27 or IL-27-encoding nucleic acids of the invention) of the present invention can further comprise cationic poly(ethylene glycol) (PEG) lipids or CPLs that have been designed for insertion into lipid bilayers to impart a positive charge (see, e.g., Chen et al., Bioconj. Chem., 11:433-437 (2000)). Exemplary SPLPs and SPLP-CPLs that can be used in the formulations of the instant invention, and methods of making and using SPLPs and SPLP-CPLs, are disclosed, e.g., in U.S. Pat. No. 6,852,334 and PCT Publication No. WO 00/62813. Cationic polymer lipids (CPLs) which may also be used in the formulations of the instant invention in the present invention have the following architectural features: (1) a lipid anchor, such as a hydrophobic lipid, for incorporating the CPLs into the lipid bilayer; (2) a hydrophilic spacer, such as a polyethylene glycol, for linking the lipid anchor to a cationic head group; and (3) a polycationic moiety, such as a naturally occurring amino acid, to produce a protonizable cationic head group.

As mentioned above, in certain instances, the lipoplex formulations of the invention comprise a ligand, such as a targeting ligand for binding to a specific target cell or tissue of the gastrointestinal tract. In certain instances, the ligand of the formulation has a positive charge. Exemplary ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

Non-limiting examples of additional lipid-based carrier systems suitable for use in the present invention include lipoplexes (see, e.g., U.S. Patent Publication No. 20030203865; and Zhang et al., J. Control Release, 100:165-180 (2004)), pH-sensitive lipoplexes (see, e.g., U.S. Patent Publication No. 2002/0192275), reversibly masked lipoplexes (see, e.g., U.S. Patent Publication Nos. 2003/0180950), cationic lipid-based compositions (see, e.g., U.S. Pat. No. 6,756,054; and U.S. Patent Publication No. 2005/0234232), cationic liposomes (see, e.g., U.S. Patent Publication Nos. 2003/0229040, 2002/0160038, and 2002/0012998; U.S. Pat. No. 5,908,635; and PCT Publication No. WO 01/72283), anionic liposomes (see, e.g., U.S. Patent Publication No. 2003/0026831), pH-sensitive liposomes (see, e.g., U.S. Patent Publication No. 2002/0192274; and AU 2003/210303), antibody-coated liposomes (see, e.g., U.S. Patent Publication No. 2003/0108597; and PCT Publication No. WO 00/50008), cell-type specific liposomes (see, e.g., U.S. Patent Publication No. 2003/0198664), liposomes containing nucleic acid and peptides (see, e.g., U.S. Pat. No. 6,207,456), liposomes containing lipids derivatized with releasable hydrophilic polymers (see, e.g., U.S. Patent Publication No. 2003/0031704), lipid-entrapped nucleic acid (see, e.g., PCT Publication Nos. WO 03/057190 and WO 03/059322), lipid-encapsulated nucleic acid (see, e.g., U.S. Patent Publication No. 2003/0129221; and U.S. Pat. No. 5,756,122), other liposomal compositions (see, e.g., U.S. Patent Publication Nos. 2003/0035829 and 2003/0072794; and U.S. Pat. No. 6,200,599), stabilized mixtures of liposomes and emulsions (see, e.g., EP1304160), emulsion compositions (see, e.g., U.S. Pat. No. 6,747,014), and nucleic acid micro-emulsions (see, e.g., U.S. Patent Publication No. 2005/0037086).

Examples of polymer-based carrier systems suitable for use in the present invention include, but are not limited to, cationic polymer-nucleic acid complexes (i.e., polyplexes). To form a polyplex, cargo (e.g., IL-27 or a nucleic acid encoding IL-27) is typically complexed with a cationic polymer having a linear, branched, star, or dendritic polymeric structure that condenses the cargo into positively charged particles capable of interacting with anionic proteoglycans at the cell surface and entering cells by endocytosis. In some embodiments, the polyplex comprises nucleic acid complexed with a cationic polymer such as polyethylenimine (PEI) (see, e.g., U.S. Pat. No. 6,013,240; commercially available from Qiagen, Inc. (Carlsbad, Calif.) as In vivo jetPEI®, a linear form of PEI), polypropylenimine (PPI), polyvinylpyrrolidone (PVP), poly-L-lysine (PLL), diethylaminoethyl (DEAE)-dextran, poly(β-amino ester) (PAE) polymers (see, e.g., Lynn et al., J. Am. Chem. Soc., 123:8155-8156 (2001)), chitosan, polyamidoamine (PAMAM) dendrimers (see, e.g., Kukowska-Latallo et al., Proc. Natl. Acad. Sci. USA, 93:4897-4902 (1996)), porphyrin (see, e.g., U.S. Pat. No. 6,620,805), polyvinylether (see, e.g., U.S. Patent Publication No. 20040156909), polycyclic amidinium (see, e.g., U.S. Patent Publication No. 20030220289), other polymers comprising primary amine, imine, guanidine, and/or imidazole groups (see, e.g., U.S. Pat. No. 6,013,240; PCT Publication No. WO/9602655; PCT Publication No. WO95/21931; Zhang et al., J. Control Release, 100:165-180 (2004); and Tiera et al., Curr. Gene Ther., 6:59-71 (2006)), and a mixture thereof. In other embodiments, the polyplex comprises cationic polymer-nucleic acid complexes as described in U.S. Patent Publication Nos. 2006/0211643, 2005/0222064, 2003/0125281, and 2003/0185890, and PCT Publication No. WO 03/066069; biodegradable poly(β-amino ester) polymer-nucleic acid complexes as described in U.S. Patent Publication No. 2004/0071654; microparticles containing polymeric matrices as described in U.S. Patent Publication No. 2004/0142475; other microparticle compositions as described in U.S. Patent Publication No. 2003/0157030; condensed nucleic acid complexes as described in U.S. Patent Publication No. 2005/0123600; and nanocapsule and microcapsule compositions as described in AU 2002358514 and PCT Publication No. WO 02/096551.

In certain instances, the cargo (e.g., IL-27 or IL-27-encoding DNA) may be complexed with cyclodextrin or a polymer thereof. Non-limiting examples of cyclodextrin-based carrier systems include the cyclodextrin-modified polymer-nucleic acid complexes described in U.S. Patent Publication No. 2004/0087024; the linear cyclodextrin copolymer-nucleic acid complexes described in U.S. Pat. Nos. 6,509,323, 6,884,789, and 7,091,192; and the cyclodextrin polymer-complexing agent-nucleic acid complexes described in U.S. Pat. No. 7,018,609. In certain other instances, the cargo (e.g., a nucleic acid such as a DsiRNA) may be complexed with a peptide or polypeptide. An example of a protein-based carrier system includes, but is not limited to, the cationic oligopeptide-nucleic acid complex described in PCT Publication No. WO95/21931.

### Administration of IL-27 by Other Gastrointestinal Delivery Systems

Any suitable gastrointestinal delivery system known in the art or previously described may be utilized or modified and used to deliver the IL-27 polypeptides and/or nucleic acid molecules and/or the lipid-based formulations and/or the recombinant microorganism delivery systems of the invention to the affected regions or sites of the gastrointestinal tract of subjects having inflammatory bowel disease.

For example, U.S. Pat. No. 6,531,152 describes a gastrointestinal delivery system having a swellable core material that is surrounded by a water-insoluble or relatively water-insoluble coating material in which particulate water-insoluble material is embedded and in which the active agent of interest is contained. When the delivery device enters the gastrointestinal tract, the particulate matter takes up liquid, thus forming channels interconnecting the drug-containing core with the outside of the delivery device. Through these channels liquid enters the core which then swells to the point at which the coating is broken. When the integrity of the coating is destroyed, the core then disintegrates immediately releasing all or most of the drug at a specific site. By controlling parameters in the device, such as the core material, carrier material in the coating, and particulate matter, the location of release of the drug can be carefully controlled. Such a system can be used to deliver the IL-27 or an nucleic acid encoding the IL-27 to the gastrointestinal tract in a location- and time-dependent manner.

U.S. Pat. No. 5,686,105 and U.S. Pat. No. 5,686,106 (both to Kelm, G. R.) describe the use of polymers to coat an active agent for delivery to the colon. The polymers dissolve at about the time that the dosage form reaches the inlet between the small intestine and the colon, or thereafter in the colon. Examples of such polymers include cellulose acetate phthalate. Such a system could be employed to administer the IL-27 or a nucleic acid molecule encoding IL-27 of the invention to a local disease-affected site in the gastrointestinal tract.

U.S. Pat. No. 5,464,633 (Conte, U., et al.) describes a tablet that consists of a core containing the active substance, and an external layer that is able to prevent the immediate release of the active substance. The external layer can be a natural and/or synthetic polymeric substance in the class of the erodible and/or gellable and/or soluble in an aqueous medium hydrophilic polymers and adjuvant substances. Lastly, the layer is surrounded by a gastroresistant and enterosoluble coating. Such a system could be employed to administer the IL-27 or a nucleic acid molecule encoding IL-27 of the invention to a local disease-affected site in the gastrointestinal tract.

Systems involving biomaterials such as chitosan could also be employed to deliver the IL-27 of the invention. See, e.g., Borchard et al., Adv. Drug Deliv Rev. 52145-150 (2001); and Lee et al., Pharm. Res. 18:427-431 (2001). Other systems for oral delivery of drugs can be used and found described in the art, including, for example, in Novel Drug Delivery Systems, Ch. 3, Oral Drug Delivery and Delivery Systems, Ed. Yie W. Chien, 2^{nd} Edition, Drugs and Pharmaceutical Sciences, Vol. 50, New York, 1992, the contents of which are incorporated by reference.

Methods, mechanisms, and formulations for controlling delivery throughout the gastrointestinal tract are well-known in the art. See Singh, Recent Pat. Drug Deliv. Formul. 1:53-63 (2007), the entire contents of which are incorporated herein by reference. As such, one of skill in the art will be readily able prepare an appropriate formulation and control the release of the active substance throughout the gastrointestinal tract. For example, release of the active substance into the small intestine can be achieved by using an enteric polymeric coating (e.g., a coating that is stable at the highly acidic pH found in the stomach, but dissolves in the alkaline environment of the small intestine). See, e.g., U.S. Patent Pub. Nos. 20030152627 and 20030152627, which are hereby incorporated by reference. Coating technology can also be employed to cause release of the active substance into the colon. Such formulations may rely on a pH-dependent mechanism or can be delayed-release formulations. In any of the aforementioned delivery systems, the delivery system can also be a sustained release delivery system. One of ordinary skill in the art is readily able to modify the formulation based on the desired target site for delivery.

Still other gastrointestinal delivery systems in the scope of the invention can include, for example, those described in U.S. Pat. Nos. 5,840,332, 6,949,258, 6,214,378, 6,451,345 and WO/2008/068584, each of which is hereby incorporated by reference.

### Methods of Treatment

As described in detail herein, local delivery of IL-27, e.g., recombinant microorganism delivery systems or microparticles that provide controlled delivery of IL-27 into the gastrointestinal tract, effectively treats inflammatory bowel disease.

As such, the invention includes methods for treating inflammatory bowel disease, mucosal inflammatory pathology or intestinal inflammatory pathology in a subject in need thereof.

In another aspect, of the invention includes methods for treating a condition sensitive to IL-27 in a subject in need thereof.

In any of the methods described herein, some embodiments include locally administering to the intestinal mucosa of the subject a therapeutically effective amount of IL-27 or a therapeutic variant or fragment thereof.

In related embodiments, the IL-27 is administered using a gastrointestinal delivery system.

In some embodiments, the gastrointestinal delivery system is a recombinant microorganism effective to produce the IL-27 in situ in the intestinal mucosa in the subject. In embodiments, the recombinant microorganism is a microflora species, including but not limited to, bacteria, yeast, and fungus.

Exemplary bacteria include, but are not limited to, bacteria from the genera Bacteriodes, Clostridium, Fusobacterium, Eubacterium, Ruminococcus, Peptococcus, Eschericia, Lactobacillus, Enterococcus or Lactococcus. In embodiments, the bacteria is a gram positive bacteria. In related embodiments, the bacteria is Lactococcus lactis or Enterococcus faecium. Examples of Lactococcus lactis include Lactococcus lactis ssp. cremoris SK11, Lactococcus lactis ssp. cremoris MG1363, or Lactococcus lactis ssp lactis IL1403.

Exemplary fungi include, but are not limited to, fungi from the genera Candida, Saccharomyces, Aspergillus or Penicillium.

Exemplary yeast include, but are not limited to, yeast from the genera Hansenula, Kluiveromyces, Pichia, Saccharomyces and Schizosaccharomyces.

In other embodiments, the gastrointestinal delivery system is a microparticle containing IL-27. In related embodiments, the microparticle further contains a coating that enables controlled release of the IL-27 into the gastrointestinal tract. The coating may also enable continuous or sustained release of the IL-27 into the gastrointestinal tract.

In embodiments, the inflammatory bowel disease is Crohn's Disease.

In embodiments, the inflammatory bowel disease is ulcerative colitis.

In embodiments, the condition is colon cancer or another cancer of a tissue of the gastrointestinal tract.

In embodiments, the condition is an inflammatory condition in a tissue of the gastrointestinal tract, including inflammation of the intestine, stomach, liver, pancreas or peritoneum.

In embodiments, the therapeutically effective amount of the IL-27 is sufficient to reduce the non-specific inflammation in the gastrointestinal tract by at least 10-99%. In related embodiments, the therapeutically effective amount of the IL-27 is sufficient to reduce the non-specific inflammation in the gastrointestinal tract by at least 10-25%, 25-50%, 10-50%, 50-90%, 50-75%, 50-70%, 50-80%, 50-90%, 60-70%, 60-80%, 60-90%, 70-80%, 80-90%, 90-95%, 90-99%, or 95-99%.

In another aspect of the invention, any of the methods described herein further involve administering a second therapeutic agent. In embodiments, the second therapeutic agent is a corticosteroid, sulphasalazine, derivative of sulphasalazine, immunosuppressive drug, cyclosporin A, mercaptopurine, azathioprine, cytokine or cytokine antagonist. In related embodiments, the cytokine or cytokine antagonist is tumor necrosis factor-α antagonist, IL-10, IL-27, or IL-35. In other embodiments, the second therapeutic agent is administered intravenously, parenterally, orally or transdermally. In related embodiments, the second therapeutic agent is delivered by a recombinant microorganism.

### Kits

In another aspect, the invention provides kits for use in administering the IL-27 or IL-27-encoding nucleic acid molecules of the invention or the recombinant microorganism delivery systems of the invention for treating inflammatory bowel disease.

Depending on how the kit is to be operated, the kit may include a recombinant microorganism host cell which is to be engineered or has already been engineered to express the IL-27 of the invention (or a biologically active fragment or variant thereof). The particular recombinant microorganism host can include, for example, any suitable yeast, fungus, or bacteria. Fungi suitable for use in the invention include, but are not limited to, fungal species belonging to any of the fungal genera of Candida, Saccharomyces, Aspergillus or Penicillium. Yeast microorganisms suitable in the invention include, but are not limited to, Hansenula polymorpha, Kluiveromyces lactis, Pichia pastoris, Saccharomyces cerevisiae and Schizosaccharomyces pobe.

Bacterial microorganisms suitable for use in the invention include, but are not limited to, Bacillus subtilis and other suitable sporulating bacteria; members of the genus Bifidobacterium including but not limited to, Bifidobacterium adolescentis, Bifidobacterium angulatum, Bifidobacterium bifidum, Bifidobacterium breve, Bifidobacterium catenulatum, Bifidobacterium infantis, Bifidobacterium longum, and Bifidobacterium pseudocatenulatum; members of the genus Brevibacterium including but not limited to, Brevibacterium epidermis and Brevibacterium lactofermentum; members of the genus Enterobacter including but not limited to, Enterobacter aerogenes, Enterobacter cloacae; members of the genus Enterococcus including but not limited to Enterococcus faecalis and Enterococcus faecium; members of the genus Escherichia, including but not limited to, Escherichia coli; members of the genus Lactobacillus including but not limited to, Lactobacillus acidophilus, Lactobacillus amylovorus, Lactobacillus bulgaricus, Lactobacillus brevis, Lactobacillus casei, Lactobacillus crispatus, Lactobacillus curvatus, Lactobacillus delbrueckii, Lactobacillus delbrueckii subspecies bulgaricus, Lactobacillus delbrueckii subspecies lactis, Lactobacillus fermentum, Lactobacillus gasseri, Lactobacillus helveticus Lactobacillus hilgardii, Lactobacillus jensenii, Lactobacillus paracasei, Lactobacillus pentosus, Lactobacillus plantarum, Lactobacillus reuterii, Lactobacillus sake and Lactobacillus vaginalis; members of the genus Lactococcus including but not limited to, Lactococcus lactis, Lactococcus lactis subspecies cremoris and Lactococcus lactis subspecies lactis; members of the genus Propionibacterium including but not limited to Propionibacterium jesenii; members of the genus Staphylococcus including but not limited to, Staphylococcus epidermidis; members of the genus Streptococcus, including but not limited to, Streptococcus lactis, Streptococcus foecalis, Streptococcus gordonii, Streptococcus pyogenes, Streptococcus mutans, Streptococcus thermophilus and Streptococcus salivarius subspecies thermophilus.

In embodiments, the recombinant microorganism is a recombinant microflora species, including a species belonging to the bacterial genera of Bacteroides, including but not limited to Bacteroides ovatus, Clostridium, Fusobacterium, Eubacterium, Ruminococcus, Peptococcus, Eschericia, or Lactobacillus.

In embodiments, the microorganism of the invention is the bacterium Lactococcus lactis or Enterococcus faecium.

Examples of other microorganisms which are useful in the invention include Streptococcus pyogenes, Streptococcus mutans or Streptococcus gordonii, each being capable of colonizing the oral mucosa and expressing and releasing an anti-inflammatory protein capable of ameliorating inflammatory diseases of the gums and teeth.

In embodiments, the kits may also include an immunodetection reagent or label for the detection of the expression of the IL-27 by the recombinant microorganism host. Suitable detection reagents are well-known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immunodetection reagents and processes suitable for application in connection with the novel methods of the present invention are generally well-known in the art.

The reagents may also include ancillary agents such as buffering agents and protein stabilizing agents, e.g., polysaccharides and the like. The diagnostic kit may further include, where necessary, agents for reducing background interference in a test, agents for increasing signal, apparatus for conducting a test, calibration curves and charts, standardization curves and charts, and the like.

In a further embodiment, such a kit can comprise instructions for suitable operational parameters in the form of a label or separate insert.

The above disclosure generally describes the present invention. A more complete understanding can be obtained by reference to the following specific examples, which are provided for purposes of illustration only and are not intended to limit the scope of the invention.

### EXAMPLES

The materials, compositions, and methods described herein are intended to be representative examples of the invention, and it will be understood that the scope of the invention is not limited by the scope of the examples. Those skilled in the art will recognize that the invention may be practiced with variations on the disclosed materials, compositions and methods, and such variations are regarded as within the ambit of the invention.

### Example 1

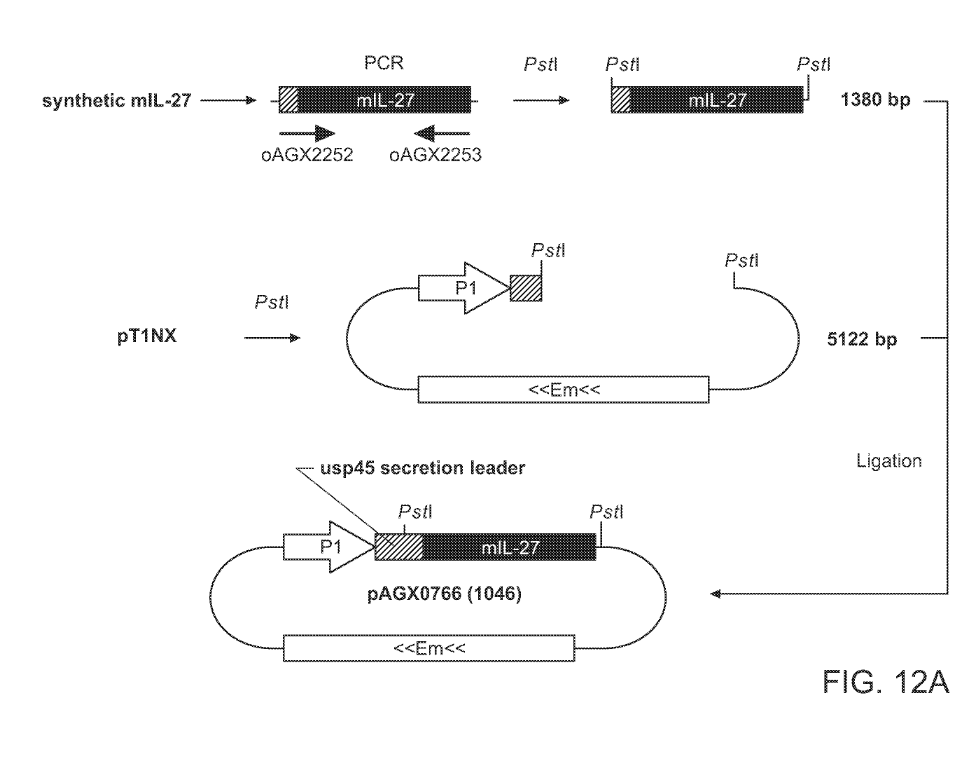

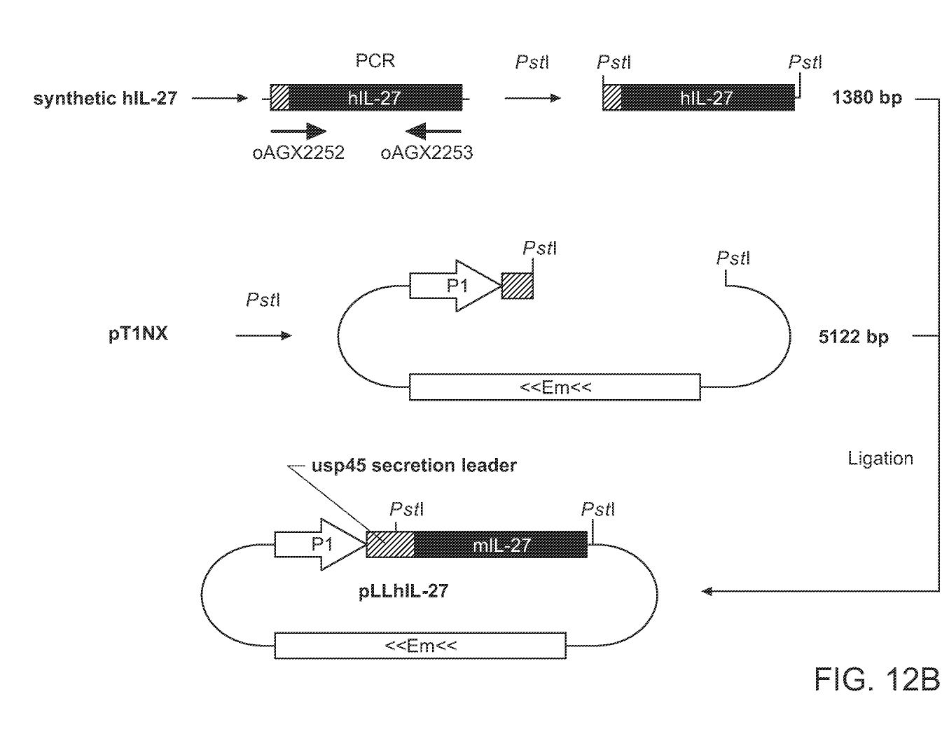

### Construction of Mouse IL-27 Nucleic Acid Molecules (FIG. 12A)

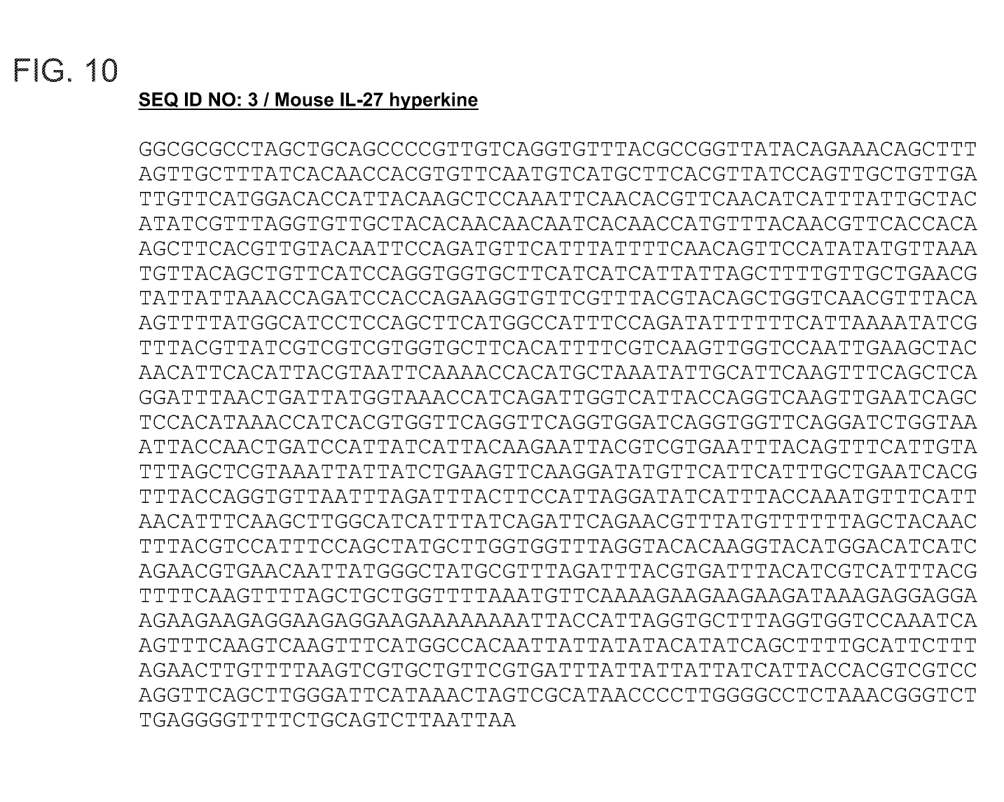

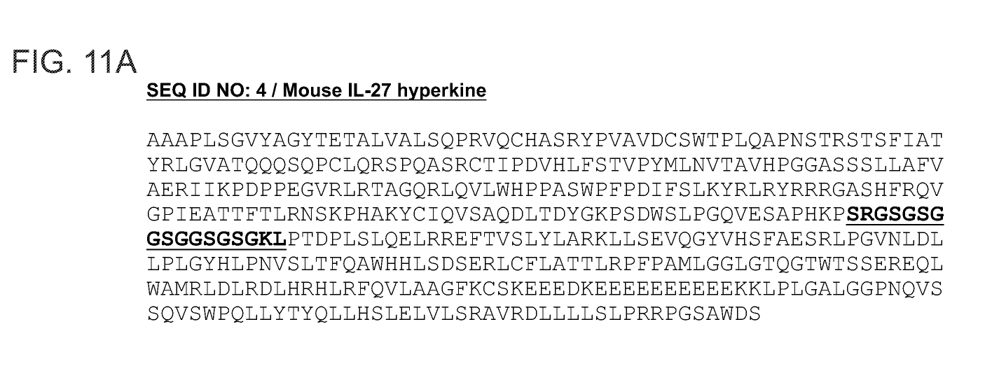

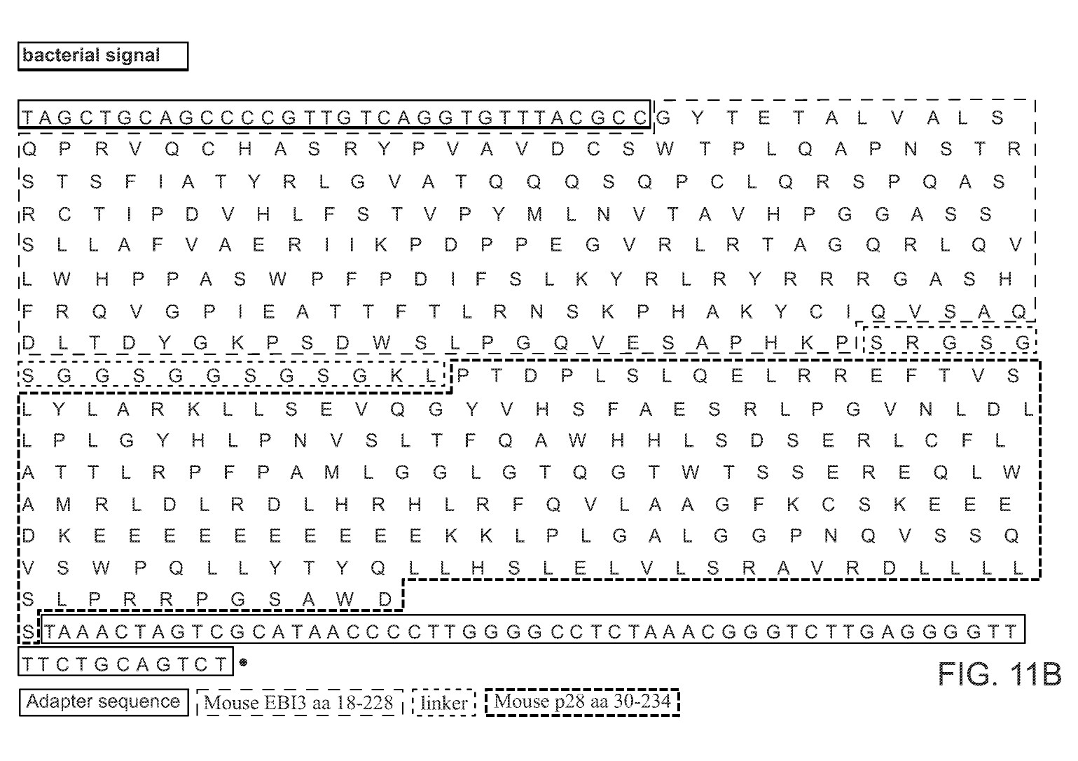

Synthetic mIL-27 hyperkine (1407 bp, SEQ ID NO: 3, FIG. 10) encoding mIL-27 alpha and beta chains joined by a peptide linker comprising the sequence N-SRGSGSGGSGGSGSGKL-C (SEQ ID NO: 5) was designed along preferential codon usage for L. lactis and avoidance of secondary structure, and was extended at the 5′ end with coding information for aa 18-27 of the secretion leader of L. lactis usp45 (GeneID: 4797218) and suitably positioned PstI restriction endonuclease sites at both the 5′ as well as the 3′ end (see FIG. 11). Gene synthesis was performed by GENEART, Inc. (Burlingame, Calif.). The synthetic mIL-27 gene was used as template DNA in a PCR reaction with oligonucleotides oAGX2252 (5′CCTAGCTGCAGCCCCG3′) (SEQ ID NO: 8) and oAGX2253 (5′AGACTGCAGAAAACCCCTC3′) (SEQ ID NO: 9). The 1394 by PCR fragment was purified and digested with PstI restriction enzyme to yield a 1380 by fragment. The cloning vector pT1NX was digested with PstI restriction enzyme to yield a 5122 by fragment. Ligation of both fragments resulted in pAGX0766 (ActoGeniX strain collection nr 1046). In this plasmid, mIL-27 is positioned downstream of the L. lactis P1 promoter (Waterfield et al., Gene 165:9-15 (1995)) and ligation of the 5′ PstI site results in fusion of mIL-27 to the secretion leader of L. lactis usp45 (gray). The DNA sequence of the expression fragment P1>>usp45 secretion leader>>mIL-27 was verified and showed 100% identity to that of the predicted.

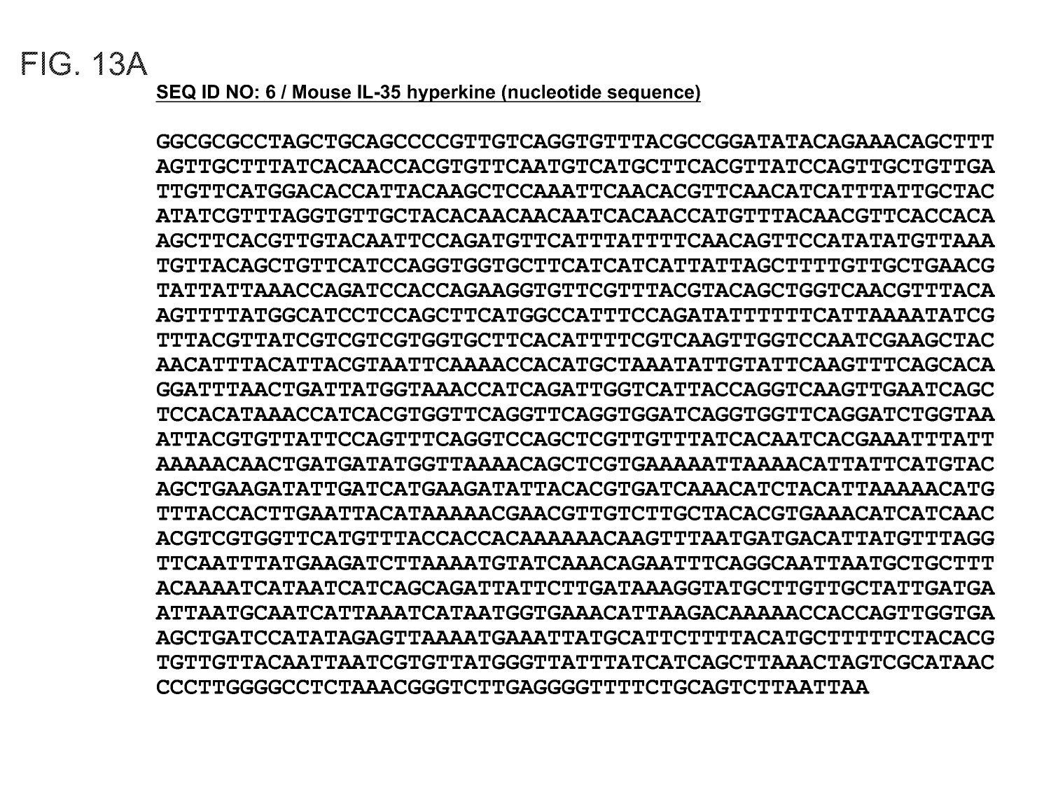

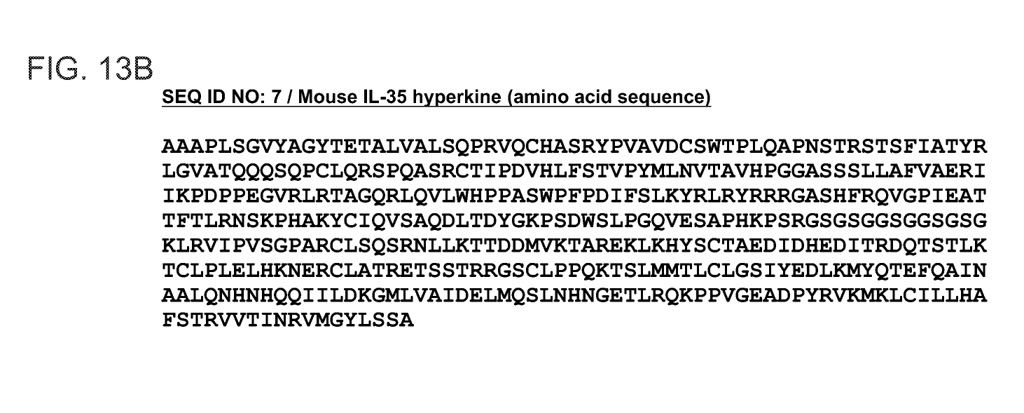

Synthetic mIL-35 hyperkine (FIGS. 13A and 13B) was similarly synthesized as described above.

### Example 2

### Construction of human IL-27 nucleic acid molecules (FIG. 12B)

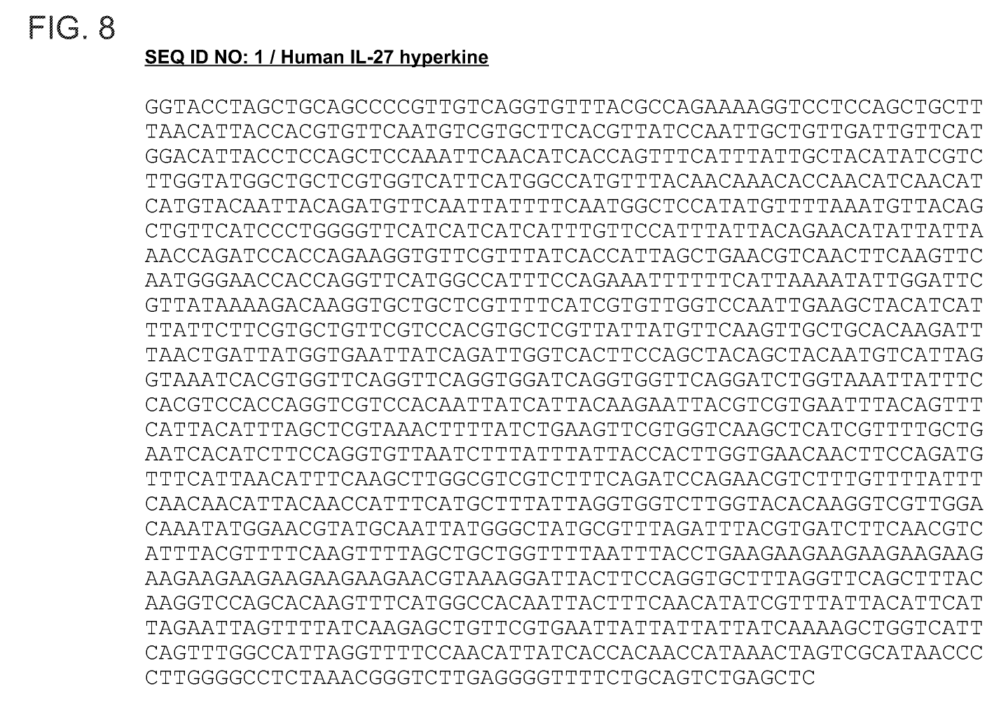

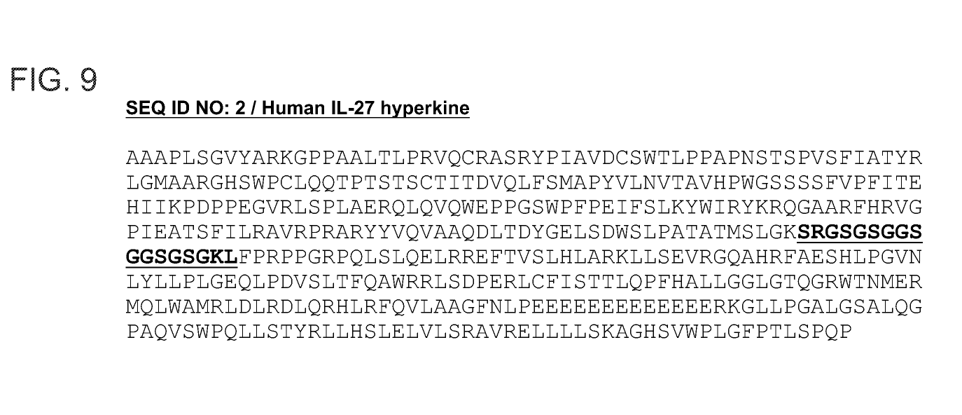

Synthetic hIL-27 is designed and subcloned following a similar strategy. Synthetic hIL-27 hyperkine (1428 bp, SEQ ID NO: 1; FIG. 8) encoding hIL-27 alpha and beta chains joined by a peptide linker comprising the sequence N-SRGSGSGGSGGSGSGKL-C (SEQ ID NO: 5) is designed based on preferential codon usage for L. lactis and avoidance of secondary structure, and was extended at the 5′ end with coding information for aa 18-27 of the secretion leader of L. lactis usp45 (GeneID: 4797218) and suitably positioned PstI restriction endonuclease sites at both the 5′ as well as the 3′ end (see FIG. 9). Gene synthesis is performed by GENEART, Inc. (Burlingame, Calif.). Synthetic hIL-27 gene is used as template DNA in a PCR reaction with oligonucleotides oAGX2252 (5′CCTAGCTGCAGCCCCG3′) (SEQ ID NO: 8) and oAGX2253 (5′AGACTGCAGAAAACCCCTC3′) (SEQ ID NO: 9). The 1418 bp PCR fragment is purified and digested with PstI restriction enzyme to yield a 1404 bp fragment. The cloning vector pT1NX is digested with PstI restriction enzyme to yield a 5122 bp fragment. Ligation of both fragments results in pLLhIL-27. In this plasmid, mIL-27 is positioned downstream of the L. lactis P1 promoter (Waterfield et al.) and ligation of the 5′ PstI site results in fusion of mIL-27 to the secretion leader of L. lactis usp45 (gray). The DNA sequence of the expression fragment P1>>usp45 secretion leader>>hIL-27 is verified and shows 100% identity to that of the predicted.

### Example 3

### L. lactis Expressing IL-27: A Therapeutic for Inflammatory Bowel Disease in a mouse model for IBD

Developing precise targeting of therapeutics to the intestinal mucosa is vital for the advancement of inflammatory bowel disease treatment (“IBD”). Thus, the objective of this Example is to demonstrate the development of a cost-effective, localized delivery of immunosuppresive cytokines that are actively synthesized in situ by a food-grade microcoorganism, e.g., Lactococcus lactis (L. lactis), to inhibit chronic IBD and prevent colon cancer development. L. lactis is a non-pathogenic, non-colonizing lactic acid bacterium that can be genetically engineered and orally formulated to deliver therapeutic proteins in the GI tract safely. As described below, oral administration of IL-27 or IL-35 secreting L. lactis results in local delivery of anti-inflammatory proteins to the colon, reducing inflammation in IBD mouse models and thus impeding colon cancer development. Not wishing to be bound by any theory, it is believed that as bacteria pass through the bowel, they secrete their recombinant proteins, which can then act locally in the bowel, without harmful passage to distant organs (e.g., lung).

This Example evidences that therapeutic application of genetically engineered L. lactis expressing IL-27 provides an effective and safe management of IBD, as well as cancer prevention, in humans. The data also demonstrate that IL-35 has no therapeutic benefit, contrary to published reports that indicate immunosuppressive effects of IL-35 (see, e.g., Bettini et al., Curr. Opin. Immun. 21:612-618 (2009)).

As noted above, IBD is a chronic inflammatory gastrointestinal disease that includes ulcerative colitis (“UC”) and Crohn's disease (“CD”). The incidence rate of IBD in western countries is about 1/1000. In addition, UC patients commonly develop colon cancer. The cause of IBD is thought to result from an aberrant attack of the immune system on the bowel. The bowel contains bacteria, and whereas normal individuals maintain just enough immunity to protect from these bacteria, IBD patients mount an overly strong immune response and damage their own tissues. Current treatment for IBD is systemic immunosuppression and/or surgical removal of the bowel or entire colon.

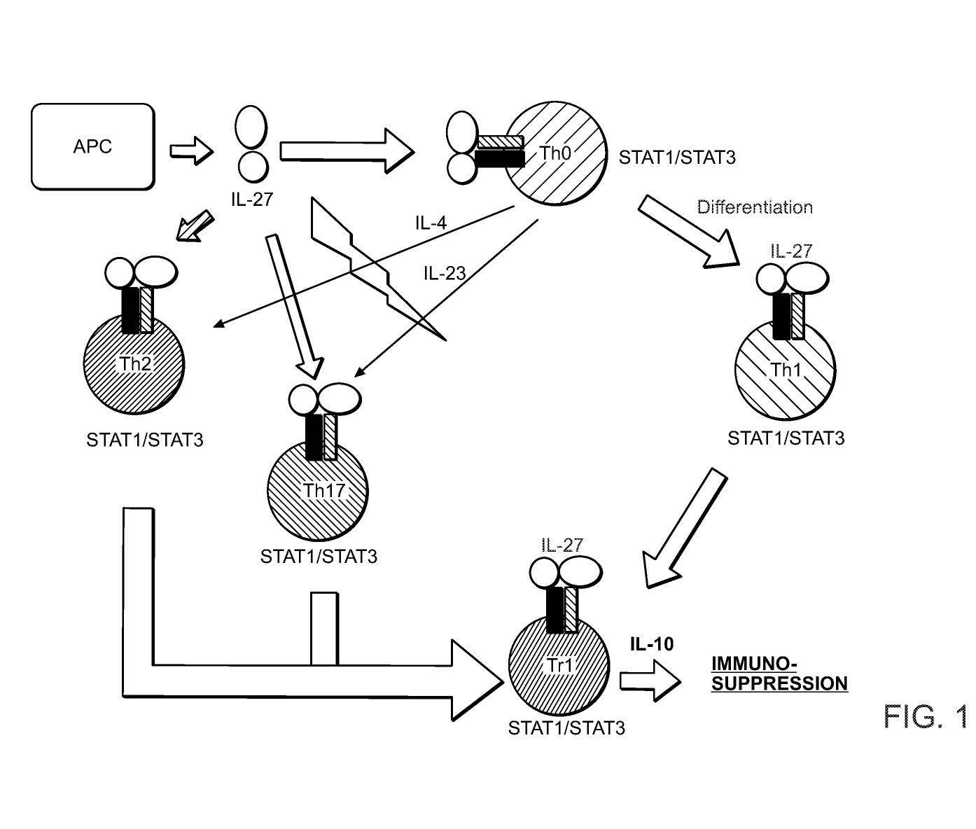

IL-27 and IL-35 are both heterodimeric cytokines that belong to the IL-12 cytokine family. Each is composed of an alph chain (IL-27: p28, IL-35: p35) and a beta chain (Ebi3). Mechanistically, IL-27 acts as an anti-inflammatory agent by suppressing Th17 cell development and promoting IL-10-producing T cell generation. See FIG. 1. Thus, it has both immunosuppressive, as well as, immunostimulative characteristics. IL-35 is produced by regulatory T cells (“Tregs”) and facilitate the Treg suppressive activity and promoting Treg generation.

In this Example, the food bacterium, L. lactis, has been engineered to express the immunosuppressive cytokine, IL-27. The recombinant bacterium was delivered to mice by oral gavage. These results show a striking therapeutic benefit of L. lactis-IL-27 in IBD induced by transfer of T cells. The results are as follows:

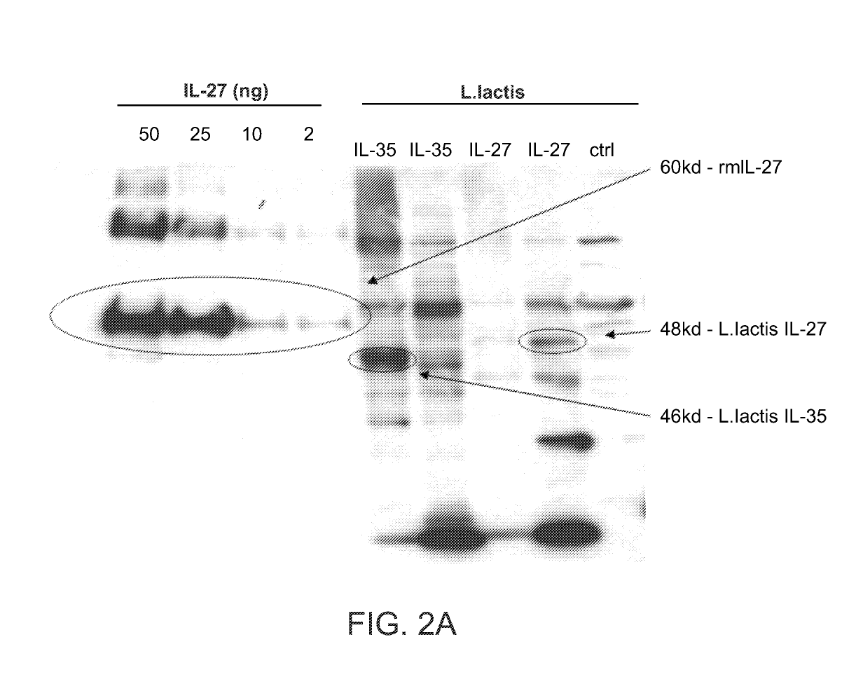

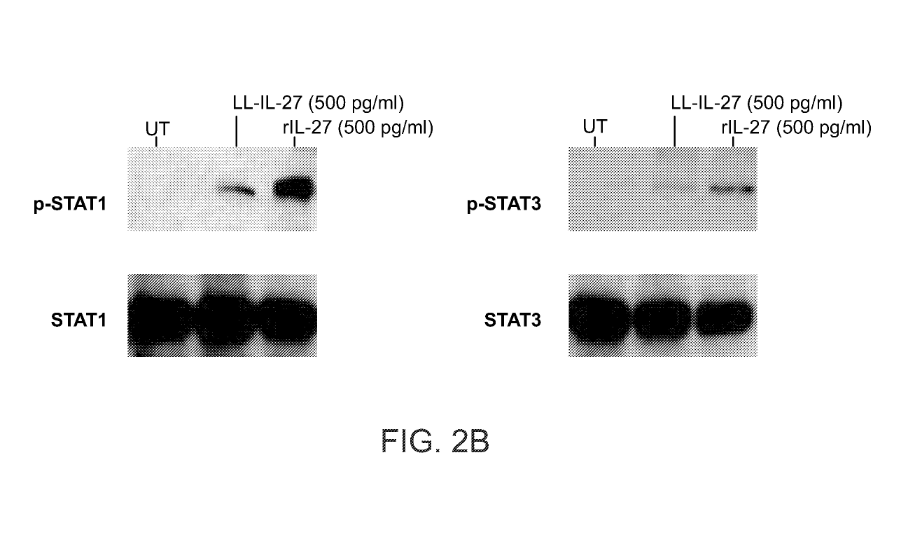

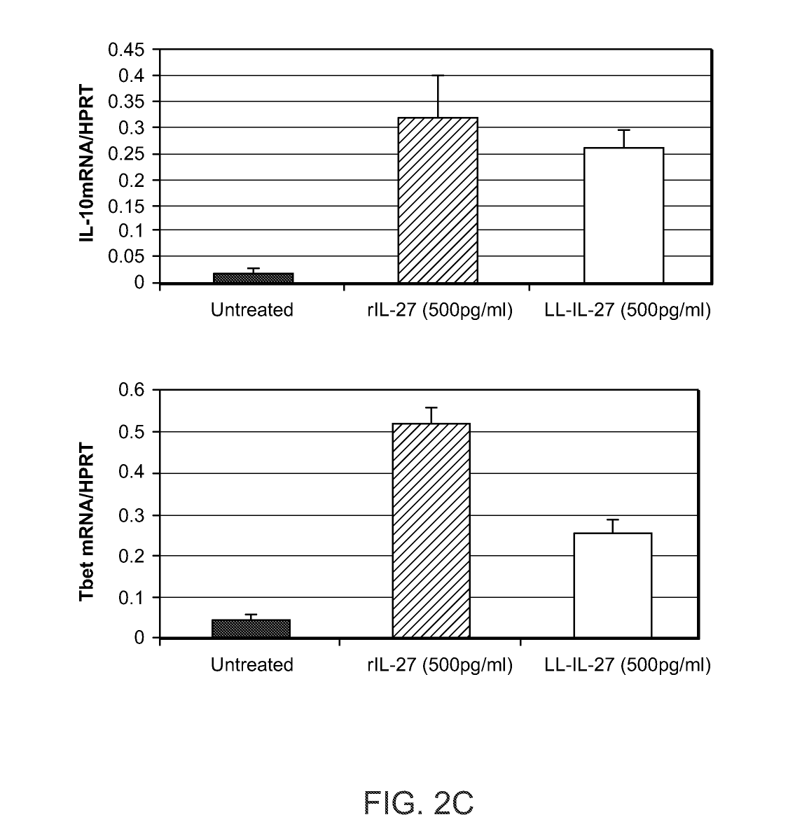

FIG. 2 shows the results of expression of IL-27 (mouse, SEQ ID NO: 2 (nucleotide sequence) and SEQ ID NO: 4 (amino acid sequence)) and IL-35 (mouse, SEQ ID NO: 6 (nucleotide sequence) and SEQ ID NO: 7 (amino acid sequence)) from genetically engineered L. lactis, as prepared in accordance with Example 1. FIG. 2A: Supernatants were collected from cultures of engineered L. lactis expressing either murine IL-27 or murine IL-35 and the proteins contained therein were separated by SDS-PAGE. Detectable anti-Ebi3 antibodies (“Ebi3” is the beta chain component of both IL-27 and IL-35) were used to detect IL-27 and IL-35 by Western blot. FIG. 2B: Biological activity of IL-27 was measured by detection of phosphorylation of Stat1 (“p-STAT1 detection”) and Stat3 (“p-STATS detection”) by Western blot. Stat1 and Stat3 are transcription factors that are important signaling molecule for many cytokines and growth-factor receptors. IL-27, when active, results in the phosphorylation of Stat1 and Stat3. FIG. 2C: Biological activity of IL-27 was also measured by increased IL-10 and Tbet production as determined by ELISA. Evaluation of a commercially available recombinant IL-27 (“rIL-27”) showed that there was somewhat less biological activity with L. lactis expressing recombinant IL-27 (“LL-IL-27”), which may be due to less efficient peptide folding or the presence of inhibitory factors in in vitro supernatants.

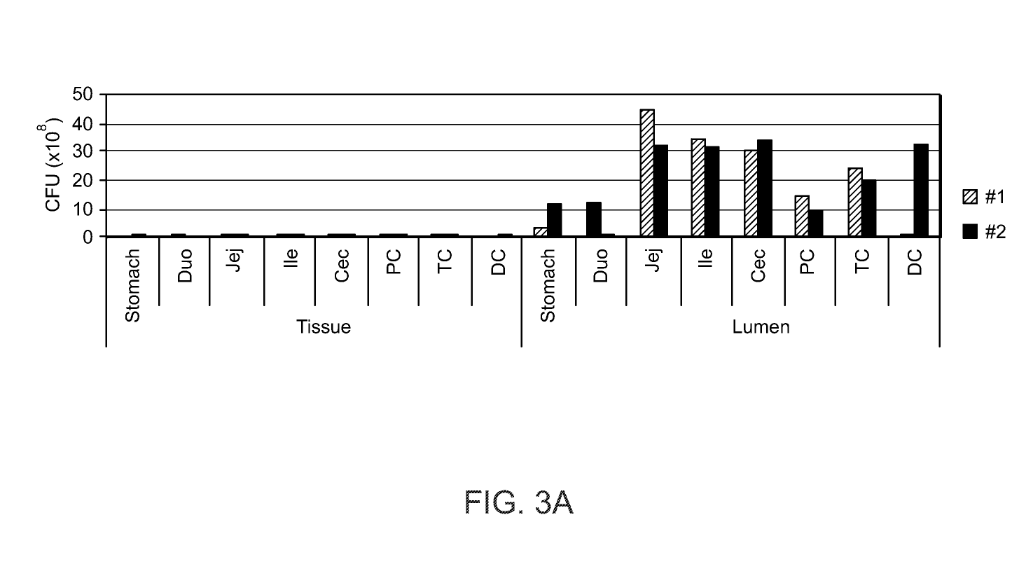

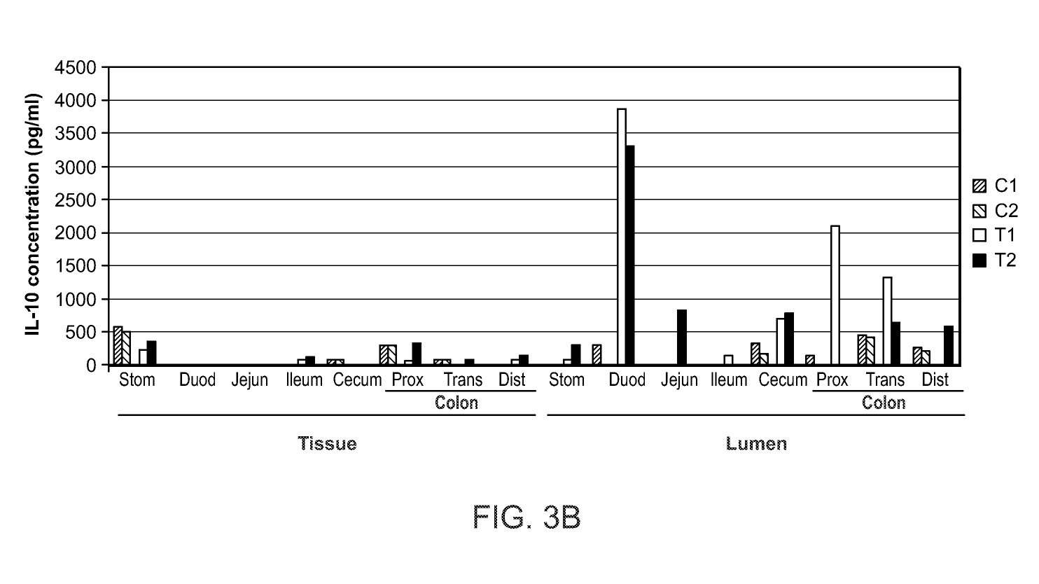

FIG. 3 shows that L. lactis harboring IL-27 survives in the digestive tract and is capable of local delivery of IL-27. LL-IL-27 was administered to normal C57Bl/6 male mice by oral gavage. Twelve hours later, different regions of the bowel were analyzed for surviving bacteria, detected by colonies resistant to erythromycin. Significant numbers of colony-forming units (CFU) were detected throughout the gut, as shown in two individuals (FIG. 3A). Living L. lactis were recovered from stomach, duodenum, jejunum, ileum, cecum, proximal, terminal and distal colon. Six hours after gavage, IL-10 was detected (FIG. 3B) in the luminal contents of various regions of LL-IL-27-treated mice (designated T) compared to LL-vector control (LL-vector)-treated mice (designated C). Thus, LL-IL-27 given by oral gavage was capable of acting locally in the target organ.

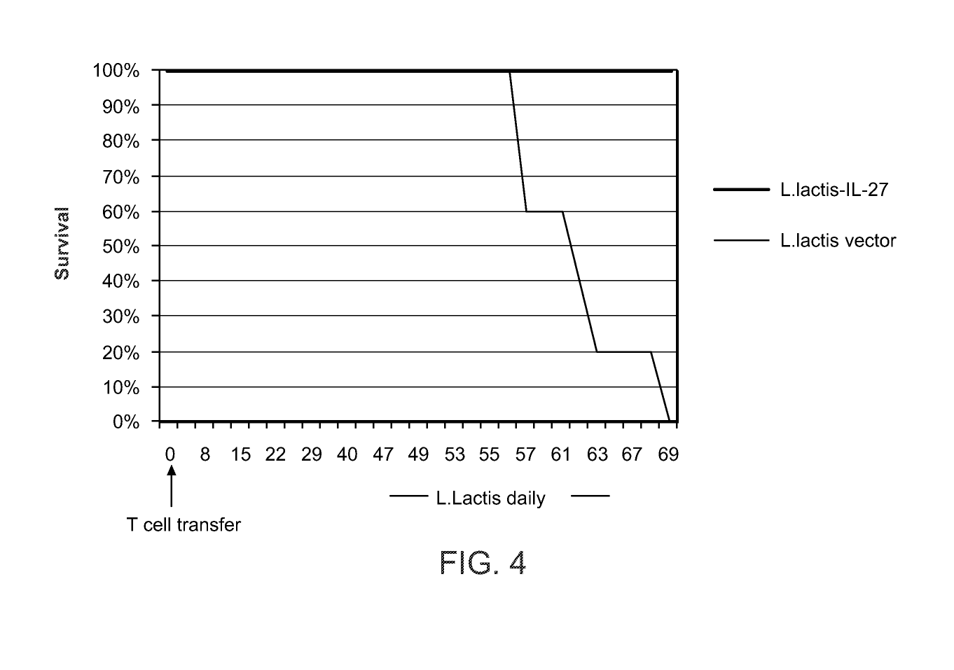

FIG. 4 shows the therapeutic effect of L. lactis-IL-27 on the T cell transfer mouse model of inflammatory bowel disease (discussed further herein). The T cell transfer model of IBD was used to evaluate any potential therapeutic benefit of LL-IL-27. Treatment was begun as symptoms developed, six weeks after transfer of CD45RB(hi) T cells in Rag1^{-/-} hosts. At day 69, mice treated with LL-IL-27 (n=5) were all healthy, while no mice treated with L. lactis-control vector (n=5) survived to the end point.

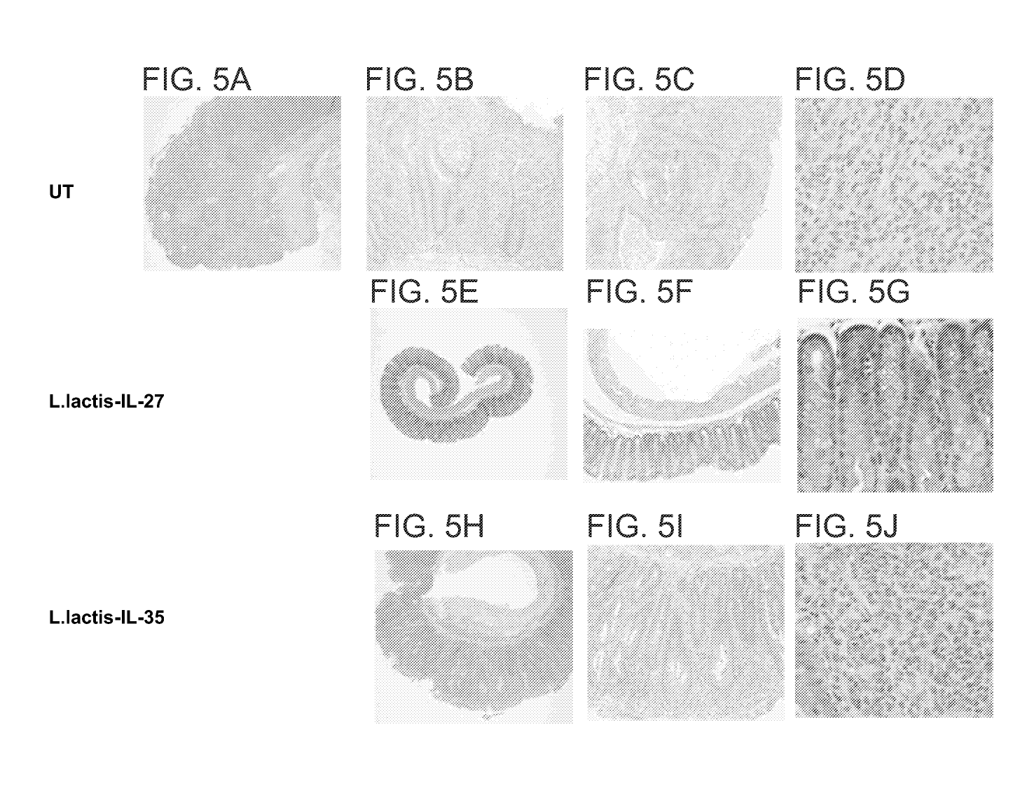

FIG. 5 provides histological evidence that L. lactis-IL-27 protects the distal colon from destruction of villi and inflammatory infiltration. No pathology was observed in the cells of the LL-IL-27 group except for a slight cellular infiltrate in one mouse, as compared to severe pathology in the LL-vector control group or another group that received L. lactis-IL-35 (LL-IL-35). Sections of distal colon (2 cm) were fixed in formalin, embedded in paraffin, and H&E staining was carried out according to routine procedure. FIGS. 5A-5D: Untreated mice. A) 4×, Colon mucosa greatly thickened with inflammatory infiltrate and hyperplastic crypts. B) 10×, Hyperplastic crypts depleted of goblet cells, crypt abscesses and inflammatory infiltrate in the mucosa. C) 10×, Gut intraepithelial neoplasia, crypt abscess and inflammatory infiltrate in the mucosa. D) 40×, Inflammatory infiltrate of mononuclear cells, neutrophils and eosinophils. FIGS. 5E-5G: L. lactis-IL-27 treated mice. E) 4×, Colon mucosa has normal histology. F) 10×, Normal colon crypts with goblet cells. G) 40×, Normal colon crypts with goblet cells, no inflammatory infiltrate. FIGS. 5H-5J: L. lactis-IL-35 treated mice. H) 10×, Colon mucosa thickened with inflammatory infiltrate and hyperplastic crypts. I) 10×, Hyperplastic crypts depleted of goblet cells and inflammatory infiltrate in the mucosa. J) 40×, Inflammatory infiltrate of mononuclear cells, neutrophils and eosinophils.

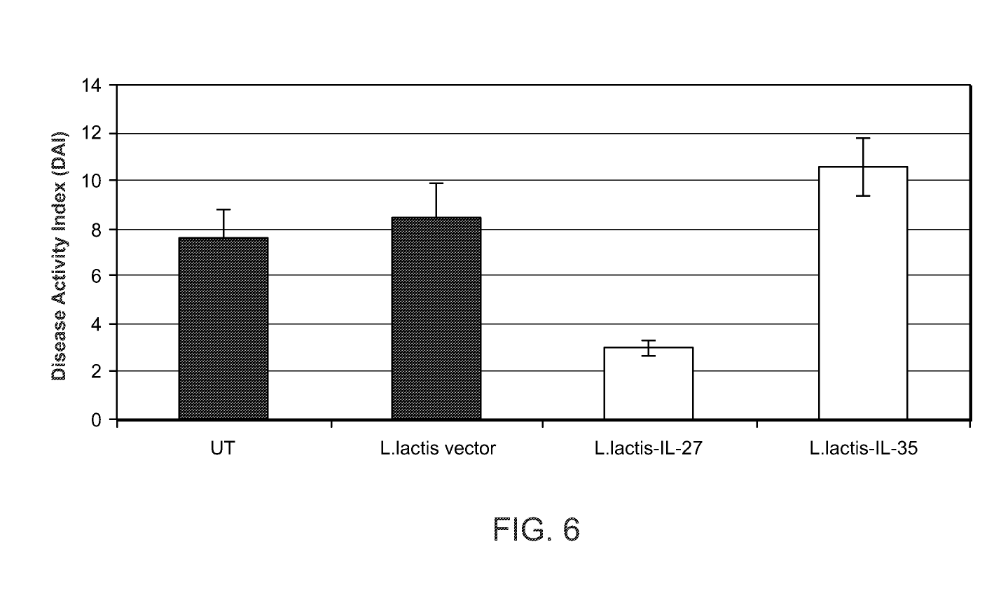

FIG. 6 depicts the protection afforded by LL-IL-27 versus untreated mice, LL-vector mice, and LL-IL-35 mice, as measured by several parameters of inflammatory bowel disease and reflected in the Disease Activity Index (DAI) (see Ostanin et al., Am. J. Physiol. Gastrointest. Liver Physiol. 296:G135-G146 (2009), which is incorporated herein by reference for more details regarding the T cell transfer model of chronic colitis). LL-IL-27 protected completely from appearance of occult blood in stool. In addition, mice treated with LL-IL-27 were associated with nearly normal stool consistency and partially relieved weight loss.

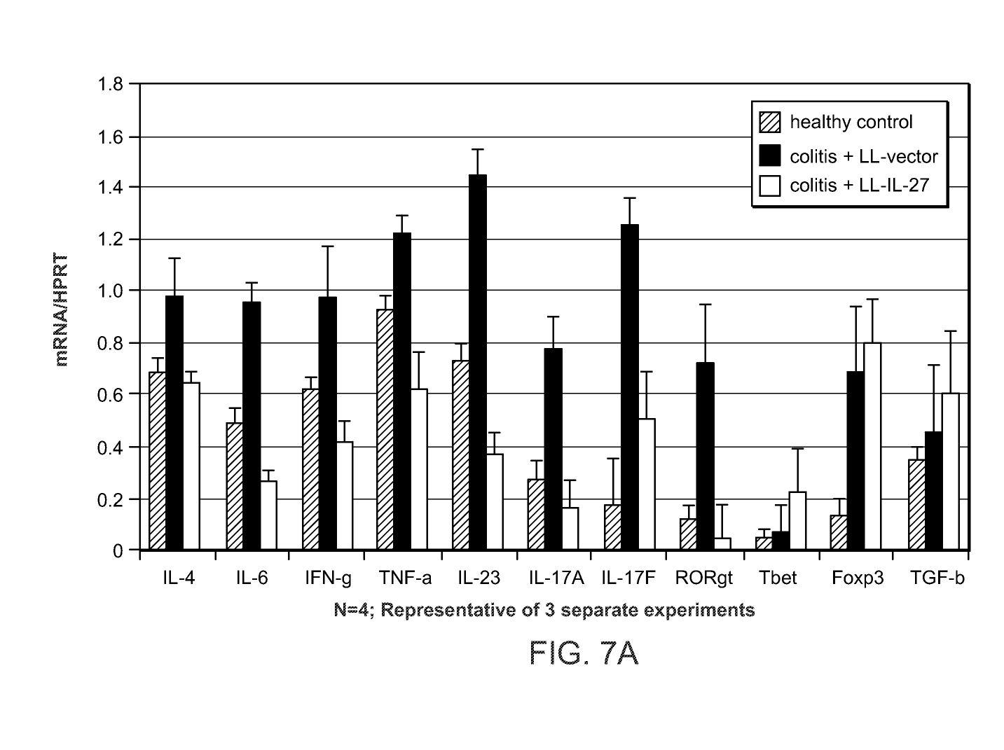

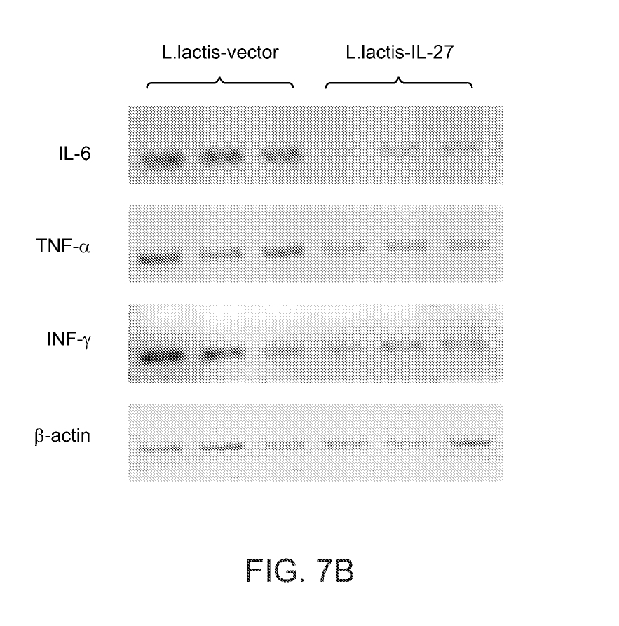

FIGS. 7A and 7B show the results of PCR analysis of the effects on the transcript levels of inflammatory cytokines in distal colons from three mice in each group tested above. There appears to be a reduction in IL-6, TNF-α and IFN-γ in the LL-IL-27 group as compared to the same reactions with the LL-vector control group.

### Example 4

### IL-10 is Required for the Therapeutic Effect of IL-27

Several cell types express the receptor complex for IL-27 and may be capable of responding, including T and B cells, NK cells, monocytes, mast cells, endothelial cells, Langerhans cells and dendritic cells. In this Example, it is demonstrated that T cells are a source of the detected bowel IL-10 induced by LL-IL-27.

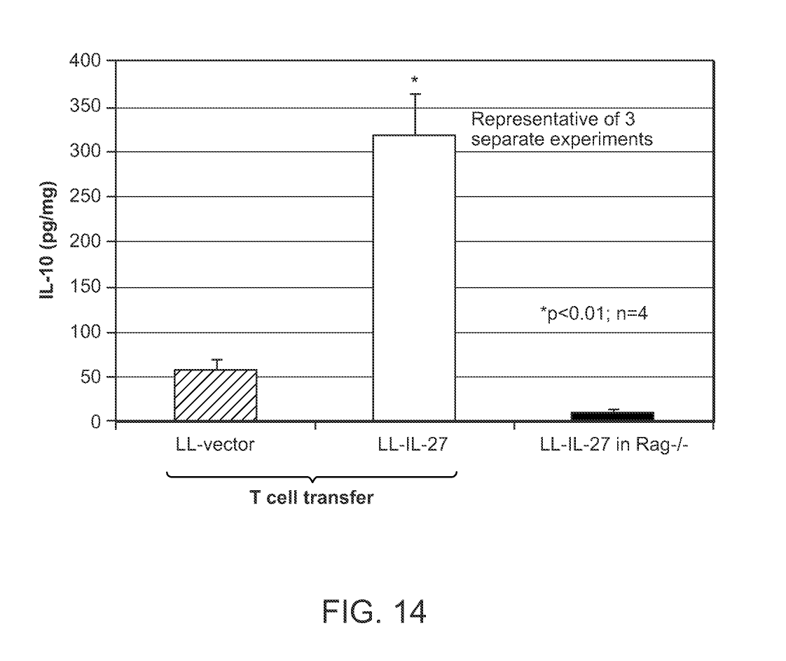

FIG. 14 shows that Rag^{-/-} mice treated LL-IL-27 do not induce expression of IL-10. These results indicate that induction of IL-10 requires the presence of T cells.

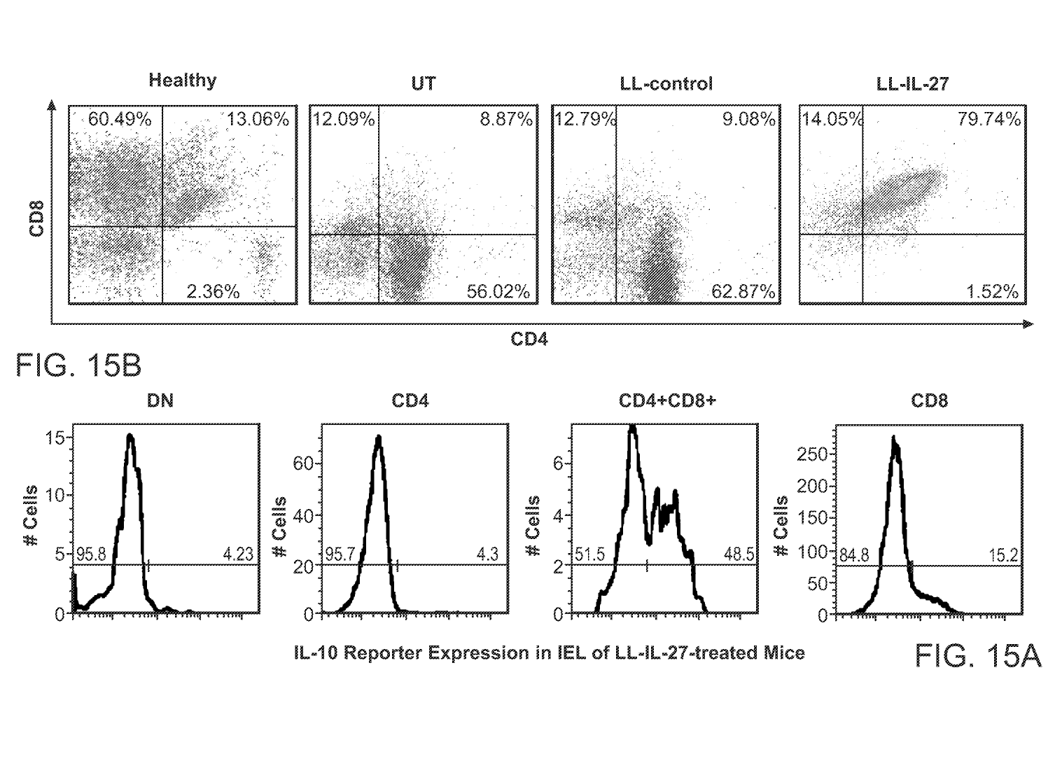

FIG. 15 identifies the IL-10 producing T cells in IBD mice treated with LL-IL-27. Intraepithelial cells (IEL) were isolated from healthy C57Bl/6 mice, untreated IBD Rag1^{-/-} mice (UT), IBD mice treated with LL-vector, and IBD mice treated with LL-IL-27. FIG. 15A: Analysis of the IEL cells identify the presence of a prominent CD4+CD8+ population in LL-IL-27 IBD mice. In contrast, healthy C57Bl/6 mice showed a predominance of CD8 cells; and IBD Rag1^{-/-} untreated mice and IBD mice treated with LL-vector showed CD4 infiltration. FIG. 15B: IBD was induced using induced using IL-10 reporter T cells, and the most prominent reporter expression observed in LL-IL-27 IBD mice was CD4+CD8+ cells.

### Example 5

### LL-IL-27 is more effective than LL-IL-10 in treating inflammatory bowel disease

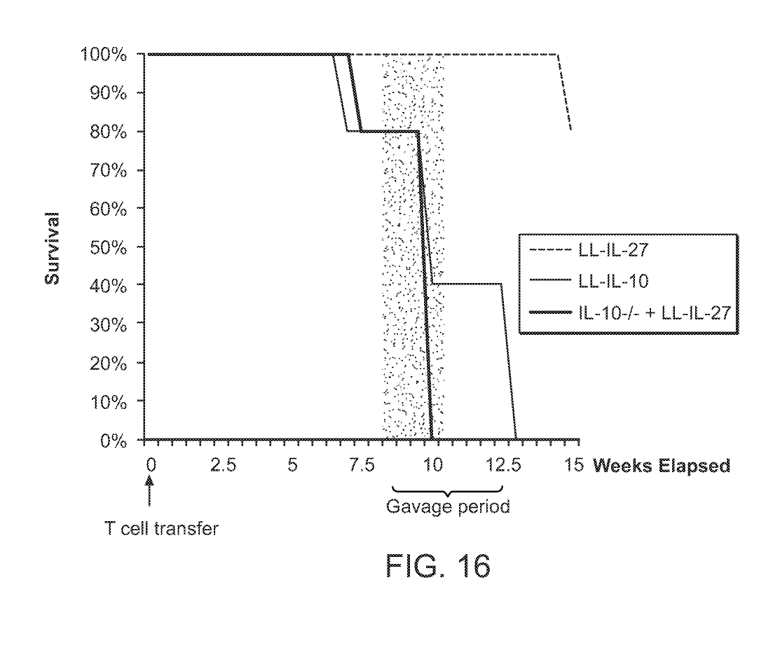

To further evaluate the relationship between IL-27 and IL-10, this Example compares the effects of L. lactis expressing IL-27 (LL-IL-27) and IL-10 (LL-IL-10) on IBD mice. As show in FIG. 16, none of the IBD mice treated with LL-IL-10 survived. At best, LL-IL-10 delayed the death of the treated mice. Surprisingly, the results showed that more LL-IL-27 mice survived, and those mice that died experienced a longer period of survival.

### Example 6

### E. faecium Expressing IL-27

This Example provides further evidence that microorganisms can be genetically engineered to express IL-27 for use to treat IBD, as well as cancer prevention, in humans.

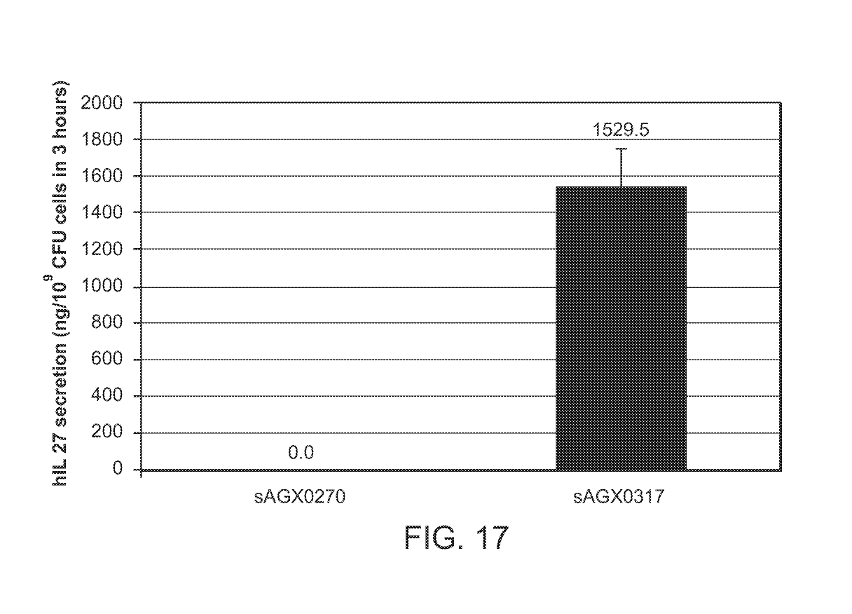

The DNA coding sequence of the usp45 secretion leader of Lactococcus lactis (SS) was fused in frame to the DNA sequence of mature human IL27. The construct was introduced into Enterococcus faecium and IL27 secretion was quantified by ELISA. As shown in FIG. 17, E. faecium is capable of secreting considerable amounts of heterologous human IL-27 protein.

### SUMMARY

At present, current treatments for IBD involve use of global immunosuppressants or surgery. Surgery is not a desirable treatment option as it is invasive, and use of global immunosuppressants are associated with adverse effects ranging from oral thrush and herpes zoster to more severe complications such as tuberculosis, histoplasmosis and coccidiomycosis. Immunosuppressive therapies also permit reactivation of endogenous viruses, which can cause increases in Epstein-Barr virus-induced lymphoma and JC virus-induced progressive multifocal leukoencephalophathy. In certain instances, it has been demonstrated that use of such therapies results in increased non-melanoma skin cancer and an unusual hepatosplenic T cell lymphoma. Accordingly, there is a need to find additional methods of treatment.

As reported herein, delivery of IL-27 is a novel treatment strategy for IBD that does not suffer from the above-described problems. The results confirm by in vitro studies that genetically engineered L. lactis expressing IL-27 or IL-35 protein and that L. lactis-IL-27 is bioactive. The results further determined the concentration of the IL-27/p28 produced by the L. lactis-IL-27.

The in vivo studies described herein demonstrate the surprising and dramatic effect of LL-IL-27 as a therapeutic for treating IBD. Local administration of IL-27 resulted in 100% survival of the test mice, low DAI, and normal histology, while the other treatment groups had low to no survival, high DAI, severe colon inflammation and crypt abscesses. These results are surprising and unexpected because even though the effects of IL-27 are mediated through increased expression of IL-10 production, the inventors have discovered that local delivery of IL-27 is dramatically more effective than treatment with IL-10. In addition, these results are even more unpredictable in view of the fact that IL-27 is known as both a pro- and anti-inflammatory cytokine.

The results reported herein were obtained using the following methods and materials.

### Mouse IL-27/-35 Hyperkines

The mouse IL-27 and IL-35 hyperkines were designed by incorporating a linker sequence (SRGSGSGGSGGSGSGKL) between the EBI3 and p28 (IL-27) or p35 (IL-35) sequences. DNA sequences with optimal L. lactis codon usage were synthesized by Geneart (Burlingame, Calif.). The Usp45 secretion signal, which encodes a secreted protein from L. lactis strain MG1363 was fused to the hyperkines downstream of the lactococcal P1 promoter.

### Bacteria

L. lactis strain MG1363 and E. faecium strains sAGX0270 and sAGX0317 were used throughout this study. Bacteria were cultured in GM17E medium, i.e., Difco M17 broth (BD, Franklin Lakes, N.J.) supplemented with 0.5% glucose (Sigma, St. Louis, Mo.). For L. lactis, the broth was further supplemented with 5 μg/ml erythromycin (Sigma). Stock suspensions were stored at −80° C. in 50% glycerol (Sigma) in GM17E. For intragastric inoculations, stock suspensions were diluted 1000-fold in fresh GM17E and incubated for 16 h at 30° C., reaching a saturation density of 2×10⁹ CFU per ml. Bacteria were harvested by centrifugation and concentrated 10-fold in BM9 medium. Treatment doses consisted of 100 μl of this suspension.

### Protein Expression and Immunoblotting

L. lactis strains were routinely grown as standing cultures at 30° C. For the analysis of protein expression and secretion, saturated cultures, grown in GM17E, were diluted 1/100 and grown for 3 h in fresh buffered M9 salt medium. Bacteria and culture supernatants were separated by centrifugation at 1500 g during 10 min. Supernatants were run on a 4-12% Bis-Tris gel under reducing conditions. Ebi3 expression was detected using anti-Ebi3 (Santa Cruz Biotechnology, Inc, Santa Cruz, Calif.) as a primary antibody in standard western blotting procedures. Recombinant mouse IL-27 (rmIL-27) (R & D Systems, Inc., Minneapolis, Minn.) was used as a positive control. IL-27 concentration in L. lactis supernatants was determined by ELISA using Quantikine Mouse IL-27 p28 (R &D Systems) (LOD: 1.5 pg/ml).

E. faecium strains were inoculated from single colony into 10 ml GM17 supplemented with 200 μM thymidine (GM17T) and grown for 16 hours at 30° C. For the quantification of hIL27 secretion, these saturated overnight cultures were diluted 1/25 in 5 ml fresh GM17T medium and grown for 4 hours at 30° C. Cells were collected by centrifugation at 3220×g for 10 minutes, resuspended in an equal amount BM9T medium and cultured for another 3 hours at 30° C. BM9T contains M9 salts, 0.5% casitone, 0.5% glucose, 25 mM NaHCO₃, 25 mM Na₂CO₃, 2 mM MgSO₄, 0.1 mM CaCl₂ and 200 μM thymidine. Cells and culture supernatants were separated by centrifugation at 3220×g for 10 minutes. The amount of secreted human hIL27 in the culture supernatant was quantified by sandwich hIL27 ELISA (R&D systems). All strains were treated in parallel.

Bioactivity of L. lactis-IL-27

The bioactivity of L. lactis-IL-27 strains was analyzed using phosphorylation of STAT-1/3 and induction of IL-10 and T-bet as read outs. For each bioactivity assay, splenic naïve CD4⁺ T cells were isolated from C57Bl/6 mice. For the p-STAT-1/3 assay, cells were stimulated with anti-CD3/28 (eBioscience, San Diego, Calif.) and rmIL-27 (500 pg/ml) (R&D, Minneapolis, Minn.), L. lactis control vector, or L. lactis-IL-27 (500 pg/ml) strains for 20 min at 37° C. Prepared lysates were run on 4-12% Bis-Tris gel. p-STAT-1/3 expression was detected using phospho-STAT1 (Tyr701) and phospho-STAT3 (Tyr705) (Cell Signaling Technology, Danvers, Mass.) as primary antibodies and STAT1 and STAT3 (124H6) (Cell Signaling Technology) antibodies as loading controls, in standard western blotting procedures. For the IL-10 protein induction assay, cells were stimulated with anti-CD3/28 and rmIL-27 (390 pg/ml), L. lactis control vector, or L. lactis-IL-27 (390 pg/ml) strains for 72 hours. Supernatants were analyzed using READY-SET-GO! Mouse IL-10 ELISA (eBioscience) (LOD: 30 pg/ml). For analysis of IL-10 and Tbet mRNA induction, cells were stimulated with anti-CD3/28 and rmIL-27 (500 pg/ml), L. lactis control vector, or L. lactis-IL-27 (500 pg/ml) strains for 2 hours. Total RNA was extracted from cells with Qiagen RNeasy Mini Kit (Valencia, Calif.) according to manufacturer's protocol. Reverse transcription was performed using SuperScript III First-Strand Synthesis System (Invitrogen; Carlsbad, Calif.) according to manufacturer's protocol. PCR amplification was achieved using Platinum PCR Supermix (Invitrogen) and oligonucleotide primers (Integrated DNA Technologies; Coralville, Iowa). The amplified products were separated by electrophoresis on a 1.5% agarose gel and visualized using SYBR Safe DNA gel stain (Invitrogen). To semi-quantify the induction of mRNA expression, transcript levels were normalized relative to the expression of HPRT mRNA using densitometric analysis by ImageJ 1.41 software.

In vivo delivery of IL-27 after L. lactis administration

LL-IL-27 was administered to normal C57Bl/6 male mice by oral gavage. Twelve hours later, different regions of the bowel were analyzed for surviving bacteria, detected by colonies resistant to erythromycin. In addition, IL-10 was detected in the luminal contents of various regions of the LL-IL-27-treated mice.

### Induction of Colitis by T Cell Transfer, L. Lactis Administration

Immunodeficient Rag^{-/-} females on C57Bl/6 background were used for recipients, while an equal number of male C57Bl/6 mice were used for donors. Single cell suspensions were made from harvested spleens. CD4+ T cells were enriched using MACS CD4+ T cell Isolation Kit (Miltenyi Biotec Inc., Auburn, Calif.). CD4+ T cells were fluorescently labeled using anti-CD4-APC and anti-CD45RB-FITC (BD Pharmingen, Franklin Lakes, N.J.). CD4+ CD45RBhigh cells were sorted by flow cytometry and injected in the recipient mice. Colitis was induced approximately 6 weeks following cell transfer. Treatment groups included: untreated, L. lactis control vector, L. lactis-IL-27, L. lactis-IL-35, and L. lactis-IL-10. L. lactis administration began following colitis induction and continued with 14 daily gavages. Mice were either harvested at death or on day 69 of the experiment (or as indicated in the figures).

### Disease Activity Index (DAI)

Following cell transfer, mice were monitored twice a week prior to L. lactis administration and then daily once L. lactis administration began. Monitoring included analysis of body weight, stool consistency, and occult in stool. A score for each parameter was given based on the scale below. DAI represents the combined parameter scores.

### Histological Analysis

The colon was removed, cleaned, and opened longitudinally. Two cm sections from the distal and proximal colon were fixed in formalin solution and embedded in paraffin. Sections were stained with hematoxylin and eosin (H&E) and analyzed by the Laboratory Animal Services Program, SAIC.

Having thus described in detail embodiments of the present invention, it is to be understood that the invention defined by the above paragraphs is not to be limited to particular details set forth in the above description as many apparent variations thereof are possible without departing from the spirit or scope of the present invention.

1. A method of treating inflammatory bowel disease, mucosal inflammatory pathology or intestinal inflammatory pathology in a subject in need thereof, said method comprising: locally administering to the intestinal mucosa of the subject a therapeutically effective amount of IL-27 or a therapeutic variant or fragment thereof.

2. The method of claim 1, wherein the IL-27 is administered using a gastrointestinal delivery system.

3. The method of claim 2, wherein the gastrointestinal delivery system is a recombinant microorganism effective to produce the IL-27 in situ in the intestinal mucosa in the subject.

4. The method of claim 3, wherein the recombinant microorganism is a microflora species.

5. The method of claim 4, wherein the microflora species belongs to the bacterial genera of Bacteriodes, Clostridium, Fusobacterium, Eubacterium, Ruminococcus, Peptococcus, Eschericia, Lactobacillus, Enterococcus, or Lactococcus.

6. The method of claim 3, wherein the recombinant microorganism is a non-commensal and non-colonizing bacterial species.

7. The method of claim 6, wherein the recombinant microorganism is a gram positive bacteria.

8. The method of claim 7, wherein the bacterial species belongs to the Lactococcus or Enterococcus genera.

9. The method of claim 2, wherein the gastrointestinal delivery system is a microparticle comprising IL-27 or therapeutic variant or fragment thereof.

10. The method of claim 9, wherein the microparticle further comprises a coating that enables controlled release of the IL-27 or therapeutic variant or fragment thereof into the gastrointestinal tract.

11. The method of claim 10, wherein the coating further enables continuous or sustained release of the IL-27 or therapeutic variant or fragment thereof.

12. The method of claim 1, wherein the inflammatory bowel disease is Crohn's Disease.

13. The method of claim 1, wherein the inflammatory bowel disease is ulcerative colitis.

14. The method of claim 1, wherein the therapeutically effective amount of the IL-27 is sufficient to reduce intestinal mucosal inflammation by at least 10-90%.

15. (canceled)

16. The method of claim 1, wherein the method further comprises co-administering a second therapeutic agent.

17. The method of claim 16, wherein the second therapeutic agent is a corticosteroid, sulphasalazine, derivative of sulphasalazine, immunosuppressive drug, cyclosporin A, mercaptopurine, azathioprine, cytokine, or cytokine antagonist.

18-19. (canceled)

20. A method of treating a condition sensitive to IL-27 in a subject in need thereof, said method comprising: administering to the subject a recombinant microorganism capable of producing a therapeutically effective amount of IL-27 or a therapeutic variant or fragment thereof.

21. The method of claim 20, wherein the recombinant microorganism produces the IL-27 in situ in the intestinal mucosa of the subject.

22. The method of claim 20, wherein the recombinant microorganism is a microflora species.

23-35. (canceled)

36. A pharmaceutical composition comprising a recombinant microorganism capable of producing a therapeutically effective amount of IL-27 in situ in a tissue of the gastrointestinal tract.

37-41. (canceled)

42. A pharmaceutical composition comprising recombinant Lactococcus lactis that is capable of expressing a therapeutically effective amount of IL-27 in situ in a tissue of the gastrointestinal tract.

43. A microparticle comprising IL-27 suitable for release of the active ingredient in the gastrointestinal tract, wherein the microparticle comprises a coating that enables controlled release of the IL-27 or therapeutic variant or fragment thereof into the gastrointestinal tract.

44. A pharmaceutical composition comprising the microparticle of claim 43.

45. A kit comprising a recombinant microorganism capable of producing a therapeutically effective amount of IL-27 in situ in the intestinal mucosa and instructions for use in treating inflammatory bowel disease.

46. The kit of claim 45, wherein the recombinant microorganism is a microflora species.

47-50. (canceled)

51. A kit comprising a microparticle suitable for release of the active ingredient in the gastrointestinal tract, wherein the microparticle comprises IL-27 and a coating that enables controlled release of the IL-27 or therapeutic variant or fragment thereof into the gastrointestinal tract.

52-57. (canceled)

58. The method of claim 1, wherein the subject is a human.

59. The method of claim 1, wherein the IL-27 is encoded by the nucleotide sequence of SEQ ID NO: 1 (human) or SEQ ID NO: 3 (mouse).

60. The method of claim 1, wherein the IL-27 has the amino acid sequence of SEQ ID NO: 2 (human) or SEQ ID NO: 4 (mouse).

61. The pharmaceutical composition of claim 36, wherein the IL-27 is encoded by the nucleotide sequence of SEQ ID NO: 1 (human) or SEQ ID NO: 3 (mouse).

62. The pharmaceutical composition of claim 36, wherein the IL-27 has the amino acid sequence of SEQ ID NO: 2 (human) or SEQ ID NO: 4 (mouse).
